SEQUENCE LISTING (33 sequences)

SEQ ID NO: 1 - DNA, 1365 bp, Homo sapiens
  misc_feature: TNFRSF1A
atgggcctctccaccgtgcctgacctgctgctgccgctggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccccagattgagaatgttaagggcactgaggactcaggcaccacagtgctgttgcccctggtcattttctttggtctttgccttttatccctcctcttcattggtttaatgtatcgctaccaacggtggaagtccaagctctactccattgtttgtgggaaatcgacacctgaaaaagagggggagcttgaaggaactactactaagcccctggccccaaacccaagcttcagtcccactccaggcttcacccccaccctgggcttcagtcccgtgcccagttccaccttcacctccagctccacctatacccccggtgactgtcccaactttgcggctccccgcagagaggtggcaccaccctatcagggggctgaccccatccttgcgacagccctcgcctccgaccccatccccaacccccttcagaagtgggaggacagcgcccacaagccacagagcctagacactgatgaccccgcgacgctgtacgccgtggtggagaacgtgcccccgttgcgctggaaggaattcgtgcggcgcctagggctgagcgaccacgagatcgatcggctggagctgcagaacgggcgctgcctgcgcgaggcgcaatacagcatgctggcgacctggaggcggcgcacgccgcggcgcgaggccacgctggagctgctgggacgcgtgctccgcgacatggacctgctgggctgcctggaggacatcgaggaggcgctttgcggccccgccgccctcccgcccgcgcccagtcttctcaga

SEQ ID NO: 2 - protein, 455 aa, Homo sapiens
  MISC_FEATURE: Wild Type TNF Receptor 1
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 3 - DNA, 591 bp, Homo sapiens
  misc_feature: Ligand Binding Domain of TNFR1
atgggcctctccaccgtgcctgacctgctgctgccgctggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctacca

SEQ ID NO: 4 - protein, 197 aa, Homo sapiens
  MISC_FEATURE: Ligand Binding Domain of TNFR1
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLP

SEQ ID NO: 5 - DNA, 1008 bp, Homo sapiens
  misc_feature: full-length FAS
atgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgaagagaaaggaagtacagaaaacatgcagaaagcacagaaaggaaaaccaaggttctcatgaatctccaactttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctag

SEQ ID NO: 6 - protein, 335 aa, Homo sapiens
  MISC_FEATURE: full-length FAS
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 7 - DNA, 505 bp, Homo sapiens
  misc_feature: Effector Domain of FAS
aggatccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctag

SEQ ID NO: 8 - protein, 167 aa, Homo sapiens
  MISC_FEATURE: Effector Domain of FAS
GSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 9 - DNA, 1101 bp, Homo sapiens
  misc_feature: FAS-chimera
atgggcctctccaccgtgcctgacctgctgctgccgctggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccaagcttaggatccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctag

SEQ ID NO: 10 - protein, 366 aa, Homo sapiens
  MISC_FEATURE: FAS-chimera
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPSLGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 11 - DNA, 47 bp, Artificial Sequence
  Endothelial Cell-Specific Enhancer Elements
ctggagggtgactttgcttctggagccagtacttcatacttttcatt

SEQ ID NO: 12 - DNA, 47 bp, Artificial Sequence
  Endothelial Cell-Specific Enhancer
aatgaaaagtatgaagtactggctccagaagcaaagtcaccctccag

SEQ ID NO: 13 - DNA, 44 bp, Artificial Sequence
  Endothelial Cell-Specific Enhancer
gtacttcatacttttcattccaatggggtgactttgcttctgga

SEQ ID NO: 14 - DNA, 44 bp, Artificial Sequence
  Endothelial Cell-Specific Enhancer Element
tccagaagcaaagtcaccccattggaatgaaaagtatgaagtac

SEQ ID NO: 15 - DNA, 131 bp, Artificial Sequence
  Endothelial Cell-Specific 3X Enhancer Element
ctccagaagcaaagtcaccccattggaatgaaaagtatgaagtacaatgaaaagtatgaagtactggctccagaagcaaagtcaccctccagaagcaaagtcaccccattggaatgaaaagtatgaagtac

SEQ ID NO: 16 - DNA, 131 bp, Artificial Sequence
  Endothelial Cell-Specific 3X Enhancer Element
gtacttcatacttttcattccaatggggtgactttgcttctggagggtgactttgcttctggagccagtacttcatacttttcattgtacttcatacttttcattccaatggggtgactttgcttctggag

SEQ ID NO: 17 - DNA, 850 bp, Artificial Sequence
  Endothelial Cell-Specific PPE-1 Promoter
gtacgtgtacttctgatcggcgatactagggagataaggatgtgcctgacaaaaccacattgttgttgttatcattattatttagttttccttccttgctaactcctgacggaatctttctcacctcaaatgcgaagtactttagtttagaaaagacttggtggaaggggtggtggtggaaaagtagggtgatcttccaaactaatctggttccccgcccgccccagtagctgggattcaagagcgaagagtggggatcgtccccttgtttgatcagaaagacataaaaggaaaatcaagtgaacaatgatcagccccacctccaccccacccccctgcgcgcgcacaatacaatctatttaattgtacttcatacttttcattccaatggggtgactttgcttctggagaaactcttgattcttgaactctggggctggcagctagcaaaaggggaagcgggctgctgctctctgcaggttctgcagcggtctctgtctagtgggtgttttctttttcttagccctgcccctggattgtcagacggcgggcgtctgcctctgaagttagccgtgatttcctctagagccgggtcttatctctggctgcacgttgcctgtgggtgactaatcacacaataacattgtttagggctggaatgaagtcagagctgtttacccccactctataggggttcaatataaaaaggcggcggagaactgtccgagtcagaagcgttcctgcaccggcgctgagagcctgacccggtctgctccgctgtccttgcgcgctgcctcccggctgcccgcgacgctttcgccccagtggaagggccacttgctgcggccgc

SEQ ID NO: 18 - DNA, 987 bp, Artificial Sequence
  Endothelial Cell-Specific PPE-1 3X Promoter
gtacgtgtacttctgatcggcgatactagggagataaggatgtgcctgacaaaaccacattgttgttgttatcattattatttagttttccttccttgctaactcctgacggaatctttctcacctcaaatgcgaagtactttagtttagaaaagacttggtggaaggggtggtggtggaaaagtagggtgatcttccaaactaatctggttccccgcccgccccagtagctgggattcaagagcgaagagtggggatcgtccccttgtttgatcagaaagacataaaaggaaaatcaagtgaacaatgatcagccccacctccaccccacccccctgcgcgcgcacaatacaatctatttaattgtacttcatacttttcattccaatggggtgactttgcttctggagaaactcttgattcttgaactctggggctggcagctagcctccagaagcaaagtcaccccattggaatgaaaagtatgaagtacaatgaaaagtatgaagtactggctccagaagcaaagtcaccctccagaagcaaagtcaccccattggaatgaaaagtatgaagtacgctagcaaaaggggaagcgggctgctgctctctgcaggttctgcagcggtctctgtctagtgggtgttttctttttcttagccctgcccctggattgtcagacggcgggcgtctgcctctgaagttagccgtgatttcctctagagccgggtcttatctctggctgcacgttgcctgtgggtgactaatcacacaataacattgtttagggctggaatgaagtcagagctgtttacccccactctataggggttcaatataaaaaggcggcggagaactgtccgagtcagaagcgttcctgcaccggcgctgagagcctgacccggtctgctccgctgtccttgcgcgctgcctcccggctgcccgcgacgctttcgccccagtggaagggccacttgctgcggccgc

SEQ ID NO: 19 - DNA, 35207 bp, Artificial Sequence
  VB-111 entire construct
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtacgtacgtgtacttctgatcggcgatactagggagataaggatgtgcctgacaaaaccacattgttgttgttatcattattatttagttttccttccttgctaactcctgacggaatctttctcacctcaaatgcgaagtactttagtttagaaaagacttggtggaaggggtggtggtggaaaagtagggtgatcttccaaactaatctggttccccgcccgccccagtagctgggattcaagagcgaagagtggggatcgtccccttgtttgatcagaaagacataaaaggaaaatcaagtgaacaatgatcagccccacctccaccccacccccctgcgcgcgcacaatacaatctatttaattgtacttcatacttttcattccaatggggtgactttgcttctggagaaactcttgattcttgaactctggggctggcagctagcctccagaagcaaagtcaccccattggaatgaaaagtatgaagtacaatgaaaagtatgaagtactggctccagaagcaaagtcaccctccagaagcaaagtcaccccattggaatgaaaagtatgaagtacgctagcaaaaggggaagcgggctgctgctctctgcaggttctgcagcggtctctgtctagtgggtgttttctttttcttagccctgcccctggattgtcagacggcgggcgtctgcctctgaagttagccgtgatttcctctagagccgggtcttatctctggctgcacgttgcctgtgggtgactaatcacacaataacattgtttagggctggaatgaagtcagagctgtttacccccactctataggggttcaatataaaaaggcggcggagaactgtccgagtcagaagcgttcctgcaccggcgctgagagcctgacccggtctgctccgctgtccttgcgcgctgcctcccggctgcccgcgacgctttcgccccagtggaagggccacttgctgcggccgctaattctgcagatcgggatccggcatgggcctctccaccgtgcctgacctgctgctgccgctggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccaagcttaggatccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctagctcgagcatgcatctaggcggccgcatggcagaaattcgcgaattcgctagcgttaacggatcctctagacgagatccgaacttgtttattgcagcttataatggttacaaataaagcaatagcatcacaaatttcacaaataaagcatttttttcactgcattctagttgtggtttgtccaaactcatcaatgtatcttatcatgtctagatctgtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaatcggggttggggttattctctgtcttgtgattctctttattcttatactaacgcttctctgcctaaggctcgccgcctgctgtgtgcacatttgcatttattgtcagctttttaaacgctggggtcgccacccaagatgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaatttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgctttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaacgcaatagatgccatgaaccacccaactttccccgcgcccgctatgcttccactgcaacaagttgttgccggcggctttgtcccagccaatcagcctcgcccaccttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 20-21 - skipped (2 entries)

SEQ ID NO: 22 - DNA, 19 bp, Artificial Sequence
  Endothelial Cell-Specific Enhancer Element
ggtgactttgcttctggag

SEQ ID NO: 23 - DNA, 19 bp, Artificial Sequence
  Endothelial Cell-Specific Enhancer Element
ctccagaagcaaagtcacc

SEQ ID NO: 24 - DNA, 19 bp, Artificial Sequence
  Endothelial Cell-Specific Enhancer Element
gtacttcatacttttcatt

SEQ ID NO: 25 - DNA, 19 bp, Artificial Sequence
  Endothelial Cell-Specific Enhancer Element
aatgaaaagtatgaagtac

SEQ ID NO: 26 - DNA, 6 bp, Artificial Sequence
  Hypoxia Response element
gcacgt

SEQ ID NO: 27 - protein, 40 aa, Artificial Sequence
  peptide linker
GGGSGGGSGGGSGGGSGGGSGGGSGGGSGGGSGGGSGGGS

SEQ ID NO: 28 - protein, 10 aa, Artificial Sequence
  VH CDR1
GYTFTNYGMN

SEQ ID NO: 29 - protein, 17 aa, Artificial Sequence
  VH CDR2
WINTYTGEPTYAADFKR

SEQ ID NO: 30 - protein, 14 aa, Artificial Sequence
  VH CDR3
YPHYYGSSHWYFDV

SEQ ID NO: 31 - protein, 11 aa, Artificial Sequence
  VL CDR1
SASQDISNYLN

SEQ ID NO: 32 - protein, 7 aa, Artificial Sequence
  VL CDR2
FTSSLHS

SEQ ID NO: 33 - protein, 9 aa, Artificial Sequence
  VL CDR3
QQYSTVPWT

(19) United States
(12) Patent Application Publication
(10) Pub. No.: US 2021/0322574 A1
(43) Pub. Date: Oct. 21, 2021

(54) DIAGNOSTIC METHODS FOR ANTI-ANGIOGENIC AGENT THERAPY

(71) Applicant: VASCULAR BIOGENICS LTD., Modiin (IL)

(72) Inventors: Itzhak MENDEL, Rechovot (IL); Eyal BREITBART, Hashmonaim (IL)

(73) Assignee: VASCULAR BIOGENICS LTD., Modiin (IL)

(21) Appl. No.: 16/756,216

(22) PCT Filed: Oct. 22, 2018
(86) PCT No.: PCT/IB2018/058218

§ 371 Date: Apr. 15, 2020

Related U.S. Application Data
(60) Provisional application No. 62/575,193, filed on Oct. 20, 2017.
(60) Provisional application No. 62/722,671, filed on Aug. 24, 2018.

(51) Int. Cl. A61K 48/00; C12N 15/86; A61K 45/06

(52) U.S. Cl. CPC A61K 48/0025; A61K 45/06; C12N 15/86

(57) ABSTRACT

The disclosure provides methods of treating a tumor in a subject who is capable of exhibiting a change in at least one plasma biomarker or cell surface biomarker after administration of at least one priming dose of a vector which comprises a Fas-chimera gene operably linked to an endothelial cell-specific promoter. Also provided are methods of identifying a responder to a Fas-chimera gene therapy in a subject having a tumor.

### BACKGROUND OF THE DISCLOSURE

Angiogenesis is a common and major feature of several pathologies. Among these are diseases in which the angiogenesis can improve the disease condition (such as ischemic heart disease) and diseases in which the excessive angiogenesis is a part of the pathology and thus should be eliminated. These latter diseases include diabetes (diabetic retinopathy), cardiovascular diseases (atherosclerosis), chronic inflammation (rheumatoid arthritis), and cancer. Angiogenesis occurs in tumors and permits their growth, invasion and metastasis. In 1971, Folkman proposed that tumor growth and metastases are angiogenesis dependent, and thus inhibiting angiogenesis can be a strategy to arrest tumor growth.

There are several molecules involved in angiogenesis, from transcription factors to growth factors. Hypoxia is an important environmental factor that leads to neovascularization, and it induces release of several cytokines that are pro-angiogenic factors. Among them are vascular endothelial growth factors (VEGF) and their receptors, members of the angiopoietin family, basic fibroblast growth factor, and endothelin-1 (ET-1). These factors are involved in induction of angiogenesis through activation, proliferation and migration of endothelial cells.

Recombinant forms of endogenous inhibitors of angiogenesis were tested for the treatment of cancer. The potential pharmacokinetic, biotechnological and economic drawbacks of chronic delivery of these recombinant inhibitors have led scientists to develop other approaches.

The development of the anti-VEGF monoclonal antibody bevacizumab has validated an antiangiogenic approach as a complementary therapeutic modality to chemotherapy. Several small molecule inhibitors, including second-generation multi-targeted tyrosine kinase inhibitors, have also shown promise as antiangiogenic agents for cancer.

However, the potential pharmacokinetic and economic drawbacks of chronic delivery of recombinant inhibitors, antibodies, and small molecules, as well as the limited activity manifested when applied as monotherapy have led scientists to evaluate antiangiogenic gene therapy. Gene therapy is an emerging modality for treating inherited and acquired human diseases. However, there are a number of obstacles limiting successful gene therapy, including duration of expression, induction of the immune response, cytotoxicity of the vectors and tissue specificity. Two general strategies for the cancer gene therapy were proposed: tumor directed or systemic gene therapy. The lack of success in targeting gene therapy products to cancerous cells or their environment by systemic treatments caused most therapies to be administered to the tumor itself.

### BRIEF SUMMARY OF THE DISCLOSURE

The present disclosure is directed to a method of treating a tumor in a subject who is capable of exhibiting a change in at least one plasma biomarker or cell surface biomarker after administration of at least one priming dose of a vector which comprises a Fas-chimera gene operably linked to an endothelial cell-specific promoter, the method comprising administering at least one therapeutically effective dose of the vector to the subject after the subject exhibits a change in at least one plasma biomarker or cell surface biomarker after the administration of the priming dose. The disclosure is further directed to a method of treating a tumor in a subject in need thereof comprising administering to the subject at least one priming dose of a vector which comprises a Fas-chimera gene operably linked to an endothelial cell-specific promoter and administering to the subject a therapeutically effective dose of the vector, wherein the subject has a change in at least one plasma biomarker or cell surface biomarker after the administration of the priming dose. The disclosure also provides a method of treating a tumor in a subject in need thereof, the method comprising: (a) administering to the subject at least one priming dose of a vector which comprises a Fas-chimera gene operably linked to an endothelial cell-specific promoter; (b) determining the subject as having a change in at least one plasma biomarker or cell surface biomarker as a result of the administration of the priming dose in (a); and (c) administering a therapeutically effective dose of the vector to the subject having a change in at least one plasma biomarker or cell surface biomarker in (b). The disclosure further provides a method of treating a tumor in a subject in need thereof, the method comprising: (a) administering to the subject at least one priming dose of a vector which comprises a Fas-chimera gene operably linked to an endothelial cell-specific promoter; (b) measuring serum levels of at least one plasma biomarker or cell surface biomarker of the subject after the administration of the priming dose; (c) determining the subject as having a change in at least one plasma biomarker or cell surface biomarker as a result of the administration of the priming dose in (a); and (d) administering a therapeutically effective dose of the vector to the subject having a change in at least one plasma biomarker or cell surface biomarker in (c).

The disclosure further provides a method of identifying a responder to a Fas-chimera gene therapy, the method comprising administering to a subject having a tumor at least one priming dose of a vector which comprises a Fas-chimera gene operably linked to an endothelial cell-specific promoter, wherein the subject exhibits a change in at least one plasma biomarker or cell surface biomarker after the administration of the priming dose. In another aspect, the disclosure provides a method for identifying a responder to a Fas-chimera gene therapy, the method comprising administering to a subject having a tumor at least one priming dose of a vector which comprises a Fas-chimera gene operably linked to an endothelial cell-specific promoter and administering to the subject a therapeutically effective dose of the vector, wherein the subject exhibits a change in at least one plasma biomarker or cell surface biomarker after the administration of the priming dose. In another aspect, the disclosure provides a method of identifying a responder to a Fas-chimera gene therapy, the method comprising: (a) administering to a subject at least one priming dose of a vector which comprises a Fas-chimera gene operably linked to an endothelial cell-specific promoter; (b) identifying the subject as having a change in at least one plasma biomarker or cell surface biomarker as a result of the administration of the priming dose in (a); and (c) administering a therapeutically effective dose of the vector to the subject having a change in at least one plasma biomarker or cell surface biomarker in (b).

In some aspects of the disclosure, the at least one plasma biomarker or cell surface biomarker is selected from the group consisting of MCP-1, MIP-1, MIP-2, MIP-1α, MIP-1β, MIG, RANTES, IP-10, IL-1α, IL-113, IL-2, IL-3, IL-4, IL-5, IL-6, IL-8, IL-9, IL-10, IL-12, IL-13, IL-15, IL-17a, IL-22, IL-23, IL-35, LIF, TNF-α, TNF-β, TGF-β1, VEGF, G-CSF, GM-CSF, IFN-α, IFN-β, IFN-γ, M-CSF, IL-1Ra, eotaxin, CA-125, and a combination thereof. In one aspect, the change in plasma biomarker or cell surface marker is an increase in the level of at least one plasma biomarker or cell surface marker. In some aspects, the plasma biomarker or cell surface marker that exhibits an increase in the level after the administration of the priming dose comprises at least one of the markers selected from the group consisting of IL-6, MCP-1, MIP-2, MIG, RANTES, MIP-1α, MIP-113, IP-10, IL-2, IL-10, LIF, TNF-α, TGF-β1, VEGF, IL-17α, G-CSF, GM-CSF, IFN-γ, IL-1α, IL-1β, IL-12, IL-13, IL-15, IL-9, IL-22, IL-23, IL-35, M-CSF, IL-1Ra, and a combination thereof. In a particular aspect, the plasma biomarker or cell surface maker comprises TNF-α, IL-17α, MIP-1α, and IL-1Ra.

In some aspects, the change in plasma biomarker or cell surface marker is a decrease in the level at least one plasma biomarker or cell surface marker. In some aspects, the plasma biomarker or cell surface marker that exhibits a decrease in the level comprises at least one of the markers selected from IL-10, IL-4, IL-3, IL-5, IL-13, IL-2, LIF, TNF-α, CA-125, and a combination thereof.

In some aspects of the disclosure, the priming dose is identical to the therapeutically effective dose. In other aspects, the priming dose is lower than the therapeutically effective dose. In other aspects, the priming dose is higher than the therapeutically effective dose. In some aspects, the priming dose is administered more than once.

In some aspects, the vector comprising a Fas-chimera gene encodes a polypeptide comprising an extracellular domain of a TNF Receptor 1 (TNFR1) polypeptide fused to a transmembrane domain and an intracellular domain of a Fas polypeptide. In some aspects, the extracellular domain of the TNFR1 comprises an amino acid sequence at least about 70%, 80%, 90%, 95%, 96%, 97%, 98%, 99%, or 100% identical to SEQ ID NO: 4, wherein the extracellular domain of the TNFR1 is capable of binding to TNF-α. In some aspects, the trans-membrane domain and the intracellular domain of the Fas polypeptide comprises an amino acid sequence at least about 70%, 80%, 90%, 95%, 96%, 97%, 98%, 99%, or 100% identical to SEQ ID NO: 8, wherein the trans-membrane domain and the intracellular domain of the Fas polypeptide is capable of inducing Fas mediated apoptosis. In some aspects of the disclosure, the Fas-chimera gene comprises a first nucleotide sequence, which is at least 70%, 80%, 90%, 95%, 96%, 97%, 98%, 99%, or 100% identical to SEQ ID NO: 3, and a second nucleotide sequence, which is at least about 70%, 80%, 90%, 95%, 96%, 97%, 98%, 99%, or 100% identical to SEQ ID NO: 7.

In some aspects, the endothelial cell-specific promoter of the vector comprises a PPE-1 promoter. In some aspects, the endothelial cell-specific promoter further comprises a cis-acting regulatory element. In some aspects, the cis-acting regulatory element comprises a nucleotide sequence at least about 70%, 80%, 90%, 95%, 96%, 97%, 98%, 99%, or 100% identical to SEQ ID NO: 15 or SEQ ID NO: 16. In some aspects, the cis-acting regulatory element comprises SEQ ID NO: 11 or SEQ ID NO: 12. In some aspects, the cis-acting regulatory element further comprises SEQ ID NO: 13 or SEQ ID NO: 14.

In some aspects of the disclosure, the endothelial cell-specific promoter is a PPE-1-3X promoter. In some aspects, the PPE-1-3X promoter comprises a nucleotide sequence at least about 70%, 80%, 90%, 95%, 96%, 97%, 98%, 99%, or 100% identical to SEQ ID NO: 18, wherein the PPE-1-3X promoter is capable of directing the Fas-chimera gene expression in endothelial cells. In some aspects of the disclosure, the vector does not contain an E1 region of an adenovirus.

In some embodiments of the disclosure, the priming dose of the vector is administered at an amount of less than about 1×10¹⁵, less than about 1×10¹⁴, less than about 5×10¹³, less than about 4×10¹³, less than about 3×10¹³, less than about 2×10¹³, less than about 1×10¹³, less than about 9×10¹², less than about 8×10¹², less than about 7×10¹², less than about 6×10¹², less than about 5×10¹², less than about 4×10¹², less than about 3×10¹², less than about 2×10¹², less than about 1×10¹², less than about 9×10¹¹, less than about 8×10¹¹, less than about 7×10¹¹, less than about 6×10¹¹, less than about 5×10¹¹, less than about 4×10¹¹, less than about 3×10¹¹, less than about 2×10¹¹, less than about 1×10¹¹, less than about 9×10¹⁰, less than about 8×10¹⁰, less than about 7×10¹⁰, less than about 6×10¹⁰, less than about 5×10¹⁰, less than about 4×10¹⁰, less than about 3×10¹⁰, less than about 2×10¹⁰, or less than about 1×10¹⁰ virus particles.

In some embodiments of the disclosure, the therapeutically effective dose of the vector is administered at an amount of at least about 1×10¹⁵, at least about 1×10¹⁴, at least about 5×10¹³, at least about 4×10¹³, at least about 3×10¹³, at least about 2×10¹³, at least about 1×10¹³, at least about 9×10¹², at least about 8×10¹², at least about 7×10¹², at least about 6×10¹², at least about 5×10¹², at least about 4×10¹², at least about 3×10¹², at least about 2×10¹², at least about 1×10¹², at least about 9×10¹¹, at least about 8×10¹¹, at least about 7×10¹¹, at least about 6×10¹¹, at least about 5×10¹¹, at least about 4×10¹¹, at least about 3×10¹¹, at least about 2×10¹¹, at least about 1×10¹¹, at least about 9×10¹⁰, at least about 8×10¹⁰, at least about 7×10¹⁰, at least about 6×10¹⁰, at least about 5×10¹⁰, at least about 4×10¹⁰, at least about 3×10¹⁰, at least about 2×10¹⁰, or at least about 1×10¹⁰ virus particles.

In some aspects of the disclosure, the therapeutically effective dose of the vector is repeatedly administered. The therapeutically effective dose of the vector can be repeatedly administered every day, once in about 2 days, once in about 3 days, once in about 4 days, once in about 5 days, once in about 6 days, once in about 7 days, once in about 2 weeks, once in about 3 weeks, once in about 4 weeks, once in about 5 weeks, once in about 6 weeks, once in about 7 weeks, once in about 2 months, or once in about 6 months.

In some aspects of the disclosure, administration of the therapeutically effective dose of the vector reduces angiogenesis in the tumor of the subject. In some aspects, the tumor is a solid tumor. In some aspects, the tumor is a metastatic tumor.

In some aspects of the disclosure, the vector is an adenovirus vector. In one aspect, the adenovirus vector is adenovirus serotype 5. In some aspects, the vector comprises, consists of, or consists essentially of SEQ ID NO: 19. In some aspects, the vector is an isolated virus having European Collection of Cell Cultures (ECACC) Accession Number 13021201.

In some aspects of the disclosure, the endothelial cell-specific promoter of the vector comprises a hypoxia response element.

In one embodiment, the priming dose of the vector is administered at an amount of at least about 1×10¹¹ virus particles. In another embodiment, the priming dose of the vector is administered at an amount of at least about 1×10¹² virus particles. In another embodiment, the priming dose of the vector is administered at an amount of at least about 1×10¹³ virus particles. In another embodiment, the priming dose of the vector is administered at an amount of at least about 1×10¹⁴ virus particles.

In one embodiment, the therapeutically effective dose of the vector is at least about 1×10¹¹ virus particles. In another embodiment, the therapeutically effective dose of the vector is at least about 1×10¹² virus particles. In another embodiment, the therapeutically effective dose of the vector is at least about 1×10¹³ virus particles. In another embodiment, the therapeutically effective dose of the vector is at least about 1×10¹⁴ virus particles.

In one embodiment, the method further comprises administering to the subject an effective amount of a VEGF antagonist. In some aspects, the VEGF antagonist is administered prior to, concurrently with, or after the administration of the vector. In one embodiment, the VEGF antagonist is selected from the group consisting of bevacizumab, ranibizumab, VGX-100, r84, aflibercept, IMC-18F1, IMC-1C11, and ramucirumab. In a particular embodiment, the VEGF antagonist is bevacizumab.

In another embodiment of the present disclosure, bevacizumab is administered at an effective amount of equal to or less than about 15 mg/kg, 14 mg/kg, 13 mg/kg, 12 mg/kg, 11 mg/kg, 10 mg/kg, 9 mg/kg, 8 mg/kg, 7 mg/kg, 6 mg/kg, 5 mg/kg, 4 mg/kg, 3 mg/kg, 2 mg/kg, or 1 mg/kg. In some embodiments, the bevacizumab is repeatedly administered. In some embodiments, the bevacizumab is repeatedly administered once in about 7 days, once in about 2 weeks, once in about 3 weeks, once in about 4 weeks, once in about 2 months, once in about 3 months, once in about 4 months, once in about 5 months, or once in about 6 months.

In another embodiment of the present disclosure, the priming dose and therapeutically effective dose of the vector is administered at an effective amount of 3×10¹² to 1×10¹³ virus particles and bevacizumab is administered at an effective amount of 5 mg/kg to 15 mg/kg.

In some embodiments, the priming dose of the vector is repeatedly administered. In further aspects, the therapeutically effective dose of the vector is repeatedly administered. The priming dose and the therapeutically effective dose of the vector can be repeatedly administered every day, once in about 2 days, once in about 3 days, once in about 4 days, once in about 5 days, once in about 6 days, once in about 7 days, once in about 2 weeks, once in about 3 weeks, once in about 4 weeks, once in about 5 weeks, once in about 6 weeks, once in about 7 weeks, once in about 2 months, or once in about 6 months. In certain embodiments, the bevacizumab is repeatedly administered. In another embodiment of the disclosure, the bevacizumab is repeatedly administered once in about 7 days, once in about 2 weeks, once in about 3 weeks, once in about 4 weeks, once in about 2 months, once in about 3 months, once in about 4 months, once in about 5 months, or once in about 6 months.

In another embodiment, the subject exhibits a fever after the administration of the priming dose.

In some embodiments, the method further comprises administering to the subject an effective amount of one or more chemotherapeutic agents. In some aspects, the one or more chemotherapeutic agents are administered prior to, concurrently with, or after the administration of the vector. In some embodiments, the one or more chemotherapeutic agents are selected from the group consisting of altretamine, raltritrexed, topotecan, paclitaxel, docetaxel, cisplatin, carboplatin, oxaliplatin, liposomal doxorubicin, gemcitabine, cyclophosphamide, vinorelbine, ifosfamide, etoposide, altretamine, capecitabine, irinotecan, melphalan, pemetrexed, bevacizumab, and albumin bound paclitaxel. In a particular aspect, the chemotherapeutic agent is paclitaxel.

In other embodiments of the present disclosure, paclitaxel is administered at an effective amount of at least about 10 mg/m², at least about 20 mg/m², at least about 30 mg/m², at least about 40 mg/m², at least about 50 mg/m², at least about 60 mg/m², at least about 70 mg/m², at least about 80 mg/m², at least about 90 mg/m², or at least about 100 mg/m². In some aspects, the effective amount of paclitaxel is about 10 mg/m² to about 100 mg/m², about 20 mg/m² to about 90 mg/m², about 30 mg/m² to about 90 mg/m²; about 40 mg/m² to about 90 mg/m²; about 50 mg/m² to about 90 mg/m², about 60 mg/m² to about 90 mg/m², or about 70 mg/m² to about 90 mg/m². In a particular aspect, the effective amount of paclitaxel is about 80 mg/m².

In some embodiments, the paclitaxel is repeatedly administered. In some embodiments, the paclitaxel is repeatedly administered every day, every two days, every three days, every four days, every five days, every six days, every seven days, every eight days, every nine days, or every ten days.

In other embodiments of the present disclosure, the priming dose and therapeutically effective dose of the vector are administered at an effective amount of 3×10¹² to 1×10¹³ virus particles and paclitaxel is administered at an effective amount of 70 mg/m² to about 90 mg/m².

In some aspects, the therapeutically effective dose of the vector is repeatedly administered. The therapeutically effective dose of the vector can be repeatedly administered every day, once in about 2 days, once in about 3 days, once in about 4 days, once in about 5 days, once in about 6 days, once in about 7 days, once in about 2 weeks, once in about 3 weeks, once in about 4 weeks, once in about 5 weeks, once in about 6 weeks, once in about 7 weeks, once in about 2 months, or once in about 6 months. In certain embodiments, the paclitaxel is repeatedly administered. In another embodiment of the disclosure, the paclitaxel is repeatedly administered every day, every two days, every three days, every four days, every five days, every six days, every seven days, every eight days, every nine days, or every ten days.

In another embodiment, the subject exhibits a fever after the administration of the priming dose.

The present disclosure further provides a method of treating a tumor in a subject who is capable of exhibiting a febrile body temperature after administration of at least one priming dose of a vector which comprises a Fas-chimera gene operably linked to an endothelial cell-specific promoter, the method comprising administering a therapeutically effective dose of the vector to the subject after the subject exhibits a febrile body temperature after the administration of the priming dose. The present disclosure also provides a method of treating a tumor in a subject in need thereof comprising administering to the subject at least one priming dose of a vector which comprises a Fas-chimera gene operably linked to an endothelial cell-specific promoter and administering to the subject a therapeutically effective dose of the vector, wherein the subject has a febrile body temperature after the administration of the priming dose. The disclosure also provides a method of treating a tumor in a subject in need thereof, the method comprising: (a) administering to the subject at least one priming dose of a vector which comprises a Fas-chimera gene operably linked to an endothelial cell-specific promoter; (b) determining the subject as having a febrile body temperature as a result of the administration of the priming dose in (a); and (c) administering a therapeutically effective dose of the vector to the subject having a febrile body temperature in (b). The present disclosure also provides a method of treating a tumor in a subject in need thereof, the method comprising: (a) administering to the subject at least one priming dose of a vector which comprises a Fas-chimera gene operably linked to an endothelial cell-specific promoter; (b) measuring the body temperature of the subject after the administration of the priming dose; (c) determining the subject as having a febrile body temperature as a result of the administration of the priming dose in (a); and (d) administering a therapeutically effective dose of the vector to the subject having a febrile body temperature in (c).

The present disclosure is also directed to a method for identifying a responder to a Fas-chimera gene therapy, the method comprising administering to a subject having a tumor at least one priming dose of a vector which comprises a Fas-chimera gene operably linked to an endothelial cell-specific promoter, wherein the subject exhibits a febrile body temperature after the administration of the priming dose. The disclosure provides a method for identifying a responder to a Fas-chimera gene therapy, the method comprising administering to a subject having a tumor at least one priming dose of a vector which comprises a Fas-chimera gene operably linked to an endothelial cell-specific promoter and administering to the subject a therapeutically effective dose of the vector, wherein the subject exhibits a febrile body temperature after the administration of the priming dose. The disclosure also provides a method for identifying a responder to a Fas-chimera gene therapy, the method comprising: (a) administering to a subject at least one priming dose of a vector which comprises a Fas-chimera gene operably linked to an endothelial cell-specific promoter; (b) identifying the subject as having a febrile body temperature as a result of the administration of the priming dose in (a); and (c) administering a therapeutically effective dose of the vector to the subject having a febrile body temperature in (b).

In some aspects, the priming dose used in the methods is identical to the therapeutically effective dose. In some aspects, the priming dose used in the methods is lower than the therapeutically effective dose. In some aspects, the priming dose used in the methods is higher than the therapeutically effective dose. In some aspects, the priming dose used in the methods is administered more than once.

### EMBODIMENTS

1. A method of treating a tumor in a subject who is capable of exhibiting a change in at least one plasma biomarker or cell surface biomarker after administration of at least one priming dose of a vector which comprises a Fas-chimera gene operably linked to an endothelial cell-specific promoter, the method comprising administering a therapeutically effective dose of the vector to the subject after the subject exhibits a change in at least one plasma biomarker or cell surface biomarker after the administration of the priming dose.

2. A method of treating a tumor in a subject in need thereof comprising administering to the subject at least one priming dose of a vector which comprises a Fas-chimera gene operably linked to an endothelial cell-specific promoter and administering to the subject a therapeutically effective dose of the vector, wherein the subject has a change in at least one plasma biomarker or cell surface biomarker after the administration of the priming dose.

3. A method of treating a tumor in a subject in need thereof, the method comprising: (a) administering to the subject at least one priming dose of a vector which comprises a Fas-chimera gene operably linked to an endothelial cell-specific promoter; (b) determining the subject as having a change in at least one plasma biomarker or cell surface biomarker as a result of the administration of the priming dose in (a); and (c) administering a therapeutically effective dose of the vector to the subject having a change in at least one plasma biomarker or cell surface biomarker in (b).

4. A method of treating a tumor in a subject in need thereof, the method comprising: (a) administering to the subject at least one priming dose of a vector which comprises a Fas-chimera gene operably linked to an endothelial cell-specific promoter; (b) measuring serum levels of at least one plasma biomarker or cell surface biomarker of the subject after the administration of the priming dose; (c) determining the subject as having a change in at least one plasma biomarker or cell surface biomarker as a result of the administration of the priming dose in (a); and (d) administering a therapeutically effective dose of the vector to the subject having a change in at least one plasma biomarker or cell surface biomarker in (c).

5. A method for identifying a responder to a Fas-chimera gene therapy, the method comprising administering to a subject having a tumor at least one priming dose of a vector which comprises a Fas-chimera gene operably linked to an endothelial cell-specific promoter, wherein the subject exhibits a change in at least one plasma biomarker or cell surface biomarker after the administration of the priming dose.

6. A method for identifying a responder to a Fas-chimera gene therapy, the method comprising administering to a subject having a tumor at least one priming dose of a vector which comprises a Fas-chimera gene operably linked to an endothelial cell-specific promoter and administering to the subject a therapeutically effective dose of the vector, wherein the subject exhibits a change in at least one plasma biomarker or cell surface biomarker after the administration of the priming dose.

7. A method for identifying a responder to a Fas-chimera gene therapy, the method comprising: (a) administering to a subject at least one priming dose of a vector which comprises a Fas-chimera gene operably linked to an endothelial cell-specific promoter; (b) identifying the subject as having a change in at least one plasma biomarker or cell surface biomarker as a result of the administration of the priming dose in (a); and (c) administering a therapeutically effective dose of the vector to the subject having a change in at least one plasma biomarker or cell surface biomarker in (b).

8. The method of any one of embodiments 1 to 7, wherein the at least one plasma biomarker or cell surface biomarker is selected from the group consisting of MCP-1, MIP-1, MIP-2, MIP-1α, MIP-1β, MIG, RANTES, IP-10, IL-1α, IL-1β, IL-2, IL-3, IL-4, IL-5, IL-6, IL-8, IL-9, IL-10, IL-12, IL-13, IL-15, IL-17α, IL-22, IL-23, IL-35, LIF, TNF-α, TNF-β, TGF-β1, VEGF, G-CSF, GM-CSF, IFN-α, IFN-β, IFN-γ, M-CSF, IL-1Ra, eotaxin, CA-125, and a combination thereof.

9. The method of any one of embodiments 1 to 7, wherein the change in plasma biomarker or cell surface marker is an increase in the level of at least one plasma biomarker or cell surface marker.

10. The method of embodiment 9, wherein the plasma biomarker or cell surface marker that exhibits an increase in the level after the administration of the priming dose comprises at least one of the markers selected from the group consisting of IL-6, MCP-1, MIP-2, MIG, RANTES, MIP-1α, MIP-1β, IP-10, IL-2, IL-10, LIF, TNF-α, TGF-β1, VEGF, IL-17α, G-CSF, GM-CSF, IFN-γ, IL-1α, IL-1β, IL-12, IL-13, IL-15, IL-9, IL-22, IL-23, IL-35, M-CSF, IL-1Ra, and a combination thereof.

11. The method of embodiments 9 or 10, wherein the plasma biomarker or cell surface maker comprises TNF-α, IL-17α, MIP-1α, and IL-1Ra.

12. The method of any one of embodiments 1 to 7, wherein the change in plasma biomarker or cell surface marker is a decrease in the level at least one plasma biomarker or cell surface marker.

13. The method of embodiment 12, wherein the plasma biomarker or cell surface marker that exhibits a decrease in the level comprises at least one of the markers selected from IL-10, IL-4, IL-3, IL-5, IL-13, IL-2, LIF, TNF-α, CA-125, and a combination thereof.

14. The method of any one of embodiments 1 to 13, wherein the priming dose is identical to the therapeutically effective dose.

15. The method of any one of embodiments 1 to 13, wherein the priming dose is lower than the therapeutically effective dose.

16. The method of any one of embodiments 1 to 13, wherein the priming dose is higher than the therapeutically effective dose.

17. The method of any one of embodiments 1 to 16, wherein the priming dose is administered more than once.

18. The method of any one of embodiments 1 to 17, wherein the Fas-chimera gene encodes a polypeptide comprising an extracellular domain of a TNF Receptor 1 (TNFR1) polypeptide fused to a trans-membrane domain and an intracellular domain of a Fas polypeptide.

19. The method of embodiment 18, wherein the extracellular domain of the TNFR1 comprises an amino acid sequence at least about 70%, 80%, 90%, 95%, 96%, 97%, 98%, 99%, or 100% identical to SEQ ID NO: 4, wherein the extracellular domain of the TNFR1 is capable of binding to TNF-α.

20. The method of embodiment 19, wherein the trans-membrane domain and the intracellular domain of the Fas polypeptide comprises an amino acid sequence at least about 70%, 80%, 90%, 95%, 96%, 97%, 98%, 99%, or 100% identical to SEQ ID NO: 8, wherein the trans-membrane domain and the intracellular domain of the Fas polypeptide is capable of inducing Fas mediated apoptosis.

21. The method of any of embodiments 1 to 20, wherein the Fas-chimera gene comprises a first nucleotide sequence, which is at least 70%, 80%, 90%, 95%, 96%, 97%, 98%, 99%, or 100% identical to SEQ ID NO: 3, and a second nucleotide sequence, which is at least about 70%, 80%, 90%, 95%, 96%, 97%, 98%, 99%, or 100% identical to SEQ ID NO: 7.

22. The method of any one of embodiments 1 to 21, wherein the endothelial cell-specific promoter comprises a PPE-1 promoter.

23. The method of any one of embodiments 1 to 22, wherein the endothelial cell-specific promoter further comprises a cis-acting regulatory element.

24. The method of embodiment 23, wherein the cis-acting regulatory element comprises a nucleotide sequence at least about 70%, 80%, 90%, 95%, 96%, 97%, 98%, 99%, or 100% identical to SEQ ID NO: 15 or SEQ ID NO: 16.

25. The method of embodiment 24, wherein the cis-acting regulatory element comprises SEQ ID NO: 11 or SEQ ID NO: 12.

26. The method of embodiment 25, wherein the cis-acting regulatory element further comprises SEQ ID NO: 13 or SEQ ID NO: 14.

27. The method of any one of embodiments 1 to 26, wherein the endothelial cell-specific promoter is a PPE-1-3X promoter.

28. The method of embodiment 27, wherein the PPE-1-3X promoter comprises a nucleotide sequence at least about 70%, 80%, 90%, 95%, 96%, 97%, 98%, 99%, or 100% identical to SEQ ID NO: 18, wherein the PPE-1-3X promoter is capable of directing the Fas-chimera gene expression in endothelial cells.

29. The method of any one of embodiments 1 to 28, wherein the vector does not contain an E1 region of an adenovirus.

30. The method of any one of embodiments 1 to 29, wherein the priming dose of the vector is administered at an amount of less than about 1×10¹⁵, less than about 1×10¹⁴, less than about 5×10¹³, less than about 4×10¹³, less than about 3×10¹³, less than about 2×10¹³, less than about 1×10¹³, less than about 9×10¹², less than about 8×10¹², less than about 7×10¹², less than about 6×10¹², less than about 5×10¹², less than about 4×10¹², less than about 3×10¹², less than about 2×10¹², less than about 1×10¹², less than about 9×10¹¹, less than about 8×10¹¹, less than about 7×10¹¹, less than about 6×10¹¹, less than about 5×10¹¹, less than about 4×10¹¹, less than about 3×10¹¹, less than about 2×10¹¹, less than about 1×10¹¹, less than about 9×10¹⁰, less than about 8×10¹⁰, less than about 7×10¹⁰, less than about 6×10¹⁰, less than about 5×10¹⁰, less than about 4×10¹⁰, less than about 3×10¹⁰, less than about 2×10¹⁰, or less than about 1×10¹⁰ virus particles.

31. The method of any one of embodiments 1 to 29, wherein the therapeutically effective dose of the vector is administered at an amount of at least about 1×10¹⁵, at least about 1×10¹⁴, at least about 5×10¹³, at least about 4×10¹³, at least about 3×10¹³, at least about 2×10¹³, at least about 1×10¹³, at least about 9×10¹², at least about 8×10¹², at least about 7×10¹², at least about 6×10¹², at least about 5×10¹², at least about 4×10¹², at least about 3×10¹², at least about 2×10¹², at least about 1×10¹², at least about 9×10¹¹, at least about 8×10¹¹, at least about 7×10¹¹, at least about 6×10¹¹, at least about 5×10¹¹, at least about 4×10¹¹, at least about 3×10¹¹, at least about 2×10¹¹, at least about 1×10¹¹, at least about 9×10¹⁰, at least about 8×10¹⁰, at least about 7×10¹⁰, at least about 6×10¹⁰, at least about 5×10¹⁰, at least about 4×10¹⁰, at least about 3×10¹⁰, at least about 2×10¹⁰, or at least about 1×10¹⁰ virus particles.

32. The method of any one of embodiments 1 to 31 wherein the therapeutically effective dose of the vector is repeatedly administered.

33. The method of any one of embodiments 1 to 32, wherein the therapeutically effective dose of the vector is repeatedly administered every day, once in about 2 days, once in about 3 days, once in about 4 days, once in about 5 days, once in about 6 days, once in about 7 days, once in about 2 weeks, once in about 3 weeks, once in about 4 weeks, once in about 5 weeks, once in about 6 weeks, once in about 7 weeks, once in about 2 months, or once in about 6 months.

34. The method of any one of embodiments 1 to 33, wherein administration of the therapeutically effective dose of the vector reduces angiogenesis in the tumor of the subject.

35. The method of any one of embodiments 1 to 34, wherein the tumor is a solid tumor.

36. The method of any one of embodiments 1 to 35, wherein the tumor is a metastatic tumor.

37. The method of any one of embodiments 1 to 36 wherein the vector is an adenovirus vector.

38. The method of embodiment 37, wherein the adenovirus vector is adenovirus serotype 5.

39. The method of any one of embodiments 1 to 38, wherein the vector comprises, consists of, or consists essentially of SEQ ID NO: 19.

40. The method of any one of embodiments 1 to 39, wherein the vector is an isolated virus having European Collection of Cell Cultures (ECACC) Accession Number 13021201.

41. The method of any one of embodiments 1 to 40, wherein the promoter comprises a hypoxia response element.

42. The method of any one of embodiments 1 to 41, wherein the therapeutically effective dose of the vector is about 1×10¹¹, about 1×10¹², about 1×10¹³, or about 1×10¹⁴ virus particles.

43. The method of any one of embodiments 1 to 42, further comprising administering to the subject an effective amount of a VEGF antagonist.

44. The method of embodiment 43, wherein the VEGF antagonist is administered prior to, concurrently with, or after the administration of the priming dose of the vector.

45. The method of embodiment 44, wherein the VEGF antagonist is administered prior to, concurrently with, or after the administration of the therapeutically effective dose of the vector.

46. The method of any one of claims 44 to 45, wherein the VEGF antagonist is selected from the group consisting of bevacizumab, ranibizumab, VGX-100, r84, aflibercept, IMC-18F1, IMC-1C11, ramucirumab, and any combination thereof.

47. The method of any one of embodiments 43 to 46, wherein the VEGF antagonist is bevacizumab.

48. The method of embodiment 47, wherein the bevacizumab is repeatedly administered.

49. The method of embodiment 48, wherein the bevacizumab is repeatedly administered once in about 7 days, once in about 2 weeks, once in about 3 weeks, once in about 4 weeks, once in about 2 months, once in about 3 months, once in about 4 months, once in about 5 months, or once in about 6 months.

50. The method of any one of embodiments 47 to 49, wherein the therapeutically effective dose of the vector is administered every 2 months and bevacizumab is administered every 2 weeks.

51. The method of any one of embodiments 1 to 50, further comprising administering to the subject an effective amount of one or more chemotherapeutic agents.

52. The method of embodiment 51, wherein the one or more chemotherapeutic agents are administered prior to, concurrently with, or after the administration of the priming dose of the vector.

53. The method of embodiment 52, wherein the one or more chemotherapeutic agents are administered prior to, concurrently with, or after the administration of the therapeutically effective dose of the vector.

54. The method of any one of embodiments 51 to 53, wherein the one or more chemotherapeutic agents are selected from the group consisting of altretamine, raltitrexed, topotecan, paclitaxel, docetaxel, cisplatin, carboplatin, oxaliplatin, liposomal doxorubicin, gemcitabine, cyclophosphamide, vinorelbine, ifosfamide, etoposide, altretamine, capecitabine, irinotecan, melphalan, pemetrexed, bevacizumab, and albumin bound paclitaxel.

55. The method of any one of embodiments 51 to 54, wherein the chemotherapeutic agent is paclitaxel.

56. The method of embodiment 55, wherein the paclitaxel is repeatedly administered.

57. The method of embodiment 56, wherein the paclitaxel is repeatedly administered every day, every two days, every three days, every four days, every five days, every six days, every seven days, every eight days, every nine days, or every ten days.

58. The method of any one of embodiments 55 to 57, wherein the therapeutically effective dose of the vector is administered every 2 months and paclitaxel is administered every week.

59. The method of any one of embodiments 1 to 58, wherein the subject exhibits a fever.

60. The method of embodiment 59, wherein the subject exhibits a fever after the administration of the priming dose.

61. A method of treating a tumor in a subject who is capable of exhibiting a febrile body temperature after administration of at least one priming dose of a vector which comprises a Fas-chimera gene operably linked to an endothelial cell-specific promoter, the method comprising administering a therapeutically effective dose of the vector to the subject after the subject exhibits a febrile body temperature after the administration of the priming dose.

62. A method of treating a tumor in a subject in need thereof comprising administering to the subject at least one priming dose of a vector which comprises a Fas-chimera gene operably linked to an endothelial cell-specific promoter and administering to the subject a therapeutically effective dose of the vector, wherein the subject has a febrile body temperature after the administration of the priming dose.

63. A method of treating a tumor in a subject in need thereof, the method comprising: (a) administering to the subject at least one priming dose of a vector which comprises a Fas-chimera gene operably linked to an endothelial cell-specific promoter; (b) determining the subject as having a febrile body temperature as a result of the administration of the priming dose in (a); and (c) administering a therapeutically effective dose of the vector to the subject having a febrile body temperature in (b).

64. A method of treating a tumor in a subject in need thereof, the method comprising: (a) administering to the subject at least one priming dose of a vector which comprises a Fas-chimera gene operably linked to an endothelial cell-specific promoter; (b) measuring the body temperature of the subject after the administration of the priming dose; (c) determining the subject as having a febrile body temperature as a result of the administration of the priming dose in (a); and (d) administering a therapeutically effective dose of the vector to the subject having a febrile body temperature in (c).

65. A method for identifying a responder to a Fas-chimera gene therapy, the method comprising administering to a subject having a tumor at least one priming dose of a vector which comprises a Fas-chimera gene operably linked to an endothelial cell-specific promoter, wherein the subject exhibits a febrile body temperature after the administration of the priming dose.

66. A method for identifying a responder to a Fas-chimera gene therapy, the method comprising administering to a subject having a tumor at least one priming dose of a vector which comprises a Fas-chimera gene operably linked to an endothelial cell-specific promoter and administering to the subject a therapeutically effective dose of the vector, wherein the subject exhibits a febrile body temperature after the administration of the priming dose.

67. A method for identifying a responder to a Fas-chimera gene therapy, the method comprising: (a) administering to a subject at least one priming dose of a vector which comprises a Fas-chimera gene operably linked to an endothelial cell-specific promoter; (b) identifying the subject as having a febrile body temperature as a result of the administration of the priming dose in (a); and (c) administering a therapeutically effective dose of the vector to the subject having a febrile body temperature in (b).

68. The method of any one of embodiments 61 to 67 wherein the priming dose is identical to the therapeutically effective dose.

69. The method of any one of embodiments 61 to 67, wherein the priming dose is lower than the therapeutically effective dose

70. The method of any one of embodiments 61 to 67, wherein the priming dose is higher than the therapeutically effective dose.

71. The method of any one of embodiments 61 to 67, wherein the priming dose is administered more than once.

### BRIEF DESCRIPTION OF THE DRAWINGS/FIGURES

FIG. 1 shows a schematic example of a study design in which patients received a regimen of an adenovirus comprising a FAS-chimera gene operably linked to an endothelial cell-specific promoter (e.g., VB-111) as a monotherapy or as part of a combination therapy with bevacizumab (BEV).

FIG. 2A-2C show patient tumor growth. FIG. 2A shows treatment through progression (TThP) in patients in monotherapy phase (n=24). FIG. 2B shows treatment through progression (TThP) in patients in the combination with bevacizumab phase (n=24). FIG. 2C shows tumor growth in patients in the limited exposure (LE) phase (n=22).

FIG. 3A-3D show survival curves for the treatment cohorts. FIG. 3A shows median progression-free survival (PFS) for patients in the Limited Exposure (LE) therapeutic dose group and the Treatment Through Progression (TThP) therapeutic dose group. FIG. 3B shows overall survival in the LE therapeutic dose group and the TThP therapeutic dose group. FIG. 3C shows overall survival in the TThP therapeutic dose group compared to historical overall survival for patients receiving AVASTIN® (bevacizumab) monotherapy. FIG. 3D shows the overall survival curve in patients exhibiting febrile response to VB-111 compared with patients who did not exhibit a febrile response to VB-111.

FIGS. 4A and 4B show VB-111 virus DNA levels in the blood of subjects after administration of VB-111. FIG. 4A shows viral DNA levels in a patient retaining basal levels (1×10¹³ vDNA copies/μg DNA) between doses, in the first 4 doses. FIG. 4B shows viral DNA levels in a subject dropping to zero between VB-111 doses.

FIG. 5 shows a correlation plot for overall survival (OS) vs. 27 cytokines for subjects in both the Continuous treatment group and the Limited treatment group.

FIG. 6 shows a correlation plot matrix for the data set comprising both the Continuous treatment group and the Limited treatment group.

FIG. 7 shows the overall survival (OS) for the Continuous treatment group and the Limited treatment group.

FIG. 8A shows the correlation plot for overall survival (OS) vs. 27 cytokines for subjects in the Limited treatment group. FIG. 8B shows the correlation plot matrix for the Limited treatment group.

FIG. 9A shows the correlation plot for overall survival (OS) vs. 27 cytokines for subjects in the Continuous treatment group. FIG. 9B shows the correlation plot matrix for the Continuous treatment group.

FIG. 10 shows a summary of the overall correlation data for subjects in both Continuous and Limited treatment groups.

FIG. 11 shows a summary of the correlation data for subjects in the Limited treatment group.

FIG. 12 shows a summary of the correlation data for subjects in the Continuous treatment group.

FIG. 13 shows the predicted regression line (Cox regression) with patient index level and overall survival day for Overall Data Set (both treatment groups).

FIG. 14 shows the predicted regression line (Cox regression) with patient index level and overall survival day for subjects in the Limited treatment group Data Set.

FIG. 15 shows the predicted regression line (Cox regression) with patient index level and overall survival day for subjects in the Continuous treatment group Data Set.

FIGS. 16A and 16B show the microwell plate layout for the Luminex mouse cytokine profiling assay. FIG. 16A shows the layout for the cytokine assay using anti-CD3/anti-CD28 for stimulation. FIG. 16B shows the layout for the cytokine assay using LPS for stimulation.

FIG. 17A-17G show the levels of cytokines from mouse spleen and microglial cells co-cultured with anti-CD3/anti-CD28. The following cytokines were measured: IL-1 (FIG. 17A); IP-10 (FIG. 17B); MCP-1 (FIG. 17C); MIP-2 (FIG. 17D); MIG (FIG. 17E); RANTES (FIG. 17F); and MIP-1β (FIG. 17G).

FIG. 18A-18N show the levels of cytokines from mouse spleen and microglial cells co-cultured with LPS. The following cytokines were measured: G-CSF (FIG. 18A); IFN-γ (FIG. 18B); IL-1α (FIG. 18C); IL-1β (FIG. 18D); IL-6 (FIG. 18E); IL-10 (FIG. 18F); IL-12 (FIG. 18G); IL-13 (FIG. 18H); MIP-1α (FIG. 18I); MIP-1β (FIG. 18J); RANTES (FIG. 18K); IL-9 (FIG. 18L); IL-15 (FIG. 18M); and M-CSF (FIG. 18N).

FIG. 19A-19E show the levels of cytokines from mouse spleen and tumor infiltrating T-cells co-cultured with anti-CD2/anti-CD28. The following cytokines were measured: IL-4 (FIG. 19A); IL-3 (FIG. 19B); IL-5 (FIG. 19C); IL-10 (FIG. 19D); and IL-13 (FIG. 19E).

FIG. 20A shows a comparison of IL-2 levels measured in co-cultures of splenocytes and microglial cells (“S+M”) or splenocytes and tumor-infiltrating T-cells (“S+T”) when stimulated with anti-CD3/anti-CD28. FIG. 20B shows a comparison of TNFα levels measured in co-cultures of splenocytes and microglial cells (“S+M”) or splenocytes and tumor-infiltrating T-cells (“S+T”) when stimulated with anti-CD3/anti-CD28.

FIG. 21A shows a comparison of VEGF levels measured in cultures of splenocytes (“S”), co-cultures of splenocytes and microglial cells (“S+T”), and co-cultures of splenocytes and tumor infiltrating T-cells (“S+T”) when stimulated with anti-CD3/anti-CD28. FIG. 21B shows a comparison of LIF levels measured in cultures of splenocytes (“S”), co-cultures of splenocytes and microglial cells (“S+T”), and co-cultures of splenocytes and tumor infiltrating T-cells (“S+T”) when stimulated with anti-CD3/anti-CD28.

FIG. 22 shows a comparison of LIF levels measured in cultures of splenocytes (“S”), co-cultures of splenocytes and microglial cells (“S+T”), and co-cultures of splenocytes and tumor infiltrating T-cells (“S+T”) when stimulated with LPS.

FIG. 23A-23E show correlation plots for cytokines in animals treated with Ad5-PPE-1-3X-Fas-c or control. FIG. 23A shows the correlation plot for Ad5-PPE-1-3X-Fas-c-treated animals, measuring cytokine profiles in a co-culture of splenocytes and microglia stimulated with anti-CD3/anti-CD28. FIG. 23B shows the correlation plot for control animals, measuring cytokine profiles in a co-culture of splenocytes and microglia stimulated with anti-CD3/anti-CD28. FIG. 23C shows the correlation plot for both treatment groups, measuring cytokine profiles in a co-culture of splenocytes and microglia stimulated with anti-CD3/anti-CD28. FIG. 23D shows another way of demonstrating the correlation in 3 cluster groups. FIG. 23E provides a summary of the data depicted in FIG. 23A-23D, showing the most significant cytokines from the experiment.

FIG. 24A-24C show correlation plots for cytokines in animals treated with Ad5-PPE-1-3X-Fas-c or control. FIG. 24A shows the correlation plot for Ad5-PPE-1-3X-Fas-c-treated animals, measuring cytokine profiles in a co-culture of splenocytes and microglia stimulated with LPS. FIG. 24B shows the correlation plot for control animals, measuring cytokine profiles in a co-culture of splenocytes and microglia stimulated with LPS. FIG. 24C shows the correlation plot for both treatment groups, measuring cytokine profiles in a co-culture of splenocytes and microglia stimulated with anti-CD3/anti-CD28. FIG. 24D shows the cytokines that had the greatest changes in profiles.

FIG. 25A-25B show the correlation between overall survival and subjects exhibiting fever. FIG. 25A shows the overall survival (OS) in subjects who exhibited a fever after the priming dose of vector (“yes”) compared with subjects who did not exhibit a fever after the priming dose of the vector (“no”). FIG. 25B shows the overall survival (OS) in subjects who exhibited a fever after any dose of vector (“yes”) compared with subjects who did not exhibit a fever after any dose of the vector (“no”).

FIG. 26 shows correlations between overall survival (OS) and subjects exhibiting a fever at any dose within each treatment group (Continuous or Limited).

FIG. 27A-27C show correlations between overall survival and cytokines associated with fever. The following cytokines were measured: IL-1α (FIG. 27A); IL-1β (FIG. 27B); and IL-6 (FIG. 27C).

FIG. 28A-28F show additional correlations between overall survival and cytokines associated with fever. The following cytokines were measured: IL-8 (FIG. 28A); TNFα (FIG. 28B); MIP-1α (FIG. 28C); MIP-1β (FIG. 28D); IFN-γ (FIG. 28E); and IFN-α (FIG. 28F).

FIG. 29 shows Kaplan Meier curves compiled from meta-analysis of eight studies of rGBM patients treated with bevacizumab monotherapy.

FIG. 30 shows Kaplan Meier curves compiled from meta-analysis of eight bevacizumab monotherapy studies (pooled as a single cohort) compared with data from the TThP cohort of the VB-111 study.

FIG. 31 shows Kaplan Meier curves compiled from meta-analysis of seven bevacizumab monotherapy studies (pooled as a single cohort) compared with data from the TThP cohort of the VB-111 study.

FIG. 32A-32C show immunohistochemistry staining of patient tumor biopsies from a patient treated with Ad5-PPE-1-3X-Fas-c every two months and paclitaxel every week. FIG. 32A shows baseline H&E staining (left) and CD8⁺ staining (right) before beginning treatment. FIG. 32B shows H&E staining (left) and CD8⁺ staining (right) one month after beginning treatment. FIG. 32C shows H&E staining (left) and CD8⁺ staining (right) 4.5 months after beginning treatment.

FIG. 33A-33H show immunohistochemistry staining of patient tumor biopsies from two patients (Pt 1 and Pt 2) treated with Ad5-PPE-1-3X-Fas-c every two months and paclitaxel every week. FIGS. 33A and 33B show CD8 staining and H&E staining (respectively) for patient 1 after treatment. FIGS. 33C and 33D show CD8 staining and H&E staining (respectively) for patient 2 after treatment. FIGS. 33E and 33F show CD8 staining and H&E staining (respectively) for patient 1 before treatment. FIGS. 33G and 33H show CD8 staining and H&E staining (respectively) for patient 2 before treatment. Circles and arrows indicate regions of apoptotic tumor cells.

### DETAILED DESCRIPTION OF THE DISCLOSURE

### I. Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order to further define this disclosure, the following terms and definitions are provided.

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

As used herein, “antibody” means an intact immunoglobulin, an antigen-binding fragment thereof, or an antigen-binding molecule. Antibodies of this disclosure can be of any isotype or class (e.g., M, D, G, E and A) or any subclass (e.g., G1-4, A1-2) and can have either a kappa (κ) or lambda (λ) light chain.

The term “effective amount” as used herein refers to an amount effective, at dosages and for periods of time necessary, to achieve a desired result. A desired result can be, for example, reduction or inhibition of neo-vascularization or angiogenesis in vitro or in vivo. An effective amount need not be a complete removal of neo-vascularization or angiogenesis.

As used herein, a “priming dose” refers to a dose of an agent administered to a subject prior to assessment of the subject's responsiveness to a therapy. A subject's responsiveness to a therapy can be assessed by MRI scan, CT scan, measurement of body temperature, measurement of a plasma biomarker or cell surface biomarker for angiogenesis or fever, and combinations thereof. After assessment of a subject's responsiveness to a therapy, a subsequent dose or subsequent doses of the same agent can be administered. The subsequent dose or subsequent doses are therapeutically effective doses of the agent. A priming dose can be a dosage amount lower than a therapeutically effective dose, a dosage amount identical to a therapeutically effective dose, or a dosage amount higher than a therapeutically effective dose.

As used herein, a “therapeutically effective dose” refers to a dose of an agent administered to a subject after an assessment of the subject's responsiveness to a therapy. A subject's responsiveness to a therapy can be assessed by MRI scan, CT scan, measurement of body temperature, measurement of a plasma biomarker or cell surface biomarker for fever, and combinations thereof. A therapeutically effective dose is an amount effective, at dosages and for periods of time necessary, to achieve a desired therapeutic result. A desired therapeutic result can be, e.g., lessening of symptoms, regression or stabilization of tumor size in radiological imaging, prolonged survival, improved mobility, and the like. A therapeutic result need not be a “cure.” In some embodiments, a therapeutically effective amount is an amount or dosage that is necessary to prevent occurrence of a tumor. In other aspects, a therapeutically effective amount is an amount or dosage that is necessary to reduce the size of a tumor. In other aspects, a therapeutically effective amount is an amount or dosage that is necessary to prevent recurrence of a tumor.

As used herein, a “prophylactically effective amount” refers to an amount effective, at dosages and for periods of time necessary, to achieve the desired prophylactic result. In some embodiments, since a prophylactic dose is used in subjects prior to or at an earlier stage of disease, the prophylactically effective amount will be less than the therapeutically effective amount.

As used herein, the phrase “treating a tumor” refers to inhibiting the growth of a tumor, reducing the size of a tumor, preventing the recurrence of a tumor, and combinations thereof.

The term “polynucleotide” or “nucleotide” is intended to encompass a singular nucleic acid as well as plural nucleic acids, and refers to an isolated nucleic acid molecule or construct, e.g., messenger RNA (mRNA) or plasmid DNA (pDNA). In certain embodiments, a polynucleotide comprises a conventional phosphodiester bond or a non-conventional bond (e.g., an amide bond, such as found in peptide nucleic acids (PNA)).

As used herein, a “polynucleotide,” “nucleotide,” or “nucleic acid” can be used interchangeably and contain the nucleotide sequence of the full-length cDNA sequence, including the untranslated 5′ and 3′ sequences, the coding sequences, as well as fragments, epitopes, domains, and variants of the nucleic acid sequence. The polynucleotide can be composed of any polyribonucleotide or polydeoxyribonucleotide, which can be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, the polynucleotides can be composed of triple-stranded regions comprising RNA or DNA or both RNA and DNA. Polynucleotides can also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

As used herein, “febrile body temperature,” “fever,” “fever symptoms”, and “pyrexia” can be used interchangeably and are intended to encompass a body temperature greater than what is considered normal body temperature in a subject. In a human, a normal body temperature is generally considered about 37° C. (about 98.6° F.), though this temperature can vary depending on factors such as the time of day the temperature is measured, route of temperature measurement (e.g., oral or rectal measurement), and intersubject variability. In some subjects—e.g., a human female subject—the normal body temperature can be higher than about 37° C. (about 98.6° F.). In other subjects—e.g., a human male subject—the normal body temperature can be lower than about 37° C. (about 98.6° F.). In a human, a febrile body temperature can comprise temperatures greater than about 37° C. (about 98.6° F.), greater than about 37.2° C. (about 99° F.), greater than about 37.5° C. (about 99.5° F.), greater than about 37.8° C. (about 100° F.), greater than about 38.0° C. (about 100.5° F.), greater than about 38.3° C. (about 100.9° F.), greater than about 38.5° C. (about 101.3° F.), greater than about 38.7° C. (about 101.7° F.), greater than about 38.9° C. (about 102° F.), greater than about 39° C. (about 102.2° F.), greater than about 39.2° C. (about 102.6° F.), greater than about 39.4° C. (about 102.9° F.), greater than about 39.6° C. (about 103.3° F.), greater than about 39.8° C. (about 103.6° F.), or greater than about 40° C. (about 104° F.).

In the present disclosure, a polypeptide can be composed of amino acids joined to each other by peptide bonds or modified peptide bonds, i.e., peptide isosteres, and can contain amino acids other than the 20 gene-encoded amino acids (e.g. non-naturally occurring amino acids). The polypeptides of the present disclosure can be modified by either natural process, such as posttranslational processing, or by chemical modification techniques which are well known in the art. Such modifications are well described in basic texts and in more detailed monographs, as well as in a voluminous research literature. Modifications can occur anywhere in the polypeptide, including the peptide backbone, the amino acid side-chains and the amino or carboxyl termini. It will be appreciated that the same type of modification can be present in the same or varying degrees at several sites in a given polypeptide. Also, a given polypeptide can contain many types of modifications. Polypeptides can be branched, for example, as a result of ubiquitination, and they can be cyclic, with or without branching. Cyclic, branched, and branched cyclic polypeptides can result from posttranslation natural processes or can be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins—Structure And Molecular Properties, 2nd Ed., T. E. Creighton, W.H. Freeman and Company, New York (1993); Posttranslational Covalent Modification of Proteins, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992).)

The terms “fragment,” “variant,” “derivative” and “analog” when referring to any polypeptide or polynucleotide of the present disclosure include any polypeptides or polynucleotides which retain at least some activities, i.e., the ability to function as any naturally-occurring function of the polypeptide or polynucleotide. For example, a “fragment,” “variant,” “derivative” and “analog” of Tumor necrosis factor Receptor 1 (TNFR1) has some activities of the naturally occurring full-length TNFR1, e.g., the ability to bind to TNFR1 ligand, i.e., TNF-alpha or lymphotoxin. In another example, a “fragment,” “variant,” “derivative” and “analog” of a FAS polypeptide have some activities of a naturally-occurring full-length FAS polypeptide, e.g., the ability to induce apoptosis. In other examples, a “fragment,” “variant,” “derivative” and “analog” of an endothelial cell-specific promoter can induce endothelial cell-specific expression of a gene operably linked to the promoter. Additional non-limiting examples of the various fragments, variants, analogues, or derivatives of the TNFR1, FAS polypeptide, and endothelial cell-specific promoters are described below.

In the present disclosure, a “polypeptide fragment” or “protein fragment” refers to a short amino acid sequence of a polypeptide. Protein or polypeptide fragments can be “free-standing,” or comprised within a larger polypeptide of which the fragment forms a part of region. Representative examples of polypeptide fragments of the disclosure, include, for example, fragments comprising about 5 amino acids, about 10 amino acids, about 15 amino acids, about 20 amino acids, about 30 amino acids, about 40 amino acids, about 50 amino acids, about 60 amino acids, about 70 amino acids, about 80 amino acids, about 90 amino acids, or about 100 amino acids.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another embodiment, a string of amino acids can be conservatively replaced with a structurally similar string that differs in order and/or composition of side chain family members.

The term “percent sequence identity” between two polynucleotide or polypeptide sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence.

The percentage of sequence identity is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences can be accomplished using readily available software both for online use and for download. Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

One skilled in the art will appreciate that the generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is ClustalW2, available from www.clustal.org. Another suitable program is MUSCLE, available from www.drive5.com/muscle/. ClustalW2 and MUSCLE are alternatively available, e.g., from the EBI.

It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

As used herein, the terms “linked,” “fused,” “fusion,” “chimeric,” and “chimera” are used interchangeably. These terms refer to the joining together of two more elements or components, by whatever means including chemical conjugation or recombinant means. An “in-frame fusion” refers to the joining of two or more open reading frames (ORFs) to form a continuous longer ORF, in a manner that maintains the correct reading frame of the original ORFs. Thus, the resulting recombinant fusion or chimeric protein is a single protein containing two or more segments that correspond to polypeptides encoded by the original ORFs (which segments are not normally so joined in nature.) Although the reading frame is thus made continuous throughout the fused segments, the segments can be physically or spatially separated by, for example, in-frame linker sequence.

The terms “heterologous” and “heterologous moiety” mean that a polynucleotide, polypeptide, or other moiety is derived from a distinct entity from that of the entity to which it is being compared. For instance, a heterologous polypeptide can be synthetic, or derived from a different species, different cell type of an individual, or the same or different type of cell of distinct individuals. In one aspect, a heterologous moiety can be a polypeptide fused to another polypeptide to produce a fusion polypeptide or protein. In another aspect, a heterologous moiety can be a non-polypeptide such as PEG conjugated to a polypeptide or protein.

In the context of polypeptides, a “linear sequence” or a “sequence” is an order of amino acids in a polypeptide in an amino to carboxyl terminal direction in which residues that neighbor each other in the sequence are contiguous in the primary structure of the polypeptide.

The term “expression” as used herein refers to a process by which a gene produces a biochemical, for example, an RNA or polypeptide. The process includes any manifestation of the functional presence of the gene within the cell including, without limitation, gene knockdown as well as both transient expression and stable expression. It includes without limitation transcription of the gene into messenger RNA (mRNA), transfer RNA (tRNA), small hairpin RNA (shRNA), small interfering RNA (siRNA) or any other RNA product and the translation of such mRNA into polypeptide(s). If the final desired product is biochemical, expression includes the creation of that biochemical and any precursors.

The term “complementarity determining region” (CDR) as used herein refers to the amino acid residues of an antibody which are responsible for binding to an antigen. The CDR regions of an antibody are found within the hypervariable region of both heavy and light chains of the antibody. Full length antibodies comprise three CDR regions in the heavy chain variable domain and three CDR regions in the light chain variable domain.

The term “repeatedly administered” as used herein refers to administration of a therapeutic agent on a repeated basis at defined, fixed intervals. The intervals of time between each administration can be altered during the course of the repeated administration and can be as long as 1 day, 2 days, 3 days, 4 days, 5 days, 6 days, 7 days, 2 weeks, 3 weeks, 4 weeks, 5 weeks, 6 weeks, 7 weeks, 2 months, 3 months, 4 months, 5 months, 6 months, 7 months, 8 months, 9 months, 10 months, 11 months, 1 year, or more.

The term “combination therapy” as used herein refers to the administration of two or more therapeutic modalities to treat a disease or condition. In one aspect of the present disclosure, combination therapy refers to the administration of a vector and a VEGF antagonist to a subject or a subject population in need thereof. In some embodiments, the combination therapy comprises administering the VEGF antagonist prior to administering the vector. In another embodiment, the combination therapy comprises administering the VEGF antagonist concomitantly with administration of the vector. In another embodiment, the combination therapy comprises administering the VEGF antagonist after administering the vector.

### II. Nucleic Acid Constructs Comprising a Fas-Chimera Gene and an Endothelial Cell Specific Promoter

The present disclosure is related to methods of treating a tumor in a subject who is capable of exhibiting a febrile body temperature after administration of at least one priming dose of a vector which comprises a Fas-chimera gene operably linked to an endothelial cell-specific promoter, the method comprising administering a therapeutically effective dose of the vector to the subject after the subject exhibits a febrile body temperature after the administration of the priming dose. The gene encoding the FAS-chimera protein (or gene product), in the present disclosure can be linked to an endothelial cell-specific promoter, which directs expression of the FAS-chimera gene product in an endothelial cell. Expression of such a cytotoxic gene product is useful in a situation where excessive neo-vascularization or blood vessel growth is not desirable, e.g., in a tumor.

The present disclosure also provides a method for identifying a responder to a Fas-chimera gene therapy, the method comprising administering to a subject having a tumor at least one priming dose of a vector which comprises a Fas-chimera gene operably linked to an endothelial cell-specific promoter, wherein the subject exhibits a febrile body temperature after the administration of the priming dose.

A. FAS-Chimera

A FAS-chimera protein expressed by the nucleic acid construct of the disclosure comprises at least two “death receptor” polypeptides, each of the polypeptides is derived from a different protein. The first polypeptide of the FAS-chimera protein comprises a ligand binding domain of Tumor Necrosis Factor Receptor 1 (TNFR1). The second polypeptide of the FAS-chimera protein comprises an effector domain of a FAS polypeptide.

The ligand binding domain of TNFR1 can be any domain that binds to a TNFR1 ligand. In one embodiment, the TNFR1 ligand is TNF-α. In another embodiment, the TNFR1 ligand is lymphotoxin-a. The ligand binding domain of TNFR1 can be an extracellular domain of TNFR1 or any fragments, variants, derivatives, or analogues thereof. Non-limiting examples of the TNFR1 ligand binding domain are described below.

The effector domain of a FAS polypeptide useful for the disclosure comprises any FAS domains that form death-inducing signaling complex (DISC), thereby inducing apoptosis. In one embodiment, an effector domain of a FAS polypeptide comprises an intracellular domain, a trans-membrane domain, or both. Non-limiting examples of FAS polypeptide effector domains are described below.

The TNFR1 and the FAS polypeptide can be linked by a peptide bond or by a linker. The linker connecting the TNFR1 ligand binding domain with the FAS effector domain can be a polypeptide linker or a non-peptide linker. For example, a linker for the FAS-chimera protein can comprise one or more glycine, serine, leucine, or any combinations thereof. In one embodiment, a linker useful for the disclosure comprises Ser-Leu. In another embodiment, a linker useful for the disclosure comprises (GGGS)n, (Denise et al. J. Biol. Chem. 277:35035-35043 (2002)), wherein n can be 0, 1, 2, 3, 4, 5, 6, 7, 8, 9, or 10 or more (SEQ ID NO: 27).

1. Tumor Necrosis Factor Receptor 1

The full-length human TNFR1 polypeptide is 455 amino acids in length and is also known as TNF-R1, Tumor necrosis factor receptor type I (TNFRI), TNFR-I, TNFRSF1A, TNFAR, p55, P60, or CD120a. Naturally-occurring human TNFR1 polypeptide is known to bind to TNF-α or homotrimeric lymphotoxin-α. Binding of TNF-α to the extracellular domain leads to homotrimerization of TNFR1, which then interacts specifically with the death domain of Tumor Necrosis Factor Receptor Type 1-Associated Death Domain Protein (TRADD). Various TRADD-interacting proteins such as TNF Receptor Associated Factors (TRAFS), Receptor-Interacting Serine/Threonine-Protein Kinase 1 (RIPK1), and Fas-Associated Protein with Death Domain (FADD) are recruited to the complex by their association with TRADD. The complex activates at least two distinct signaling cascades, apoptosis and NF-kappa-B signaling.

A 455 aa polypeptide sequence reported as a human TNFR1 polypeptide sequence has the identifier number P19438-1 in the UniProtKB database. This human TNFR1 polypeptide sequence is designated herein as isoform A and SEQ ID NO: 2. SEQ ID NO: 1 is a nucleotide sequence encoding SEQ ID NO: 2. A polypeptide sequence of 108 aa was reported as an isoform of the human TNFR1 polypeptide sequence and has the identifier number P19438-2 in the UniProtKB database. The 108 aa polypeptide corresponds to amino acids 1 to 108 of isoform A (SEQ ID NO: 2) and is designated herein as isoform B. Another variant of the human TNFR1 polypeptide having 232 aa was reported as the identifier number P19438-3 in the UniProtKB database. The 232 aa polypeptide corresponds to amino acids 1 to 232 of isoform A (SEQ ID NO: 2) and is designated herein as isoform C. Additional natural variants of human TNFR1 include, but are not limited to, the TNFR1 polypeptide of isoforms A, B, and C comprising one or more mutations selected from the group consisting of H51Q, C59R, C59S, C62G, C62Y, P75L, T79M, C81F, C99S, S115G, C117R, C117Y, R121P, R121Q, P305T, and any combinations thereof. Other known TNFR1 variants include the TNFR1 polypeptide of isoforms A, B, and C comprising L13LILPQ, K255E, S286G, R394L, 412:Missing, GPAA443-446APP, or any combinations thereof.

Table 1 shows the human wild-type TNFR1 amino acid sequence and a nucleotide sequence encoding the wild-type TNFR1.

TABLE 1
TNFR1 Sequences

| SEQ ID | |
|---|---|
| No. | Sequences |
| Amino | MGLSTVPDLLLPLVLLELLVGIYPSGVIGLVPHLGDREKRDSVCPQGKYIHPQNNSICCT |
| acid | KCHKGTYLYNDCPGPGQDTDCRECESGSFTASENHLRHCLSCSKCRKEMGQVEISSCTVD |
| sequence | RDTVCGCRKNQYRHYWSENLFQCFNCSLCLNGTVHLSCQEKQNTVCTCHAGFFLRENECV |
| of TNFR1 | SCSNCKKSLECTKLCLPQIENVKGTEDSGTTVLLPLVIFFGLCLLSLLFIGLMYRYQRWK |
| (SEQ ID | SKLYSIVCGKSTPEKEGELEGTTTKPLAPNPSFSPTPGETPTLGFSPVPSSTFTSSSTYT |
| NO: 2) | PGDCPNFAAPRREVAPPYQGADPILATALASDPIPNPLQKWEDSAHKPQSLDTDDPATLY |
| | AVVENVPPLRWKEFVRRLGLSDHEIDRLELQNGRCLREAQYSMLATWRRRTPRREATLEL |
| | LGRVLRDMDLLGCLEDIEEALCGPAALPPAPSLLR |
| Nucleotide | atgggcctctccaccgtgcctgacctgctgctgccgctggtgctcctggagctgttggtg |
| Sequence | aaaatatacccctcaggggttattggactggtccctcacctaggggacagggagaagaga |
| encoding | gatagtgtgtgtccccaaggaaaatatatccaccctcaaaataattcgatttgctgtacc |
| TNFR1 | aagtgccacaaaggaacctacttgtacaatgactgtccaggcccggggcaggatacggac |
| (SEQ ID | tgcagggagtgtgagagcggctccttcaccgcttcagaaaaccacctcagacactgcctc |
| NO: 1) | agctgctccaaatgccgaaaggaaatgggtcaggtggagatctcttcttgcacagtggac |
| | cgggacaccgtgtgtggctgcaggaagaaccagtaccggcattattggagtgaaaacctt |
| | ttccagtgcttcaattgcagcctctgcctcaatgggaccgtgcacctctcctgccaggag |
| | aaacagaacaccgtgtgcacctgccatgcaggtttctttctaagagaaaacgagtgtgtc |
| | tcctgtagtaactgtaagaaaagcctggagtgcacgaagttgtgcctaccccagattgag |
| | aatgttaagggcactgaggactcaggcaccacagtgctgttgcccctggtcattttcttt |
| | ggtctttgccttttatccctcctcttcattggtttaatgtatcgctaccaacggtggaag |
| | tccaagctctactccattgtttgtgggaaatcgacacctgaaaaagagggggagcttgaa |
| | ggaactactactaagcccctggccccaaacccaagcttcagtcccactccaggcttcacc |
| | cccaccctgggcttcagtcccgtgcccagttccaccttcacctccagctccacctatacc |
| | cccggtgactgtcccaactttgcggctccccgcagagaggtggcaccaccctatcagggg |
| | gctgaccccatccttgcgacagccctcgcctccgaccccatccccaacccccttcagaag |
| | tgggaggacagcgcccacaagccacagagcctagacactgatgaccccgcgacgctgtac |
| | gccgtggtggagaacgtgcccccgttgcgctggaaggaattcgtgcggcgcctagggctg |
| | agcgaccacgagatcgatcggctggagctgcagaacgggcgctgcctgcgcgaggcgcaa |
| | tacagcatgctggcgacctggaggcggcgcacgccgcggcgcgaggccacgctggagctg |
| | ctgggacgcgtgctccgcgacatggacctgctgggctgcctggaggacatcgaggaggcg |
| | ctttgcggccccgccgccctcccgcccgcgcccagtcttctcaga |
| Amino | MGLSTVPDLLLPLVLLELLVGIYPSGVIGLVPHLGDREKRDSVCPQGKYIHPQNNSICCT |
| acid | KCHKGTYLYNDCPGPGQDTDCRECESGSFTASENHLRHCLSCSKCRKEMGQVEISSCTVD |
| sequence | RDTVCGCRKNQYRHYWSENLFQCFNCSLCLNGTVHLSCQEKQNTVCTCHAGFFLRENECV |
| of a | SCSNCKKSLECTKLCLP |
| Ligand | |
| Binding | |
| Domain of | |
| TNFR1 | |
| (SEQ ID | |
| NO: 4) | |
| Nucleotide | atgggcctct ccaccgtgcc tgacctgctg ctgccgctgg tgctcctgga |
| sequence | gctgttggtg ggaatatacc cctcaggggt tattggactg gtccctcacc |
| encoding a | taggggacag ggagaagaga gatagtgtgt gtccccaagg aaaatatatc |
| Ligand | caccctcaaa ataattcgat ttgctgtacc aagtgccaca aaggaaccta |
| Binding | cttgtacaat gactgtccag gcccggggca ggatacggac tgcagggagt |
| Domain of | gtgagagcgg ctccttcacc gcttcagaaa accacctcag acactgcctc |
| TNFR1 | agctgctcca aatgccgaaa ggaaatgggt caggtggaga tctcttcttg |
| (SEQ ID | cacagtggac cgggacaccg tgtgtggctg caggaagaac cagtaccggc |
| NO: 3) | attattggag tgaaaacctt ttccagtgct tcaattgcag cctctgcctc |
| | aatgggaccg tgcacctctc ctgccaggag aaacagaaca ccgtgtgcac |
| | ctgccatgca ggtttctttc taagagaaaa cgagtgtgtc tcctgtagta |
| | actgtaagaa aagcctggag tgcacgaagt tgtgcctacc a |

The mouse TNFR1 polypeptide sequence and its variants are also reported. The 454 aa mouse TNFR1 polypeptide has the identifier number P25118 in UniProtKB database. TNFR1 polypeptides known in other animals include, but are not limited to, rat (e.g., P22934 in the UniProtKB database), cow (e.g., 019131 in the UniProtKB database), pig (e.g., P50555 in the UniProtKB database), or horse (e.g., D1MH71 in the UniProtKB database).

The full-length TNFR1 can be cleaved into two chains, (1) TNF Receptor Superfamily Member 1A, membrane form (i.e., amino acids 22 to 455 corresponding to full-length TNFR1) and (2) TNF-binding protein 1 (TBPI) (i.e., amino acids 41 to 291 corresponding to full-length TNFR1). The full-length human TNFR1 polypeptide consists of a signal sequence (amino acids 1 to 21 of SEQ ID NO: 2), an extracellular domain (amino acids 22 to 211 of SEQ ID NO: 2), a trans-membrane domain (amino acids 212 to 234 of SEQ ID NO: 2), and a cytoplasmic domain (amino acids 235 to 455 of SEQ ID NO: 2). The TNFR1 extracellular domain comprises four cysteine repeat regions, TNFR-Cys1 (amino acids 43 to 82 corresponding to SEQ ID NO: 2), TNFR-Cys2 (amino acids 83 to 125 corresponding to SEQ ID NO: 2), TNFR-Cys3 (amino acids 126 to 166 corresponding to SEQ ID NO: 2), and TNFR-Cys4 (amino acids 167 to 196 corresponding to SEQ ID NO: 2).

As one of skill in the art will appreciate, the beginning and ending residues of the domains listed above can vary depending upon the computer modeling program used or the method used for determining the domain. As such, various functional domains of TNFR1 can vary from those defined above.

In one embodiment, a ligand binding domain of TNFR1 useful for the FAS-chimera protein comprises, consists essentially of, or consists of an extracellular domain of TNFR1, or any fragment, variant, derivative, or analogue thereof, wherein the extracellular domain of TNFR1, or any fragment, variant, derivative, or analogue thereof binds to TNF-α. In another embodiment, a ligand binding domain of TNFR1 comprises TNFR-Cys1; TNFR-Cys2; TNFR-Cys3; TNFR-Cys4; TNFR-Cys1 and TNFR-Cys2; TNFR-Cys1 and TNFR-Cys3; TNFR-Cys1 and TNFR-Cys4; TNFR-Cys2 and TNFR-Cys3; TNFR-Cys2 and TNFR-Cys4; TNFR-Cys3 and TNFR-Cys4; TNFR-Cys1, TNFR-Cys2, and TNFR-Cys3; TNFR-Cys1, TNFR-Cys2, and TNFR-Cys4; TNFR-Cys2, TNFR-Cys3, and TNFR-Cys4; or TNFR-Cys1, TNFR-Cys2, TNFR-Cys3, and TNFR-Cys4. In other embodiments, a ligand binding domain of TNFR1 in the FAS-chimera protein comprises TNF binding protein I. In yet other embodiments, a TNFR1 ligand binding domain of the FAS-chimera protein comprises, consists essentially of, or consists of an amino acid sequence at least 60%, 70%, 80%, 90%, 95%, 96%, 97%, 98%, 99%, or 100% identical to amino acids 22 to 190, amino acids 22 to 191, amino acids 22 to 192, amino acids 22 to 193, amino acids 22 to 194, amino acids 22 to 195, amino acids 22 to 196, amino acids 22 to 197, amino acids 22 to 198, amino acids 22 to 199, amino acids 22 to 200, amino acids 22 to 201, amino acids 22 to 202, amino acids 22 to 203, amino acids 22 to 204, amino acids 22 to 205, amino acids 22 to 206, amino acids 22 to 207, amino acids 22 to 208, amino acids 22 to 209, amino acids 22 to 210, or amino acids 22 to 211 of SEQ ID NO: 2, wherein the ligand binding domain binds to a TNFR1 ligand, e.g., TNF-α.

In other embodiments, the ligand binding domain of TNFR1 further comprises a signal peptide. One example of the suitable signal peptides is the signal peptide of TNFR1, e.g., amino acids 1 to 21 of SEQ ID NO: 2. In yet other embodiments, a ligand binding domain of the FAS-chimera gene product comprises, consists essentially of, or consists of an amino acid sequence at least 60%, 70%, 80%, 90%, 95%, 96%, 97%, 98%, 99%, or 100% identical to amino acids 1 to 190, amino acids 1 to 191, amino acids 1 to 192, amino acids 1 to 193, amino acids 1 to 194, amino acids 1 to 195, amino acids 1 to 196, amino acids 1 to 197, amino acids 1 to 198, amino acids 1 to 199, amino acids 1 to 200, amino acids 1 to 201, amino acids 1 to 202, amino acids 1 to 203, amino acids 1 to 204, amino acids 1 to 205, amino acids 1 to 206, amino acids 1 to 207, amino acids 1 to 208, amino acids 1 to 209, amino acids 1 to 210, or amino acids 1 to 211 of SEQ ID NO: 2, wherein the ligand binding domain binds to a TNFR1 ligand, e.g., TNF-α. In a specific embodiment, a TNFR1 ligand binding domain of the FAS-chimera protein comprises, consists essentially of, or consists of an amino acid sequence at least 60%, 70%, 80%, 90%, 95%, 96%, 97%, 98%, 99%, or 100% identical to SEQ ID NO: 4, wherein the ligand binding domain binds to a TNFR1 ligand, e.g., TNF-α.

In yet other embodiments, the ligand binding domain of TNFR1 is encoded by a nucleotide sequence at least 60%, 70%, 80%, 90%, 95%, 96%, 97%, 98%, 99%, or 100% identical to SEQ ID NO: 3.

In still other embodiments, a TNFR1 ligand binding domain of the FAS-chimera protein comprises, consists essentially of, or consists of an amino acid sequence at least 60%, 70%, 80%, 90%, 95%, 96%, 97%, 98%, 99%, or 100% identical to amino acids 22 to 108 of SEQ ID NO: 2 (TNFR1 isoform B), amino acids 22 to 232 of SEQ ID NO: 2 (TNFR1 isoform C), or amino acids 44 to 291 of SEQ ID NO: 2 (TBP1), wherein the ligand binding domain binds to a TNFR1 ligand, e.g., TNF-α.

2. FAS Polypeptide

The full-length human FAS polypeptide is 335 amino acids in length and is also known as Tumor Necrosis Factor Receptor Superfamily Member 6, Apo-1 antigen, Apoptosis-mediating surface antigen FAS, FASLG receptor, or CD95. Naturally occurring FAS polypeptide is a receptor for TNFSF6/FASLG. When the FAS polypeptide binds to the FAS ligand (FasL), the interaction between FAS and FasL results in the formation of the death-inducing signaling complex (DISC), which contains the FADD, caspase-8 and caspase-10. In some types of cells (type I), processed caspase-8 directly activates other members of the caspase family, and triggers the execution of apoptosis of the cell. In other types of cells (type II), the FAS-DISC starts a feedback loop that spirals into increasing release of proapoptotic factors from mitochondria and the amplified activation of caspase-8. FAS-mediated apoptosis can have a role in the induction of peripheral tolerance, in the antigen-stimulated suicide of mature cells or both.

A 335 aa polypeptide sequence reported as a human FAS polypeptide sequence has the identifier number P25445-1 in the UniProtKB database. This human FAS polypeptide sequence is designated herein as SEQ ID NO: 6. SEQ ID NO: 5 is a nucleotide sequence encoding SEQ ID NO: 6. The nucleotide sequence encoding the FAS polypeptide is also known as APT1, FAS1, or TNFRSF6. The full-length FAS polypeptide contains a signal peptide (amino acids 1 to 25 corresponding to SEQ ID NO: 6), an extracellular domain (amino acids 26 to 173 corresponding to SEQ ID NO: 6), a trans-membrane domain (amino acids 174 to 190 corresponding to SEQ ID NO: 6), and an intracellular (or cytoplasmic) domain (amino acids 191 to 335 corresponding to SEQ ID NO: 6). The intracellular domain contains a death domain (e.g., amino acids 230 to 314 corresponding to SEQ ID NO: 6).

As one of skill in the art will appreciate, the beginning and ending residues of the domains listed above can vary depending upon the computer modeling program used or the method used for determining the domain. As such, various functional domains of FAS can vary from those defined above. Table 2 shows the wild-type human FAS amino acid sequence and a nucleotide sequence encoding the FAS protein.

TABLE 2
FAS Sequences

| | Sequences |
|---|---|
| Amino acid | MLGIWTLLPLVLTSVARLSSKSVNAQVTDINSKGLELRKTVTTVETQNLEGLHHDGQFCH |
| sequence of | KPCPPGERKARDCTVNGDEPDCVPCQEGKEYTDKAHFSSKCRRCRLCDEGHGLEVEINCT |
| human FAS | RTQNTKCRCKPNFFCNSTVCEHCDPCTKCEHGIIKECTLTSNTKCKEEGSRSNLGWLCLL |
| protein | LLPIPLIVWVKRKEVQKTCRKHRKENQGSHESPTLNPETVAINLSDVDLSKYITTIAGVM |
| (SEQ ID | TLSQVKGFVRKNGVNEAKIDEIKNDNVQDTAEQKVQLLRNWHQLHGKKEAYDTLIKDLKK |
| NO: 6) | ANLCTLAEKIQTIILKDITSDSENSNFRNEIQSLV |
| Nucleotide | atgctgggcatctggaccctcctacctctggttcttacgtctgttgctagattatcgtcc |
| sequence | aaaagtgttaatgcccaagtgactgacatcaactccaagggattggaattgaggaagact |
| encoding | gttactacagttgagactcagaacttggaaggcctgcatcatgatggccaattctgccat |
| human FAS | aagccctgtcctccaggtgaaaggaaagctagggactgcacagtcaatggggatgaacca |
| sequence | gactgcgtgccctgccaagaagggaaggagtacacagacaaagcccatttttcttccaaa |
| (SEQ ID | tgcagaagatgtagattgtgtgatgaaggacatggcttagaagtggaaataaactgcacc |
| NO: 5) | cggacccagaataccaagtgcagatgtaaaccaaactttttttgtaactctactgtatgt |
| | gaacactgtgacccttgcaccaaatgtgaacatggaatcatcaaggaatgcacactcacc |
| | agcaacaccaagtgcaaagaggaaggatccagatctaacttggggtggctttgtcttctt |
| | cttttgccaattccactaattgtttgggtgaagagaaaggaagtacagaaaacatgcaga |
| | aagcacagaaaggaaaaccaaggttctcatgaatctccaactttaaatcctgaaacagtg |
| | gcaataaatttatctgatgttgacttgagtaaatatatcaccactattgctggagtcatg |
| | acactaagtcaagttaaaggctttgttcgaaagaatggtgtcaatgaagccaaaatagat |
| | gagatcaagaatgacaatgtccaagacacagcagaacagaaagttcaactgcttcgtaat |
| | tggcatcaacttcatggaaagaaagaagcgtatgacacattgattaaagatctcaaaaaa |
| | gccaatctttgtactcttgcagagaaaattcagactatcatcctcaaggacattactagt |
| | gactcagaaaattcaaacttcagaaatgaaatccaaagcttggtctag |
| Amino acid | GSRSNLGWLCLLLLPIPLIVWVKRKEVQKTCRKHRKENQGS |
| sequence of | HESPTLNPETVAINLSDVDLSKYITTIAGVMTLSQVKGFVR |
| an Effector | KNGVNEAKIDEIKNDNVQDTAEQKVQLLRNWHQLHGKKEAY |
| Domain of | DTLIKDLKKANLCTLAEKIQTIILKDITSDSENSNFRNEIQ |
| FAS (SEQ | SLV |
| ID NO: 8) | |
| Nucleotide | aggatccagatctaacttggggtggctttgtcttcttcttttgccaattccactaatt |
| sequence | gtttgggtgaagagaaaggaagtacagaaaacatgcagaaagcacagaaaggaaaacc |
| encoding an | aaggttctcatgaatctccaaccttaaatcctgaaacagtggcaataaatttatctga |
| Effector | tgttgacttgagtaaatatatcaccactattgctggagtcatgacactaagtcaagtt |
| Domain of | aaaggctttgttcgaaagaatggtgtcaatgaagccaaaatagatgagatcaagaatg |
| FAS (SEQ | acaatgtccaagacacagcagaacagaaagttcaactgcttcgtaattggcatcaact |
| ID NO: 7) | tcatggaaagaaagaagcgtatgacacattgattaaagatctcaaaaaagccaatctt |
| | tgtactcttgcagagaaaattcagactatcatcctcaaggacattactagtgactcag |
| | aaaattcaaacttcagaaatgaaatccaaagcttggtctag |

The mouse FAS polypeptide sequence and its variants are also reported. The 327 aa mouse FAS polypeptide has the identifier number P25446 in UniProtKB database. FAS polypeptides known in other animals include, but are not limited to, Old World monkey (e.g., Q9BDN4 in the UniProtKB database), Rhesus monkey (e.g., Q9BDP2 in the UniProtKB database), rat (e.g., Q63199 in the UniProtKB database), or cow (e.g., P51867 in the UniProtKB database).

Based on the sequence variation in the FAS polypeptide, a person of ordinary skill in the art can identify sequence variations in the effector domain of the FAS polypeptide. For example, natural variants of the FAS effector domains can include one or more substitutions or mutations of C178R, L180F, P183L, I184V, T1981, Y232C, T241K, T241P, V249L, R250P, R250Q, G253D, G253S, N255D, A257D, I259R, D260G, D260V, D260Y, I262S, N264K, T2701, T270K, E272G, E272K, L278F, K299N, T3051, I310S, or any combinations thereof.

In one embodiment, an effector domain of the FAS polypeptide useful for the disclosure comprises a death domain of the FAS polypeptide. In another embodiment, an effector domain of the FAS polypeptide comprises, consists essentially of, or consists of an amino acid sequence at least 60%, 70%, 80%, 90%, 95%, 96%, 97%, 98%, 99%, or 100% identical to amino acids 230 to 314 of SEQ ID NO: 6. In other embodiments, an effector domain of the FAS polypeptide comprises an intracellular domain of the FAS polypeptide. In yet other embodiments, an effector domain of the FAS polypeptide comprises an amino acid sequence at least 60%, 70%, 80%, 90%, 95%, 96%, 97%, 98%, 99%, or 100% identical to amino acids 185 to 335, amino acids 186 to 335, amino acids 187 to 335, amino acids 188 to 335, amino acids 189 to 335, amino acids 190 to 335, amino acids 191 to 335, amino acids 192 to 335, amino acids 193 to 335, amino acids 194 to 335, amino acids 195 to 335, amino acids 196 to 335, amino acids 197 to 335, amino acids 198 to 335, or amino acids 199 to 335 of SEQ ID NO: 6.

In still other embodiments, the effector domain of the FAS polypeptide further comprises a trans-membrane domain of the FAS polypeptide. In yet other embodiments, an effector domain of the FAS polypeptide comprises an amino acid sequence at least about 60%, 70%, 80%, 90%, 95%, 96%, 97%, 98%, 99%, or 100% identical to amino acids 174 to 335 of SEQ ID NO: 6. In some embodiments, an effector domain of the FAS polypeptide further comprises about ten, about nine, about eight, about seven, about six, about five, about four, about three, about two, or about one amino acid from the C-terminal portion of the FAS extracellular domain. In certain embodiments, an effector domain of the FAS polypeptide comprises an amino acid sequence at least 60%, 70%, 80%, 90%, 95%, 96%, 97%, 98%, 99%, or 100% identical to amino acids 179 to 335, amino acids 178 to 335, amino acids 177 to 335, amino acids 176 to 335, amino acids 175 to 335, amino acids 174 to 335, amino acids 173 to 335, amino acids 172 to 335, amino acids 171 to 335, amino acids 170 to 335, amino acids 169 to 335, amino acids 168 to 335, amino acids 167 to 335, amino acids 166 to 335, amino acids 165 to 335, amino acids 164 to 335, or amino acids 163 to 335 of SEQ ID NO: 6, wherein the effector domain forms a death-inducing signaling complex (DISC), activates caspase 8, or induces apoptosis.

In some embodiments, an effector domain of the FAS polypeptide comprises, consists essentially of, or consists of an amino acid sequence at least 70%, 80%, 90%, 95%, 96%, 97%, 98%, 99%, or 100% identical to SEQ ID NO: 8, wherein the effector domain forms a death-inducing signaling complex (DISC), activates caspase 8, or induces apoptosis.

In other embodiments, an effector domain of the FAS polypeptide is encoded by a nucleotide sequence at least 60%, 70%, 80%, 90%, 95%, 96%, 97%, 98%, 99%, or 100% identical to SEQ ID NO: 7.

In one embodiment, the FAS-chimera gene product for the disclosure comprises, consists essentially of, or consists of an amino acid sequence at least 70%, 80%, 90%, 95%, 96%, 97%, 98%, 99%, or 100% identical to SEQ ID NO: 10, wherein the FAS-chimera gene product induces apoptosis. In another embodiment, the FAS-chimera gene product is encoded by a nucleotide sequence at least 60%, 70%, 80%, 90%, 95%, 96%, 97%, 98%, 99%, or 100% identical to SEQ ID NO: 9, wherein the FAS-chimera gene product induces apoptosis.

B. Endothelial Cell-Specific Promoter

The nucleic acid construct comprising a FAS-chimera gene further comprises one or more expression control elements useful for regulating the expression of an operably linked FAS-chimera gene. The expression control elements include, but are not limited to, promoters, secretion signals, and other regulatory elements.

The nucleic acid construct useful for the present disclosure utilizes an endothelial cell-specific promoter to direct expression of the FAS-chimera protein in an endothelial cell, thereby inducing apoptosis of the endothelial cell.

For the purpose of the present disclosure, an endothelial cell-specific promoter can contain one or more cis-regulatory elements, which improve the endothelial cell-specificity of the promoters compared to the promoter without the cis-regulatory elements. In one example, the cis-regulatory element comprises an enhancer. In another aspect, the cis-regulatory element comprises a hypoxia response element. In other examples, the cis-regulatory element comprises both an enhancer and a hypoxia response element.

In one embodiment, a cis-regulatory element useful for the disclosure comprises a nucleotide sequence at least 60%, 70%, 80%, 90%, 95%, 96%, 97%, 98%, 99%, or 100% identical to SEQ ID NO: 11 or SEQ ID NO: 12 (the complementary sequence of SEQ ID NO: 11), wherein the cis-regulatory element improves endothelial cell specificity of a promoter compared to a promoter without the cis-regulatory element. The cis-regulatory element can further comprise an additional nucleotide sequence at least 60%, 70%, 80%, 90%, 95%, 96%, 97%, 98%, 99%, or 100% identical to SEQ ID NO: 13 or SEQ ID NO: 14 (the complementary sequence of SEQ ID NO: 13).

In another embodiment, a cis-regulatory element for the disclosure comprises a nucleotide sequence at least 60%, 70%, 80%, 90%, 95%, 96%, 97%, 98%, 99%, or 100% identical to SEQ ID NO: 13 or SEQ ID NO: 14 (the complementary sequence of SEQ ID NO: 13), wherein the cis-regulatory element improves endothelial cell specificity of a promoter compared to a promoter without the cis-regulatory element. The cis-regulatory element can further comprise an additional nucleotide sequence at least 60%, 70%, 80%, 90%, 95%, 96%, 97%, 98%, 99%, or 100% identical to SEQ ID NO: 11 or SEQ ID NO: 12 (the complementary sequence of SEQ ID NO: 11).

In other embodiments, a cis-regulatory element for the disclosure comprises a nucleotide sequence at least 60%, 70%, 80%, 90%, 95%, 96%, 97%, 98%, 99%, or 100% identical to SEQ ID NO: 15 or SEQ ID NO: 16 (the complementary sequence of SEQ ID NO: 15), wherein the cis-regulatory element improves endothelial cell specificity of a promoter compared to a promoter without the cis-regulatory element. In yet other embodiments, a cis-regulatory element for the nucleic acid construct comprises SEQ ID NO: 7 or any fragments, variants, derivatives, or analogs thereof, wherein the fragments, variants, derivatives, or analogs improve endothelial cell specificity of a promoter compared to a promoter without the cis-regulatory element.

In some embodiments, a cis-regulatory element for the disclosure comprises a nucleotide sequence at least 60%, 70%, 80%, 90%, 95%, 96%, 97%, 98%, 99%, or 100% identical to SEQ ID NO: 22 or SEQ ID NO: 23, wherein the cis-regulatory element improves endothelial cell specificity of a promoter compared to a promoter without the cis-regulatory element. In yet other embodiments, a cis-regulatory element for the nucleic acid construct comprises SEQ ID NO: 22 or SEQ ID NO: 23 or any fragments, variants, derivatives, or analogs thereof, wherein the fragments, variants, derivatives, or analogs improve endothelial cell specificity of a promoter compared to a promoter without the cis-regulatory element.

In other embodiments, a cis-regulatory element for the disclosure comprises a nucleotide sequence at least 60%, 70%, 80%, 90%, 95%, 96%, 97%, 98%, 99%, or 100% identical to SEQ ID NO: 24 or SEQ ID NO: 25, wherein the cis-regulatory element improves endothelial cell specificity of a promoter compared to a promoter without the cis-regulatory element. In yet other embodiments, a cis-regulatory element for the nucleic acid construct comprises SEQ ID NO: 24 or SEQ ID NO: 25 or any fragments, variants, derivatives, or analogs thereof, wherein the fragments, variants, derivatives, or analogs improve endothelial cell specificity of a promoter compared to a promoter without the cis-regulatory element.

Table 3 shows various cis-regulatory element sequences useful for the disclosure.

TABLE 3
Endothelial Cell-Specific Cis-regulatory Elements and Promoters

| SEQ ID | |
|---|---|
| NOs | Sequences |
| SEQ ID | ctggagggtg actttgcttc tggagccagt acttcatact tttcatt |
| NO: 11 | |
| SEQ ID | aatgaaaagt atgaagtact ggctccagaa gcaaagtcac cctccag |
| NO: 12 | |
| SEQ ID | gtacttcata cttttcattc caatggggtg actttgcttc tgga |
| NO: 13 | |
| SEQ ID | tccagaagca aagtcacccc attggaatga aaagtatgaa gtac |
| NO: 14 | |
| SEQ ID | 3X element |
| NO: 15 | ctccagaagcaaagtcaccccattggaatgaaaagtatgaagtacaatgaaaagtatgaagt |
| | actggctccagaagcaaagtcaccctccagaagcaaagtcaccccattggaatgaaaagtat |
| | gaagtac |
| SEQ ID | 3x element (Complementary Sequence of SEQ ID NO: 15) |
| NO: 16 | gtacttcatacttttcattccaatggggtgactttgcttctggagggtgactttgcttctgg |
| | agccagtacttcatacttttcattgtacttcatacttttcattccaatggggtgactttgct |
| | tctggag |
| SEQ ID | PPE-1 Promoter |
| NO: 17 | gtacgtgtacttctgatcggcgatactagggagataaggatgtgcctgacaaaaccacattg |
| | ttgttgttatcattattatttagttttccttccttgctaactcctgacggaatctttctcac |
| | ctcaaatgcgaagtactttagtttagaaaagacttggtggaaggggtggtggtggaaaagta |
| | gggtgatcttccaaactaatctggttccccgcccgccccagtagctgggattcaagagcgaa |
| | gagtggggatcgtccccttgtttgatcagaaagacataaaaggaaaatcaagtgaacaatga |
| | tcagccccacctccaccccacccccctgcgcgcgcacaatacaatctatttaattgtacttc |
| | atacttttcattccaatggggtgactttgcttctggagaaactcttgattcttgaactctgg |
| | ggctggcagctagcaaaaggggaagcgggctgctgctctctgcaggttctgcagcggtctct |
| | gtctagtgggtgttttctttttcttagccctgcccctggattgtcagacggcgggcgtctgc |
| | ctctgaagttagccgtgatttcctctagagccgggtcttatctctggctgcacgttgcctgt |
| | gggtgactaatcacacaataacattgtttagggctggaatgaagtcagagctgtttaccccc |
| | actctataggggttcaatataaaaaggcggcggagaactgtccgagtcagaagcgttcctgc |
| | accggcgctgagagcctgacccggtctgctccgctgtccttgcgcgctgcctcccggctgcc |
| | cgcgacgctttcgccccagtggaagggccacttgctgcggccgc |
| SEQ ID | PPE-1-3X promoter |
| NO: 18 | gtacgtgtacttctgatcggcgatactagggagataaggatgtgcctgacaaaaccacattg |
| | ttgttgttatcattattatttagttttccttccttgctaactcctgacggaatctttctcac |
| | ctcaaatgcgaagtactttagtttagaaaagacttggtggaaggggtggtggtggaaaagta |
| | gggtgatcttccaaactaatctggttccccgcccgccccagtagctgggattcaagagcgaa |
| | gagtggggatcgtccccttgtttgatcagaaagacataaaaggaaaatcaagtgaacaatga |
| | tcagccccacctccaccccacccccctgcgcgcgcacaatacaatctatttaattgtacttc |
| | atacttttcattccaatggggtgactttgcttctggagaaactcttgattcttgaactctgg |
| | ggctggcagctagcctccagaagcaaagtcaccccattggaatgaaaagtatgaagtacaat |
| | gaaaagtatgaagtactggctccagaagcaaagtcaccctccagaagcaaagtcaccccatt |
| | ggaatgaaaagtatgaagtacgctagcaaaaggggaagcgggctgctgctctctgcaggttc |
| | tgcagcggtctctgtctagtgggtgttttctttttcttagccctgcccctggattgtcagac |
| | ggcgggcgtctgcctctgaagttagccgtgatttcctctagagccgggtcttatctctggct |
| | gcacgttgcctgtgggtgactaatcacacaataacattgtttagggctggaatgaagtcaga |
| | gctgtttacccccactctataggggttcaatataaaaaggcggcggagaactgtccgagtca |
| | gaagcgttcctgcaccggcgctgagagcctgacccggtctgctccgctgtccttgcgcgctg |
| | cctcccggctgcccgcgacgctttcgccccagtggaagggccacttgctgcggccgc |
| SEQ ID | ggtgactttg cttctggag |
| NO: 22 | |
| SEQ ID | ctccagaagcaaagtcacc |
| NO: 23 | |
| SEQ ID | gtacttcata cttttcatt |
| NO: 24 | |
| SEQ ID | aatgaaaagtatgaagtac |
| NO: 25 | |
| SEQ ID | Hypoxia Response element |
| NO: 26 | gcacgt |

A cis-regulatory element for the present disclosure can be linked to a promoter upstream or downstream of the promoter or inserted between the two nucleotides in the promoter. The endothelial cell-specific promoter for the present disclosure can utilize any promoters known in the art. For example, suitable promoters which can be utilized for the present disclosure include the endothelial-specific promoters: preproendothelin-1 (PPE-1 promoter), US 2010/0282634, published Nov. 11, 2010; and WO 2011/083464, published Jul. 14, 2011); the PPE-1-3X promoter (U.S. Pat. Nos. 7,579,327, 8,071,740, 8,039,261, US2010/0282634, US 2007/0286845, WO 2011/083464, and WO2011/083466); the TIE-1 (S79347, S79346) and the TIE-2 (U53603) promoters [Sato T N, Proc Natl Acad Sci USA 1993 Oct. 15; 90(20):9355-8], the Endoglin promoter [Y11653; Rius C, Blood 1998 Dec. 15; 92(12):4677-90], the von Willerbrand factor [AF152417; Collins C J Proc Natl Acad Sci USA 1987 July; 84(13):4393-7], the KDR/flk-1 promoter [X89777, X89776; Ronicke V, Circ Res 1996 August; 79(2):277-85], The FLT-1 promoter [D64016 AJ224863; Morishita K,: J Biol Chem 1995 Nov. 17; 270(46):27948-53], the Egr-1 promoter [AJ245926; Sukhatme V P, Oncogene Res 1987 September-October; 1(4):343-55], the E-selectin promoter [Y12462; Collins T J Biol Chem 1991 Feb. 5; 266(4):2466-73], The endothelial adhesion molecules promoters: ICAM-1 [X84737; Horley K J EMBO J 1989 October; 8(10):2889-96], VCAM-1 [M92431; Iademarco M F, J Biol Chem 1992 Aug. 15; 267(23): 16323-9], PECAM-1 [AJ313330 X96849; CD31, Newman P J, Science 1990 Mar. 9; 247(4947): 1219-22], the vascular smooth-muscle-specific elements: CArG box X53154 and aortic carboxypeptidase-like protein (ACLP) promoter [AF332596; Layne M D, Circ Res. 2002; 90: 728-736] and Aortic Preferentially Expressed Gene-1 [Yen-Hsu Chen J. Biol. Chem, Vol. 276, Issue 50, 47658-47663, Dec. 14, 2001], all of which are incorporated herein by reference in their entireties.

In one embodiment, a promoter linked to the endothelial cell-specific element comprises a nucleotide sequence at least 60%, 70%, 80%, 90%, 95%, 96%, 97%, 98%, 99%, or 100% of SEQ ID NO: 17, wherein the promoter linked to the element induces endothelial cell-specificity to the gene operably linked to the promoter. In another embodiment, a promoter linked to the endothelial cell-specific element comprises a fragment, a variant, a derivative, or an analog of a wild-type PPE-1 promoter, wherein said fragment, variant, derivative, or analog thereof induces endothelial cell-specificity to the gene operably linked to the promoter. In one example, the endothelial cell-specific element can be inserted between nucleotide residues 442 and 449 corresponding to SEQ ID NO: 17.

In further embodiments, an endothelial cell-specific promoter comprises a hypoxia responsive element. A hypoxia responsive element (HRE) is located on the antisense strand of the endothelin-1 promoter. This element is a hypoxia-inducible factor-1 binding site that is required for positive regulation of the endothelin-1 promoter (of the human, rat and murine gene) by hypoxia. Hypoxia is a potent signal, inducing the expression of several genes including erythropoietin (Epo), VEGF, and various glycolytic enzymes. The core sequence (8 base pairs) is conserved in all genes that respond to hypoxic conditions and the flanking regions are different from other genes. The ET-I hypoxia responsive element is located between the GAT A-2 and the AP-1 binding sites. In one example, a hypoxia response element comprises SEQ ID NO: 26, a fragment, a variant, a derivative, or an analog thereof.

In other embodiments, an endothelial cell-specific promoter useful for the disclosure comprises, consists essentially of, or consists of a nucleotide sequence at least 60%, 70%, 80%, 90%, 95%, 96%, 97%, 98%, 99%, or 100% of SEQ ID NO: 18, wherein the promoter linked to the cis-regulatory element induces endothelial cell-specificity to the gene operably linked to the promoter. In another embodiment, an endothelial cell-specific promoter comprises a fragment, a variant, a derivative, or an analog of SEQ ID NO: 18, wherein said fragment, variant, derivative, or analog thereof induces endothelial cell-specificity to the gene operably linked to the promoter.

Additional variations of the endothelial cell-specific promoters can be found at WO2011/083464, WO2011/083466, and WO2012/052423, which are incorporated herein by reference in their entireties.

The present disclosure also provides a novel promoter sequence comprising a nucleotide sequence SEQ ID NO: 17. In one example, the promoter further comprises an endothelial cell-specific cis-regulatory element. In one example, the endothelial cell-specific cis-regulatory element comprises SEQ ID NO: 11, SEQ ID NO: 12, SEQ ID NO: 13, SEQ ID NO: 14, SEQ ID NO: 15, SEQ ID NO: 16, SEQ ID NO: 22, SEQ ID NO: 23, SEQ ID NO: 24, SEQ ID NO: 25, SEQ ID NO: 26 or any fragments, derivatives, variants, or analogs thereof, wherein the fragments, derivatives, variants, or analogs thereof improve endothelial cell-specificity of the promoter compared to a promoter without the cis-regulatory element. In another example, the promoter comprises a nucleotide sequence of SEQ ID NO: 18. The disclosure includes a nucleic acid construct comprising the novel promoter and a heterologous nucleotide sequence. In one embodiment, the heterologous nucleic acid sequence comprises a nucleotide sequence encoding a FAS-chimera protein described herein. In another embodiment, the heterologous nucleotide sequence comprises an adenovirus sequence.

C. Vector

The disclosure also provides a vector comprising the nucleic acid construct, which comprises a FAS-chimera gene operably linked to an endothelial cell-specific promoter. For the purposes of this disclosure, numerous vector systems can be employed. For example, various viral gene delivery systems that can be used in the practice of this aspect of the disclosure include, but are not limited to, an adenoviral vector, an alphavirus vector, an enterovirus vector, a pestivirus vector, a lentiviral vector, a baculoviral vector, a herpesvirus vector, an Epstein Barr viral vector, a papovaviral vector, a poxvirus vector, a vaccinia viral vector, an adeno-associated viral vector and a herpes simplex viral vector.

In another embodiment, a vector comprising a FAS-chimera gene operably linked to an endothelial cell-specific promoter is an adenovirus. For example, the adenovirus can be any one or more of human adenovirus species A (serotypes 12, 18, and 31), B (serotpyes 3, 7, 11, 14, 16, 21, 34, 35, 50, and 55), C (serotypes 1, 2, 5, 6, and 57), D (8, 9, 10, 13, 15, 17, 19, 20, 22-30, 32, 33, 36-39, 42-49, 51, 53, 54, and 56), E (serotype 4), F (serotype 40 and 41), or G (serotype 52). In a particular embodiment, the adenovirus for the disclosure is human adenovirus serotype 5. In some embodiments, the adenovirus useful for gene therapy is a recombinant non-replicating adenovirus, which does not contain an E1 region and an E3 region. In certain embodiments, the adenovirus for the disclosure is a conditionally replicating adenovirus, which does not contain an E3 region, but contains an E1 region.

In one embodiment, the vector comprises, consists essentially of, or consists of SEQ ID NO: 19. In another embodiment, the adenovirus vector is an isolated virus having European Collection of Cell Cultures (ECACC) Accession Number 13021201.

D. Biological Deposits

Biological deposits were made with the European Collection of Cell Cultures (ECACC) located at Health Protection Agency Culture Collections, Health Protection Agency, Microbiology Services, Porton Down, Salisbury, SP4 0JG, UK, pursuant to the Budapest Treaty and pursuant to 37 C.F.R. § 1.808. Samples of the deposited materials will become available to the public upon grant of a patent. The disclosure described and claimed herein is not to be limited by the scope of the strain deposited, since the deposited embodiment is intended only as an illustration of the disclosure.

$$\begin{matrix}\begin{matrix}Strain & \end{matrix} & ECACC Accession No . & Date Deposited \\ & & \\ VB \text{-} 111 & 13021201 & February 12 , 2013\end{matrix}$$

### III. VEGF Antagonists

VEGF, vascular endothelial growth factor, is an endothelial cell-specific mitogen and an inducer of angiogenesis. The term VEGF encompasses the members of the VEGF gene family: VEGF-A, VEGF-B, VEGF-C, and VEGF-D. VEGF-A is considered the prototype member of the VEGF gene family. Through alternative exon splicing, VEGF-A exists in four different isoforms: VEGF₁₂₁, VEGF₁₆₅, VEGF₁₈₉, and VEGF₂₀₆. The four VEGF-A isoforms are 121, 165, 189, and 206 amino acids in length (respectively) after signal sequence cleavage.

Once expressed, VEGF is secreted extracellularly where it binds to the extracellular region of a VEGF receptor (VEGFR). There are two primary VEGFRs, VEGFR-1 or VEGFR-2, both of which are receptor tyrosine kinases. A third VEGFR, VEGFR-3, is a related receptor tyrosine kinase that only binds VEGF-C and VEGF-D. Upon binding to VEGF, the VEGFRs signal downstream events that lead to endothelial cell proliferation and angiogenesis. VEGF-C and VEGF-D are known to regulate lymphatic angiogenesis.

The VEGF gene contains nucleotide sequences that are highly homologous to those of hypoxia-inducible factor-1 (HIF-1). These HIF-1 like sequences enable induction of VEGF gene expression under hypoxic conditions. Thus, under low oxygen conditions, such as within a tumor microenvironment, VEGF gene expression is induced. The production of high levels of VEGF within a tumor bed results in increased VEGFR signaling and thus endothelial cell growth and angiogenesis. The formation of new blood vessels within the tumor provides blood and oxygen to the growing tumor.

Due to the prominent role of VEGF in angiogenesis and tumor growth and development, VEGF antagonists are studied as potential cancer therapeutic agents. VEGF antagonists can prevent VEGF activity by binding directly to VEGF and blocking its interaction with a VEGFR. This reduces signaling from the VEGFR and downstream events, thereby causing a reduction in angiogenesis. In one embodiment, a VEGF antagonist useful for the disclosure is an anti-VEGF antibody or a VEGF binding molecule. In another embodiment, an anti-VEGF antibody or VEGF-binding molecule is a monoclonal antibody, a humanized antibody, a human antibody, a single chain antibody, or a chimeric antibody. In another embodiment, an anti-VEGF antibody or VEGF-binding molecule for the therapy comprises Fab, F(ab)₂, Fv, or scFv.

Another type of VEGF antagonist that can reduce or inhibit VEGF activity is a molecule binding to a VEGFR and thus blocking VEGFR interaction with VEGF. This interference of receptor/ligand binding prevents VEGFR signaling and reduces angiogenesis and endothelial cell proliferation. In one embodiment, the VEGF antagonist is an anti-VEGFR antibody or VEGFR-binding molecule. In another embodiment, the anti-VEGFR antibody or VEGFR-binding molecule is a monoclonal antibody, a humanized antibody, a human antibody, a single chain antibody, or a chimeric antibody. In another embodiment, the anti-VEGFR antibody or VEGFR-binding molecule comprises Fab, F(ab)₂, Fv, or scFv.

VEGF antagonists that bind to VEGF or VEGFR can inhibit VEGF activity by similar mechanisms of action in that they prevent receptor/ligand interaction, VEGFR signaling, and downstream signaling events such as endothelial cell proliferation and angiogenesis. Thus, in one embodiment, the VEGF antagonist is selected from the group consisting of bevacizumab (U.S. Pat. No. 7,169,901, incorporated herein by reference in its entirety), ranibizumab (U.S. Pat. No. 7,297,334, incorporated herein by reference in its entirety), VGX-100 (U.S. Pat. No. 7,423,125, incorporated herein by reference in its entirety), r84 (U.S. Pat. No. 8,034,905, incorporated herein by reference in its entirety), aflibercept (U.S. Pat. No. 5,952,199, incorporated herein by reference in its entirety), IMC-18F1 (U.S. Pat. No. 7,972,596, incorporated herein by reference in its entirety), IMC-1C11 (PCT/US2000/02180, incorporated herein by reference in its entirety), and ramucirumab (U.S. Pat. No. 7,498,414, incorporated herein by reference in its entirety). A VEGF binding molecule includes other forms of antibody derived molecules, e.g., a monobody, diabody, minibody, or any chimeric proteins comprising at least one CDR of a VEGF binding antibody, e.g., bevacizumab.

In one embodiment, the anti-VEGF antibody or VEGF binding molecule comprises at least one CDR selected from the group consisting of V_{H} CDR1 (SEQ ID NO: 28), V_{H} CDR2 (SEQ ID NO: 29), V_{H} CDR3 (SEQ ID NO: 30), V_{L} CDR1 (SEQ ID NO: 31), V_{L} CDR2 (SEQ ID NO: 32), V_{L} CDR3 (SEQ ID NO: 33), and any combination thereof. See Table 4.

TABLE 4
Amino Acid Sequences of Complementarity
Determining Regions

| CDR | Sequence |
|---|---|
| V_{H} CDR1 (SEQ ID NO: 28) | GYTFTNYGMN |
| V_{H} CDR2 (SEQ ID NO: 29) | WINTYTGEPTYAADFKR |
| V_{H} CDR3 (SEQ ID NO: 30) | YPHYYGSSHWYFDV |
| V_{L} CDR1 (SEQ ID NO: 31) | SASQDISNYLN |
| V_{L} CDR2 (SEQ ID NO: 32) | FTSSLHS |
| V_{L} CDR3 (SEQ ID NO: 33) | QQYSTVPWT |

In another embodiment, the anti-VEGF antibody or the VEGF binding molecule comprises CDR1 (SEQ ID NO: 28), CDR2 (SEQ ID NO: 29), or CDR3 (SEQ ID NO: 30) of the heavy chain variable region (V_{H}) of bevacizumab. For example, an anti-VEGF antibody or VEGF binding molecule comprises CDR1 and CRD2 of V_{H}, CDR 1 and CDR3 of V_{H}, CDR2 and CDR3 of V_{H}, or CDR1, CDR2, or CDR3 of V_{H}. In other embodiments, the anti-VEGF antibody or the VEGF binding molecule comprises CDR1 (SEQ ID NO: 31), CDR2 (SEQ ID NO: 32), or CDR3 (SEQ ID NO: 33) of the light chain variable region (V_{L}) of bevacizumab. For example, an anti-VEGF antibody or VEGF-binding molecule comprises CDR1 and CDR2 of V_{L}, CDR1 and CDR3 of V_{L}, CDR2 and CDR3 of V_{L}, or CDR1, CDR2, and CDR3 of V_{L}. In some embodiments, an anti-VEGF antibody or VEGF binding molecule comprises V_{H} of bevacizumab. In certain embodiments, an anti-VEGF antibody or VEGF binding molecule comprises V_{L} of bevacizumab.

In another aspect of the present disclosure, the anti-VEGF antibody or VEGF binding molecule comprises V_{H} CDR1 (SEQ ID NO: 28), V_{H} CDR2 (SEQ ID NO: 29), V_{H} CDR3 (SEQ ID NO: 30), V_{L} CDR1 (SEQ ID NO: 31), V_{L} CDR2 (SEQ ID NO: 32), and V_{L} CDR3 (SEQ ID NO: 33).

### IV. Treatment Methods Using Adenovirus Expressing Fas-Chimera Protein and a VEGF Antagonist

One embodiment of the present disclosure provides methods of treating a tumor in a subject who is capable of exhibiting a change in at least one plasma biomarker or cell surface biomarker after administration of at least one priming dose of a vector which comprises a Fas-chimera gene operably linked to an endothelial cell-specific promoter, the method comprising administering a therapeutically effective dose of the vector to the subject after the subject exhibits a change in at least one plasma biomarker or cell surface biomarker after the administration of the priming dose. In one aspect, the disclosure provides a method of treating a tumor in a subject in need thereof comprising administering to the subject at least one priming dose of a vector which comprises a Fas-chimera gene operably linked to an endothelial cell-specific promoter and administering to the subject a therapeutically effective dose of the vector, wherein the subject has a change in at least one plasma biomarker or cell surface biomarker after the administration of the priming dose.

In another aspect, the disclosure provides a method of treating a tumor in a subject in need thereof, the method comprising: (a) administering to the subject at least one priming dose of a vector which comprises a Fas-chimera gene operably linked to an endothelial cell-specific promoter; (b) determining the subject as having a change in at least one plasma biomarker or cell surface biomarker as a result of the administration of the priming dose in (a); and (c) administering a therapeutically effective dose of the vector to the subject having a change in at least one plasma biomarker or cell surface biomarker in (b). In another aspect, the disclosure provides a method of treating a tumor in a subject in need thereof, the method comprising: (a) administering to the subject at least one priming dose of a vector which comprises a Fas-chimera gene operably linked to an endothelial cell-specific promoter; (b) measuring serum levels of at least one plasma biomarker or cell surface biomarker of the subject after the administration of the priming dose; (c) determining the subject as having a change in at least one plasma biomarker or cell surface biomarker as a result of the administration of the priming dose in (a); and (d) administering a therapeutically effective dose of the vector to the subject having a change in at least one plasma biomarker or cell surface biomarker in (c).

In some aspects of the disclosure, the change in at least one plasma biomarker or cell surface biomarker of the subject is measured after at least one priming dose of the vector. In one aspect, the change in at least one plasma biomarker or cell surface biomarker of the subject is measured less than about 1 hour, less than about 2 hours, less than about 3 hours, less than about 4 hours, less than about 5 hours, less than about 6 hours, less than about 7 hours, less than about 8 hours, less than about 9 hours, less than about 12 hours, less than about 16 hours, less than about 20 hours, less than about 24 hours, less than about 48 hours, less than about 72 hours, less than about 96 hours, less than about 120 hours, and less than about 148 hours after administration of at least one priming dose of the vector.

In one aspect, the disclosure provides a method of treating a tumor in a subject in need thereof, wherein the method prolongs the median time to disease progression in subjects having a change in at least one plasma biomarker or cell surface biomarker after receiving at least one priming dose of the vector, compared to subjects not having a change in at least one plasma biomarker or cell surface biomarker after receiving at least one priming dose of the vector. In some aspects, the median time to disease progression in subjects having a change in at least one plasma biomarker or cell surface biomarker after receiving at least one priming dose of the vector can be at least about 30 days, at least about 60 days, at least about 90 days, at least about 120 days, at least about 150 days, at least about 180 days, at least about 210 days, at least about 240 days, at least about 270 days, at least about 300 days, at least about 330 days, at least about 360 days, at least about 390 days, at least about 420 days, at least about 450 days, at least about 480 days, at least about at least about 510 days, and at least about 540 days.

In another aspect, the disclosure provides a method of treating a tumor in a subject in need thereof, wherein the method prolongs the median time to death in subjects having a change in at least one plasma biomarker or cell surface biomarker after receiving at least one priming dose of the vector, compared to subjects not having a change in at least one plasma biomarker or cell surface biomarker after receiving at least one priming dose of the vector. In some aspects, median time to death in subjects having a change in at least one plasma biomarker or cell surface biomarker after receiving at least one priming dose of the vector can be at least about 150 days, at least about 180 days, at least about 210 days, at least about 240 days, at least about 270 days, at least about 300 days, at least about 330 days, at least about 360 days, at least about 390 days, at least about 420 days, at least about 450 days, at least about 480 days, at least about 510 days, at least about 540 days, at least about 570 days, at least about 600 days, at least about at least about 630 days, at least about 660 days, at least about 690 days, at least about 720 days, at least about 750 days, and at least about 780 days.

In some aspects of the disclosure the plasma biomarker or cell surface marker is selected from the group consisting of MCP-1, MIP-1, MIP-2, MIP-1α, MIP-1β, MIG, RANTES, IP-10, IL-1α, IL-1β, IL-2, IL-3, IL-4, IL-5, IL-6, IL-8, IL-9, IL-10, IL-12, IL-13, IL-15, IL-17α, LIF, TNF-α, TNF-β, VEGF, G-CSF, IFN-α, IFN-β, IFN-γ, M-CSF, IL-1Ra, eotaxin, CA-125, and a combination thereof.

In some aspects, the change in plasma biomarker or cell surface marker is an increase in the level of at least one plasma biomarker or cell surface marker. In some aspects, the plasma biomarker or cell surface marker that exhibits an increase in the level after the administration of the priming dose comprises at least one of the markers selected from the group consisting of IL-6, MCP-1, MIP-2, MIG, RANTES, MIP-1α, MIP-1α, IP-10, IL-2, IL-10, LIF, TNF-α, TGF-β1, VEGF, IL-17α, G-CSF, GM-CSF, IFN-γ, IL-1α, IL-1β, IL-12, IL-13, IL-15, IL-9, IL-22, IL-23, IL-35, M-CSF, IL-1Ra, and a combination thereof. In a particular aspect, the plasma biomarker or cell surface maker comprises TNF-α, IL-17α, MIP-1α, and IL-1Ra.

In some aspects of the disclosure, the change in plasma biomarker or cell surface marker is a decrease in the level at least one plasma biomarker or cell surface marker. In some aspects, the plasma biomarker or cell surface marker that exhibits a decrease in the level comprises at least one of the markers selected from IL-10, IL-4, IL-3, IL-5, IL-13, IL-2, LIF, TNF-α, CA-125, and a combination thereof.

In another aspect, the present disclosure includes a method of inhibiting or reducing angiogenesis in a tumor of a subject who is capable of exhibiting a change in at least one plasma biomarker or cell surface biomarker after administration of at least one priming dose of a vector which comprises a Fas-chimera gene operably linked to an endothelial cell-specific promoter, the method comprising administering a therapeutically effective dose of the vector to the subject after the subject exhibits a change in at least one plasma biomarker or cell surface biomarker after the administration of the priming dose. In one aspect, the disclosure provides a method of inhibiting or reducing angiogenesis in a tumor of a subject in need thereof comprising administering to the subject at least one priming dose of a vector which comprises a Fas-chimera gene operably linked to an endothelial cell-specific promoter and administering to the subject a therapeutically effective dose of the vector, wherein the subject has a change in at least one plasma biomarker or cell surface biomarker after the administration of the priming dose. In another aspect, the disclosure provides a method of inhibiting or reducing angiogenesis in a tumor in a subject in need thereof, the method comprising: (a) administering to the subject at least one priming dose of a vector which comprises a Fas-chimera gene operably linked to an endothelial cell-specific promoter; (b) determining the subject as having a change in at least one plasma biomarker or cell surface biomarker as a result of the administration of the priming dose in (a); and (c) administering a therapeutically effective dose of the vector to the subject having a change in at least one plasma biomarker or cell surface biomarker in (b). In another aspect, the disclosure provides a method of treating a tumor in a subject in need thereof, the method comprising: (a) administering to the subject at least one priming dose of a vector which comprises a Fas-chimera gene operably linked to an endothelial cell-specific promoter; (b) measuring serum levels of at least one plasma biomarker or cell surface biomarker of the subject after the administration of the priming dose; (c) determining the subject as having a change in at least one plasma biomarker or cell surface biomarker as a result of the administration of the priming dose in (a); and (d) administering a therapeutically effective dose of the vector to the subject having a change in at least one plasma biomarker or cell surface biomarker in (c).

In some aspects of the disclosure the plasma biomarker or cell surface marker is selected from the group consisting of MCP-1, MIP-1, MIP-2, MIP-1α, MIP-1β, MIG, RANTES, IP-10, IL-1α, IL-1β, IL-2, IL-3, IL-4, IL-5, IL-6, IL-8, IL-9, IL-10, IL-12, IL-13, IL-15, IL-17α, LIF, TNF-α, TNF-β, VEGF, G-CSF, IFN-α, IFN-β, IFN-γ, M-CSF, IL-1Ra, eotaxin, CA-125, and a combination thereof.

In some aspects, the change in plasma biomarker or cell surface marker is an increase in the level of at least one plasma biomarker or cell surface marker. In some aspects, the plasma biomarker or cell surface marker that exhibits an increase in the level after the administration of the priming dose comprises at least one of the markers selected from the group consisting of IL-6, MCP-1, MIP-2, MIG, RANTES, MIP-1α, MIP-1α, IP-10, IL-2, IL-10, LIF, TNF-α, TGF-β1, VEGF, IL-17α, G-CSF, GM-CSF, IFN-γ, IL-1α, IL-1β, IL-12, IL-13, IL-15, IL-9, IL-22, IL-23, IL-35, M-CSF, IL-1Ra, and a combination thereof. In a particular aspect, the plasma biomarker or cell surface maker comprises TNF-α, IL-17α, MIP-1α, and IL-1Ra.

In some aspects of the disclosure, the change in plasma biomarker or cell surface marker is a decrease in the level at least one plasma biomarker or cell surface marker. In some aspects, the plasma biomarker or cell surface marker that exhibits a decrease in the level comprises at least one of the markers selected from IL-10, IL-4, IL-3, IL-5, IL-13, IL-2, LIF, TNF-α, CA-125, and a combination thereof.

In other aspects, the present disclosure provides a method for identifying a responder to a Fas-chimera gene therapy, the method comprising administering to a subject having a tumor at least one priming dose of a vector which comprises a Fas-chimera gene operably linked to an endothelial cell-specific promoter, wherein the subject exhibits a change in at least one plasma biomarker or cell surface biomarker after the administration of the priming dose. In one aspect, the disclosure provides a method for identifying a responder to a Fas-chimera gene therapy, the method comprising administering to a subject having a tumor at least one priming dose of a vector which comprises a Fas-chimera gene operably linked to an endothelial cell-specific promoter and administering to the subject a therapeutically effective dose of the vector, wherein the subject exhibits a change in at least one plasma biomarker or cell surface biomarker after the administration of the priming dose. In another aspect, the disclosure provides a method for identifying a responder to a Fas-chimera gene therapy, the method comprising: (a) administering to a subject at least one priming dose of a vector which comprises a Fas-chimera gene operably linked to an endothelial cell-specific promoter; (b) identifying the subject as having a change in at least one plasma biomarker or cell surface biomarker as a result of the administration of the priming dose in (a); and (c) administering a therapeutically effective dose of the vector to the subject having a change in at least one plasma biomarker or cell surface biomarker in (b).

In other aspects, the present disclosure provides a method for identifying a responder to a Fas-chimera gene therapy, the method comprising administering to a subject having a tumor at least one priming dose of a vector which comprises a Fas-chimera gene operably linked to an endothelial cell-specific promoter, wherein the subject exhibits a plasma biomarker or a cell surface biomarker for fever after the administration of the priming dose. In one aspect, the disclosure provides a method for identifying a responder to a Fas-chimera gene therapy, the method comprising administering to a subject having a tumor at least one priming dose of a vector which comprises a Fas-chimera gene operably linked to an endothelial cell-specific promoter and administering to the subject a therapeutically effective dose of the vector, wherein the subject exhibits a plasma biomarker or a cell surface biomarker for fever after the administration of the priming dose. In another aspect, the disclosure provides a method for identifying a responder to a Fas-chimera gene therapy, the method comprising: (a) administering to a subject at least one priming dose of a vector which comprises a Fas-chimera gene operably linked to an endothelial cell-specific promoter; (b) identifying the subject as exhibiting a plasma biomarker or a cell surface biomarker for fever as a result of the administration of the priming dose in (a); and (c) administering a therapeutically effective dose of the vector to the subject having a febrile body temperature in (b).

In some embodiments, the plasma biomarker or cell surface biomarker is selected from the group consisting of MCP-1, MIP-1, MIP-2, MIP-1α, MIP-1β, MIG, RANTES, IP-10, IL-1α, IL-1β, IL-2, IL-3, IL-4, IL-5, IL-6, IL-8, IL-9, IL-10, IL-12, IL-13, IL-15, IL-17α, LIF, TNF-α, TNF-β, VEGF, G-CSF, IFN-α, IFN-β, IFN-γ, M-CSF, IL-1Ra, eotaxin, CA-125, and a combination thereof.

In some aspects, the change in plasma biomarker or cell surface marker is an increase in the level of at least one plasma biomarker or cell surface marker. In some aspects, the plasma biomarker or cell surface marker that exhibits an increase in the level after the administration of the priming dose comprises at least one of the markers selected from the group consisting of IL-6, MCP-1, MIP-2, MIG, RANTES, MIP-1α, MIP-1α, IP-10, IL-2, IL-10, LIF, TNF-α, TGF-β1, VEGF, IL-17α, G-CSF, GM-CSF, IFN-γ, IL-1α, IL-1β, IL-12, IL-13, IL-15, IL-9, IL-22, IL-23, IL-35, M-CSF, IL-1Ra, and a combination thereof. In a particular aspect, the plasma biomarker or cell surface maker comprises TNF-α, IL-17α, MIP-1α, and IL-1Ra.

In some aspects of the disclosure, the change in plasma biomarker or cell surface marker is a decrease in the level at least one plasma biomarker or cell surface marker. In some aspects, the plasma biomarker or cell surface marker that exhibits a decrease in the level comprises at least one of the markers selected from IL-10, IL-4, IL-3, IL-5, IL-13, IL-2, LIF, TNF-α, CA-125, and a combination thereof.

In one embodiment, the disclosure is directed to a method of inducing apoptosis of an endothelial cell in a tumor of a subject who is capable of exhibiting a change in at least one plasma biomarker or cell surface biomarker after administration of at least one priming dose of a vector which comprises a Fas-chimera gene operably linked to an endothelial cell-specific promoter, the method comprising administering a therapeutically effective dose of the vector to the subject after the subject exhibits a change in at least one plasma biomarker or cell surface biomarker after the administration of the priming dose. In one aspect, the disclosure provides a method of inducing apoptosis of an endothelial cell in a tumor in a subject in need thereof comprising administering to the subject at least one priming dose of a vector which comprises a Fas-chimera gene operably linked to an endothelial cell-specific promoter and administering to the subject a therapeutically effective dose of the vector, wherein the subject has a change in at least one plasma biomarker or cell surface biomarker after the administration of the priming dose. In another aspect, the disclosure provides a method of inducing apoptosis of an endothelial cell in a tumor in a subject in need thereof, the method comprising: (a) administering to the subject at least one priming dose of a vector which comprises a Fas-chimera gene operably linked to an endothelial cell-specific promoter; (b) determining the subject as having a change in at least one plasma biomarker or cell surface biomarker as a result of the administration of the priming dose in (a); and (c) administering a therapeutically effective dose of the vector to the subject having a change in at least one plasma biomarker or cell surface biomarker in (b). In another aspect, the disclosure provides a method of inducing apoptosis of an endothelial cell in a tumor in a subject in need thereof, the method comprising: (a) administering to the subject at least one priming dose of a vector which comprises a Fas-chimera gene operably linked to an endothelial cell-specific promoter; (b) measuring serum levels of at least one plasma biomarker or cell surface biomarker of the subject after the administration of the priming dose; (c) determining the subject as having a change in at least one plasma biomarker or cell surface biomarker as a result of the administration of the priming dose in (a); and (d) administering a therapeutically effective dose of the vector to the subject having a change in at least one plasma biomarker or cell surface biomarker in (c).

In certain embodiments, the disclosure provides a method of reducing the size of a tumor in a subject who is capable of exhibiting a change in at least one plasma biomarker or cell surface biomarker after administration of at least one priming dose of a vector which comprises a Fas-chimera gene operably linked to an endothelial cell-specific promoter, the method comprising administering a therapeutically effective dose of the vector to the subject after the subject exhibits a change in at least one plasma biomarker or cell surface biomarker after the administration of the priming dose. In one aspect, the disclosure provides a method of reducing the size of a tumor in a subject in need thereof comprising administering to the subject at least one priming dose of a vector which comprises a Fas-chimera gene operably linked to an endothelial cell-specific promoter and administering to the subject a therapeutically effective dose of the vector, wherein the subject has a change in at least one plasma biomarker or cell surface biomarker after the administration of the priming dose. In another aspect, the disclosure provides a method of reducing the size of a tumor in a subject in need thereof, the method comprising: (a) administering to the subject at least one priming dose of a vector which comprises a Fas-chimera gene operably linked to an endothelial cell-specific promoter; (b) determining the subject as having a change in at least one plasma biomarker or cell surface biomarker as a result of the administration of the priming dose in (a); and (c) administering a therapeutically effective dose of the vector to the subject having a change in at least one plasma biomarker or cell surface biomarker in (b). In another aspect, the disclosure provides a method of reducing the size of a tumor in a subject in need thereof, the method comprising: (a) administering to the subject at least one priming dose of a vector which comprises a Fas-chimera gene operably linked to an endothelial cell-specific promoter; (b) measuring serum levels of at least one plasma biomarker or cell surface biomarker of the subject after the administration of the priming dose; (c) determining the subject as having a change in at least one plasma biomarker or cell surface biomarker as a result of the administration of the priming dose in (a); and (d) administering a therapeutically effective dose of the vector to the subject having a change in at least one plasma biomarker or cell surface biomarker in (c).

In certain embodiments, the disclosure includes a method of treating a disease or condition associated with a tumor in a subject who is capable of exhibiting a change in at least one plasma biomarker or cell surface biomarker after administration of at least one priming dose of a vector which comprises a Fas-chimera gene operably linked to an endothelial cell-specific promoter, the method comprising administering a therapeutically effective dose of the vector to the subject after the subject exhibits a change in at least one plasma biomarker or cell surface biomarker after the administration of the priming dose. In one aspect, the disclosure provides a method of treating a disease or condition associated with a tumor in a subject in need thereof comprising administering to the subject at least one priming dose of a vector which comprises a Fas-chimera gene operably linked to an endothelial cell-specific promoter and administering to the subject a therapeutically effective dose of the vector, wherein the subject has a change in at least one plasma biomarker or cell surface biomarker after the administration of the priming dose. In another aspect, the disclosure provides a method of treating a disease or condition associated with a tumor in a subject in need thereof, the method comprising: (a) administering to the subject at least one priming dose of a vector which comprises a Fas-chimera gene operably linked to an endothelial cell-specific promoter; (b) determining the subject as having a change in at least one plasma biomarker or cell surface biomarker as a result of the administration of the priming dose in (a); and (c) administering a therapeutically effective dose of the vector to the subject having a change in at least one plasma biomarker or cell surface biomarker in (b). In another aspect, the disclosure provides a method of treating a disease or condition associated with a tumor in a subject in need thereof, the method comprising: (a) administering to the subject at least one priming dose of a vector which comprises a Fas-chimera gene operably linked to an endothelial cell-specific promoter; (b) measuring serum levels of at least one plasma biomarker or cell surface biomarker of the subject after the administration of the priming dose; (c) determining the subject as having a change in at least one plasma biomarker or cell surface biomarker as a result of the administration of the priming dose in (a); and (d) administering a therapeutically effective dose of the vector to the subject having a change in at least one plasma biomarker or cell surface biomarker in (c).

In some embodiments, the disclosure includes a method of identifying a candidate for Fas-chimera gene therapy comprising administering to a subject having a tumor at least one priming dose of a vector which comprises a Fas-chimera gene operably linked to an endothelial cell-specific promoter, wherein the subject exhibits a change in at least one plasma biomarker or cell surface biomarker after the administration of the priming dose. In one aspect, the disclosure provides a method of identifying a candidate for Fas-chimera gene therapy, the method comprising administering to a subject having a tumor at least one priming dose of a vector which comprises a Fas-chimera gene operably linked to an endothelial cell-specific promoter and administering to the subject a therapeutically effective dose of the vector, wherein the subject exhibits a change in at least one plasma biomarker or cell surface biomarker after the administration of the priming dose. In another aspect, the disclosure provides a method of identifying a candidate for Fas-chimera gene therapy, the method comprising: (a) administering to a subject at least one priming dose of a vector which comprises a Fas-chimera gene operably linked to an endothelial cell-specific promoter; (b) identifying the subject as having a change in at least one plasma biomarker or cell surface biomarker as a result of the administration of the priming dose in (a); and (c) administering a therapeutically effective dose of the vector to the subject having a change in at least one plasma biomarker or cell surface biomarker in (b).

In other embodiments, the disclosure includes a method of identifying a candidate for Fas-chimera gene therapy comprising administering to a subject having a tumor at least one priming dose of a vector which comprises a Fas-chimera gene operably linked to an endothelial cell-specific promoter, wherein the subject exhibits a plasma biomarker or cell surface biomarker for fever after the administration of the priming dose. In one aspect, the disclosure provides a method of identifying a candidate for Fas-chimera gene therapy, the method comprising administering to a subject having a tumor at least one priming dose of a vector which comprises a Fas-chimera gene operably linked to an endothelial cell-specific promoter and administering to the subject a therapeutically effective dose of the vector, wherein the subject exhibits a plasma biomarker or cell surface biomarker for fever after the administration of the priming dose. In another aspect, the disclosure provides a method of identifying a candidate for Fas-chimera gene therapy, the method comprising: (a) administering to a subject at least one priming dose of a vector which comprises a Fas-chimera gene operably linked to an endothelial cell-specific promoter; (b) identifying the subject as having a plasma biomarker or cell surface biomarker for fever as a result of the administration of the priming dose in (a); and (c) administering a therapeutically effective dose of the vector to the subject having a febrile body temperature in (b).

In some embodiments, the plasma biomarker or cell surface biomarker is selected from the group consisting of MCP-1, MIP-1, MIP-2, MIP-1α, MIP-1β, MIG, RANTES, IP-10, IL-1α, IL-1β, IL-2, IL-3, IL-4, IL-5, IL-6, IL-8, IL-9, IL-10, IL-12, IL-13, IL-15, IL-17α, LIF, TNF-α, TNF-β, VEGF, G-CSF, IFN-α, IFN-β, IFN-γ, M-CSF, IL-1Ra, eotaxin, CA-125, and a combination thereof.

In some aspects, the change in plasma biomarker or cell surface marker is an increase in the level of at least one plasma biomarker or cell surface marker. In some aspects, the plasma biomarker or cell surface marker that exhibits an increase in the level after the administration of the priming dose comprises at least one of the markers selected from the group consisting of IL-6, MCP-1, MIP-2, MIG, RANTES, MIP-1α, MIP-1α, IP-10, IL-2, IL-10, LIF, TNF-α, TGF-β1, VEGF, IL-17α, G-CSF, GM-CSF, IFN-γ, IL-1α, IL-1β, IL-12, IL-13, IL-15, IL-9, IL-22, IL-23, IL-35, M-CSF, IL-1Ra, and a combination thereof. In a particular aspect, the plasma biomarker or cell surface maker comprises TNF-α, IL-17α, MIP-1α, and IL-1Ra.

In some aspects of the disclosure, the change in plasma biomarker or cell surface marker is a decrease in the level at least one plasma biomarker or cell surface marker. In some aspects, the plasma biomarker or cell surface marker that exhibits a decrease in the level comprises at least one of the markers selected from IL-10, IL-4, IL-3, IL-5, IL-13, IL-2, LIF, TNF-α, CA-125, and a combination thereof.

In a particular embodiment, the growth or size of the tumor in a subject is measured by MRI. In another embodiment, the growth or size of the tumor is measured by CT scan. In other embodiments, the tumor in the subject is a recurrent tumor that arose during treatment with the vector. In yet other embodiments, the tumor in the subject is a metastatic tumor that arose during treatment with the vector.

In one aspect, the methods of the disclosure further comprise administration of an effective amount of a VEGF antagonist. In certain embodiments, the VEGF antagonist is selected from the group consisting of bevacizumab, ranibizumab, VGX-100, r84, aflibercept, IMC-18F1, IMC-1C11, and ramucirumab. In a particular embodiment, the VEGF antagonist is bevacizumab.

The term “subject” or “individual” or “animal” or “patient” or “mammal,” is meant any subject, particularly a mammalian subject, having or being expected to have increased or improved responsiveness to a vector expressing a FAS chimera protein. In some aspects, the term “subject” or “individual” or “animal” or “patient” or “mammal,” is meant any subject, particularly a mammalian subject, having or being expected to have increased or improved responsiveness to a combination regimen comprising a vector expressing a FAS chimera protein and a VEGF antagonist. In one embodiment, the subject is a human. In another embodiment, the subject is a cancer patient. In another embodiment, the subject is a female subject. In another embodiment, the subject is a male subject.

The phrase “subject or subject population” as used herein indicates that the subject or the subject population has been identified as a candidate or candidates for the Fas-chimera gene therapy or the combination therapy comprising Fas-chimera gene therapy and a VEGF antagonist. The subject or subject population can be identified as being a candidate or candidates for the Fas-chimera gene therapy or combination therapy prior to the administration of the vector or the VEGF antagonist or after administration of the vector or the VEGF antagonist.

In certain embodiments, identifying a candidate for the vector therapy or combination therapy comprises measuring various characteristics of tumor angiogenesis, for example, reduction in size of the tumor, inhibition of tumor growth, reduction in angiogenesis, reduction in neo-vascularization, or any known characteristics of angiogenesis. In other embodiments, identifying a candidate for the vector therapy or combination therapy comprises measuring plasma biomarkers or cell surface biomarkers. In some embodiments, the plasma biomarkers or cell surface biomarkers are associated with angiogenesis or fever. In certain embodiments, the biomarkers are selected from the group consisting of C-reactive protein (CRP), protein C, interleukin (IL)-6, IL-8, IL-10, IL-1β, TNF-α, sTNFRI, sTNFRII, monocyte chemotactic protein-1, ICAM-1, VEGF, FGF, and E-selectin. In other aspects, the plasma biomarker or cell surface biomarker is selected from the group consisting of MCP-1, MIP-1, MIP-2, MIP-1α, MIP-1β, MIG, RANTES, IP-10, IL-1α, IL-1β, IL-2, IL-3, IL-4, IL-5, IL-6, IL-8, IL-9, IL-10, IL-12, IL-13, IL-15, IL-17α, IL-22, IL-23, IL-35, LIF, TNF-α, TNF-β, TGF-β1, VEGF, G-CSF, IFN-α, IFN-β, IFN-γ, M-CSF, IL-1Ra, eotaxin, CA-125, and a combination thereof.

In certain aspects, once the subject or the subject population is identified as a candidate, the VEGF antagonist is administered prior to administering the vector, concomitantly with administration of a vector, or after administration of a vector. In other aspects, after a subject or a subject population is identified as a candidate, the vector is administered prior to the VEGF antagonist for at least one day earlier, at least two days earlier, at least three days earlier, at least four days earlier, at least five days earlier, at least six days earlier, at least seven days earlier, at least nine days earlier, at least 10 days earlier, at least two weeks earlier at least three weeks earlier, at least four weeks earlier, at least one month earlier, at least two months earlier, or more.

In one embodiment of the present disclosure, the disclosure includes a method of stabilizing a disease or disorder associated with cancer. In some embodiments, the disclosure includes a method of stabilizing a disease or disorder associated with metastatic colorectal cancer (mCRC), advanced nonsquamous non-small cell lung cancer (NSCLC), metastatic renal cell carcinoma (mRCC), glioblastoma multiforme (GBM), Müllerian cancer, ovarian cancer, peritoneal cancer, fallopian tube cancer, or uterine papillary serousspects, the present disclosure reduces the volume of malignant peritoneal fluid, e.g., ascites, reduces pain to the subject, prolongs survival of the subject, or any combinations thereof. The tumor that can be reduced, inhibited, or treated with the combination of the vector and the VEGF antagonist can be a solid tumor, a primary tumor, or a metastatic tumor. The term “metastatic” or “metastasis” refers to tumor cells that are able to establish secondary tumor lesions in another parts or organ.

A “solid tumor” includes, but is not limited to, sarcoma, melanoma, carcinoma, or other solid tumor cancer. “Sarcoma” refers to a tumor which is made up of a substance like the embryonic connective tissue and is generally composed of closely packed cells embedded in a fibrillar or homogeneous substance. Sarcomas include, but are not limited to, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” refers to a tumor arising from the melanocytic system of the skin and other organs. Melanomas include, for example, acra-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, metastatic melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidernoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma viflosum.

Additional cancers that can be inhibited or treated include, for example, Leukemia, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, papillary thyroid cancer, neuroblastoma, neuroendocrine cancer, esophageal cancer, genitourinary tract cancer, malignant hypercalcemia, cervical cancer, endometrial cancer, adrenal cortical cancer, prostate cancer, Müllerian cancer, ovarian cancer, peritoneal cancer, fallopian tube cancer, or uterine papillary serous carcinoma.

In other embodiments, the subject has had up to three, up to two, or up to one previous line of chemotherapy. In yet other embodiments, the subject has not had more than 3 prior lines of chemotherapy for recurrent cancer.

The priming dose or doses and the therapeutically effective dose or doses of the vector administered as part of the present disclosure can be measured in virus particles (VPs). In one embodiment, the priming dose is identical to the therapeutically effective dose. In another embodiment, the priming dose is lower than the therapeutically effective dose. In another embodiment, the priming dose is higher than the therapeutically effective dose. In one aspect, the priming dose is administered more than once. In another aspect, the priming dose is repeatedly administered. In one aspect, the therapeutically effective dose is administered more than once. In another aspect, the therapeutically effective dose is repeatedly administered.

In some embodiments, the priming dose of the vector is administered at an amount of less than about 1×10¹⁵, less than about 1×10¹⁴, less than about 5×10¹³, less than about 4×10¹³, less than about 3×10¹³, less than about 2×10¹³, less than about 1×10¹³, less than about 9×10¹², less than about 8×10¹², less than about 7×10¹², less than about 6×10¹², less than about 5×10¹², less than about 4×10¹², less than about 3×10¹², less than about 2×10¹², less than about 1×10¹², less than about 9×10¹¹, less than about 8×10¹¹, less than about 7×10¹¹, less than about 6×10¹¹, less than about 5×10¹¹, less than about 4×10¹¹, less than about 3×10¹¹, less than about 2×10¹¹, less than about 1×10¹¹, less than about 9×10¹⁰, less than about 8×10¹⁰, less than about 7×10¹⁰, less than about 6×10¹⁰, less than about 5×10¹⁰, less than about 4×10¹⁰, less than about 3×10¹⁰, less than about 2×10¹⁰, or less than about 1×10¹⁰ virus particles.

In other embodiments, the therapeutically effective dose of the vector is administered at an amount of at least about 1×10¹⁵, at least about 1×10¹⁴, at least about 5×10¹³, at least about 4×10¹³, at least about 3×10¹³, at least about 2×10¹³, at least about 1×10¹³, at least about 9×10¹², at least about 8×10¹², at least about 7×10¹², at least about 6×10¹², at least about 5×10¹², at least about 4×10¹², at least about 3×10¹², at least about 2×10¹², at least about 1×10¹², at least about 9×10¹¹, at least about 8×10¹¹, at least about 7×10¹¹, at least about 6×10¹¹, at least about 5×10¹¹, at least about 4×10¹¹, at least about 3×10¹¹, at least about 2×10¹¹, at least about 1×10¹¹, at least about 9×10¹⁰, at least about 8×10¹⁰, at least about 7×10¹⁰, at least about 6×10¹⁰, at least about 5×10¹⁰, at least about 4×10¹⁰, at least about 3×10¹⁰, at least about 2×10¹⁰, or at least about 1×10¹⁰ virus particles.

In one embodiment, the at least one priming dose and the at least one therapeutically effective dose of the vector in the combination therapy with bevacizumab is lower than the at least one priming dose and the at least one therapeutically effective dose used for the therapy without bevacizumab (e.g., a monotherapy therapy using the vector alone). For example, a therapeutically effective dose of the vector in the combination therapy with bevacizumab includes, but is not limited to equal to or less than about 1×10¹³, 9×10¹², 8×10¹², 7×10¹², 6×10¹², 5×10¹², 4×10¹², 3×10¹², 2×10¹², 1×10¹², 9×10¹¹, 8×10¹¹, 7×10¹¹, 6×10¹¹, 5×10¹¹, 4×10¹¹, 3×10¹¹, 2×10¹¹, 1×10¹¹, 9×10¹⁰, 8×10¹⁰, 7×10¹⁰, 6×10¹⁰, 5×10¹⁰, 4×10¹⁰, 3×10¹⁰, 2×10¹⁰, or 1×10¹⁰ virus particles. And, a priming dose of the vector in the combination therapy with bevacizumab includes, but is not limited to equal to or less than about 1×10¹³, 9×10¹², 8×10¹², 7×10¹², 6×10¹², 5×10¹², 4×10¹², 3×10¹², 2×10¹², 1×10¹², 9×10¹¹, 8×10¹¹, 7×10¹¹, 6×10¹¹, 5×10¹¹, 4×10¹¹, 3×10¹¹, 2×10¹¹, 1×10¹¹, 9×10¹⁰, 8×10¹⁰, 7×10¹⁰, 6×10¹⁰, 5×10¹⁰, 4×10¹⁰, 3×10¹⁰, 2×10¹⁰, or 1×10¹⁰ virus particles.

In one embodiment, the priming dose of the vector is administered at an amount of at least about 1×10¹¹ virus particles. In another embodiment, the priming dose of the vector is administered at an amount of at least about 1×10¹² virus particles. In another embodiment, the priming dose of the vector is administered at an amount of at least about 1×10¹³ virus particles. In another embodiment, the priming dose of the vector is administered at an amount of at least about 1×10¹⁴ virus particles. In other embodiments, the priming dose of the vector is administered at an amount of at least about 1×10⁷, 1×10⁸, 1×10⁹, 1×10¹⁰, or 5×10¹⁰ virus particles.

In another embodiment, the therapeutically effective dose of the vector is administered at an amount of at least about 1×10¹¹ virus particles. In another embodiment, the therapeutically effective dose of the vector is administered at an amount of at least about 1×10¹² virus particles. In another embodiment, the therapeutically effective dose of the vector is administered at an amount of at least about 1×10¹³ virus particles. In another embodiment, the therapeutically effective dose of the vector is administered at an amount of at least about 1×10¹⁴ virus particles. In other embodiments, the therapeutically effective dose of the vector is administered at an amount of at least about 1×10⁷, 1×10⁸, 1×10⁹, 1×10¹⁰, or 5×10¹⁰ virus particles.

The dose of the VEGF antagonist (e.g., bevacizumab) can be measured in mg/kg body weight. In one aspect, the dose of bevacizumab in the combination therapy with the vector is lower than the dose of bevacizumab without the vector (e.g., a therapy using bevacizumab alone). Non-limiting examples of an effective amount of bevacizumab include equal to or less than about 15 mg/kg, 14 mg/kg, 13 mg/kg, 12 mg/kg, 11 mg/kg, 10 mg/kg, 9 mg/kg, 8 mg/kg, 7 mg/kg, 6 mg/kg, 5 mg/kg, 4 mg/kg, 3 mg/kg, 2 mg/kg, or 1 mg/kg.

In a specific embodiment, the priming dose of the vector is administered at an amount of 3×10¹² to 1×10¹³ VPs, the therapeutically effective dose of the vector is administered at an amount of 3×10¹² to 1×10¹³ VPs, and bevacizumab is administered at an effective amount of 5 mg/kg to 15 mg/kg.

The present disclosure provides methods of treating a tumor in a subject in need thereof comprising administering at least one priming dose of the vector, at least one therapeutically effective dose of the vector, and a VEGF antagonist. The regimen used for administering the vector and the VEGF antagonist comprises repeated administration of the vector and the bevacizumab. In one embodiment, the vector is repeatedly administered every day, once in about 2 days, once in about 3 days, once in about 4 days, once in about 5 days, once in about 6 days, once in about 7 days, once in about 2 weeks, once in about 3 weeks, once in about 4 weeks, once in about 5 weeks, once in about 6 weeks, once in about 7 weeks, once in about 2 months, or once in about 6 months. In another embodiment the bevacizumab is repeatedly administered once in about 7 days, once in about 2 weeks, once in about 3 weeks, once in about 4 weeks, once in about 2 months, once in about 3 months, once in about 4 months, once in about 5 months, or once in about 6 months. In a particular embodiment, the vector is administered every 2 months and bevacizumab is administered every 2 weeks.

### V. Taxanes

Taxanes are diterpenes produced by the plants of the genus Taxus (yews), and are widely used as chemotherapy agents. Taxane can also be synthesized artificially. Taxanes act on microtubule function and inhibit mitosis. Taxane agents include, but are not limited to, paclitaxel (TAXOL®) and docetaxel (TAXOTERE®).

In one aspect, taxane can be fused to or bound to a heterologous moiety. Such a heterologous moiety can improve solubility of taxane formulation or reduce toxicity of taxane. For example, taxane can be fused to or bound to albumin: albumin-bound paclitaxel (ABRAXANE®, also called nab-paclitaxel) is an alternative formulation where paclitaxel is bound to albumin nano-particles.

Synthetic approaches to paclitaxel production led to the development of docetaxel. Docetaxel has a similar set of clinical uses to paclitaxel and is marketed under the name of TAXOTERE®.

In another aspect, taxane useful for the present invention includes, but is not limited to, paclitaxel, 10-deacetylbaccatin III, baccatin III, paclitaxel C, and 7-epipaclitaxel in the shells and leaves of hazel plants.

### IV. Treatment Methods Using Adenovirus Expressing Fas-Chimera Protein and One or More Chemotherapeutic Agents

One embodiment of the present disclosure provides methods of treating a tumor in a subject who is capable of exhibiting a change in at least one plasma biomarker or cell surface biomarker after administration of at least one priming dose of a vector which comprises a Fas-chimera gene operably linked to an endothelial cell-specific promoter, the method comprising administering a therapeutically effective dose of the vector to the subject after the subject exhibits a change in at least one plasma biomarker or cell surface biomarker after the administration of the priming dose. In one aspect, the disclosure provides a method of treating a tumor in a subject in need thereof comprising administering to the subject at least one priming dose of a vector which comprises a Fas-chimera gene operably linked to an endothelial cell-specific promoter and administering to the subject a therapeutically effective dose of the vector, wherein the subject has a change in at least one plasma biomarker or cell surface biomarker after the administration of the priming dose.

In another aspect, the disclosure provides a method of treating a tumor in a subject in need thereof, the method comprising: (a) administering to the subject at least one priming dose of a vector which comprises a Fas-chimera gene operably linked to an endothelial cell-specific promoter; (b) determining the subject as having a change in at least one plasma biomarker or cell surface biomarker as a result of the administration of the priming dose in (a); and (c) administering a therapeutically effective dose of the vector to the subject having a change in at least one plasma biomarker or cell surface biomarker in (b). In another aspect, the disclosure provides a method of treating a tumor in a subject in need thereof, the method comprising: (a) administering to the subject at least one priming dose of a vector which comprises a Fas-chimera gene operably linked to an endothelial cell-specific promoter; (b) measuring serum levels of at least one plasma biomarker or cell surface biomarker of the subject after the administration of the priming dose; (c) determining the subject as having a change in at least one plasma biomarker or cell surface biomarker as a result of the administration of the priming dose in (a); and (d) administering a therapeutically effective dose of the vector to the subject having a change in at least one plasma biomarker or cell surface biomarker in (c).

In some aspects of the disclosure, the change in at least one plasma biomarker or cell surface biomarker of the subject is measured after at least one priming dose of the vector. In one aspect, the change in at least one plasma biomarker or cell surface biomarker of the subject is measured less than about 1 hour, less than about 2 hours, less than about 3 hours, less than about 4 hours, less than about 5 hours, less than about 6 hours, less than about 7 hours, less than about 8 hours, less than about 9 hours, less than about 12 hours, less than about 16 hours, less than about 20 hours, less than about 24 hours, less than about 48 hours, less than about 72 hours, less than about 96 hours, less than about 120 hours, or less than about 148 hours after administration of at least one priming dose of the vector.

In one aspect, the disclosure provides a method of treating a tumor in a subject in need thereof, wherein the method prolongs the median time to disease progression in subjects having a change in at least one plasma biomarker or cell surface biomarker after receiving at least one priming dose of the vector, compared to subjects not having a change in at least one plasma biomarker or cell surface biomarker after receiving at least one priming dose of the vector. In some aspects, the median time to disease progression in subjects having a change in at least one plasma biomarker or cell surface biomarker after receiving at least one priming dose of the vector can be at least about 30 days, at least about 60 days, at least about 90 days, at least about 120 days, at least about 150 days, at least about 180 days, at least about 210 days, at least about 240 days, at least about 270 days, at least about 300 days, at least about 330 days, at least about 360 days, at least about 390 days, at least about 420 days, at least about 450 days, at least about 480 days, at least about at least about 510 days, or at least about 540 days.

In another aspect, the disclosure provides a method of treating a tumor in a subject in need thereof, wherein the method prolongs the median time to death in subjects having a change in at least one plasma biomarker or cell surface biomarker after receiving at least one priming dose of the vector, compared to subjects not having a change in at least one plasma biomarker or cell surface biomarker after receiving at least one priming dose of the vector. In some aspects, median time to death in subjects having a change in at least one plasma biomarker or cell surface biomarker after receiving at least one priming dose of the vector can be at least about 150 days, at least about 180 days, at least about 210 days, at least about 240 days, at least about 270 days, at least about 300 days, at least about 330 days, at least about 360 days, at least about 390 days, at least about 420 days, at least about 450 days, at least about 480 days, at least about 510 days, at least about 540 days, at least about 570 days, at least about 600 days, at least about at least about 630 days, at least about 660 days, at least about 690 days, at least about 720 days, at least about 750 days, or at least about 780 days.

In some aspects of the disclosure the plasma biomarker or cell surface marker is selected from the group consisting of MCP-1, MIP-1, MIP-2, MIP-1α, MIP-1β, MIG, RANTES, IP-10, IL-1α, IL-1β, IL-2, IL-3, IL-4, IL-5, IL-6, IL-8, IL-9, IL-10, IL-12, IL-13, IL-15, IL-17α, LIF, TNF-α, TNF-β, VEGF, G-CSF, IFN-α, IFN-β, IFN-γ, M-CSF, IL-1Ra, eotaxin, CA-125, and a combination thereof.

In some aspects, the change in plasma biomarker or cell surface marker is an increase in the level of at least one plasma biomarker or cell surface marker. In some aspects, the plasma biomarker or cell surface marker that exhibits an increase in the level after the administration of the priming dose comprises at least one of the markers selected from the group consisting of IL-6, MCP-1, MIP-2, MIG, RANTES, MIP-1α, MIP-1α, IP-10, IL-2, IL-10, LIF, TNF-α, TGF-β1, VEGF, IL-17α, G-CSF, GM-CSF, IFN-γ, IL-1α, IL-1β, IL-12, IL-13, IL-15, IL-9, IL-22, IL-23, IL-35, M-CSF, IL-1Ra, and a combination thereof. In a particular aspect, the plasma biomarker or cell surface maker comprises TNF-α, IL-17α, MIP-1α, and IL-1Ra.

In some aspects of the disclosure, the change in plasma biomarker or cell surface marker is a decrease in the level at least one plasma biomarker or cell surface marker. In some aspects, the plasma biomarker or cell surface marker that exhibits a decrease in the level comprises at least one of the markers selected from IL-10, IL-4, IL-3, IL-5, IL-13, IL-2, LIF, TNF-α, CA-125, and a combination thereof.

In another aspect, the present disclosure includes a method of inhibiting or reducing angiogenesis in a tumor of a subject who is capable of exhibiting a change in at least one plasma biomarker or cell surface biomarker after administration of at least one priming dose of a vector which comprises a Fas-chimera gene operably linked to an endothelial cell-specific promoter, the method comprising administering a therapeutically effective dose of the vector to the subject after the subject exhibits a change in at least one plasma biomarker or cell surface biomarker after the administration of the priming dose. In one aspect, the disclosure provides a method of inhibiting or reducing angiogenesis in a tumor of a subject in need thereof comprising administering to the subject at least one priming dose of a vector which comprises a Fas-chimera gene operably linked to an endothelial cell-specific promoter and administering to the subject a therapeutically effective dose of the vector, wherein the subject has a change in at least one plasma biomarker or cell surface biomarker after the administration of the priming dose. In another aspect, the disclosure provides a method of inhibiting or reducing angiogenesis in a tumor in a subject in need thereof, the method comprising: (a) administering to the subject at least one priming dose of a vector which comprises a Fas-chimera gene operably linked to an endothelial cell-specific promoter; (b) determining the subject as having a change in at least one plasma biomarker or cell surface biomarker as a result of the administration of the priming dose in (a); and (c) administering a therapeutically effective dose of the vector to the subject having a change in at least one plasma biomarker or cell surface biomarker in (b). In another aspect, the disclosure provides a method of treating a tumor in a subject in need thereof, the method comprising: (a) administering to the subject at least one priming dose of a vector which comprises a Fas-chimera gene operably linked to an endothelial cell-specific promoter; (b) measuring serum levels of at least one plasma biomarker or cell surface biomarker of the subject after the administration of the priming dose; (c) determining the subject as having a change in at least one plasma biomarker or cell surface biomarker as a result of the administration of the priming dose in (a); and (d) administering a therapeutically effective dose of the vector to the subject having a change in at least one plasma biomarker or cell surface biomarker in (c).

In some aspects of the disclosure the plasma biomarker or cell surface marker is selected from the group consisting of MCP-1, MIP-1, MIP-2, MIP-1α, MIP-1β, MIG, RANTES, IP-10, IL-1α, IL-1β, IL-2, IL-3, IL-4, IL-5, IL-6, IL-8, IL-9, IL-10, IL-12, IL-13, IL-15, IL-17α, LIF, TNF-α, TNF-β, VEGF, G-CSF, IFN-α, IFN-β, IFN-γ, M-CSF, IL-1Ra, eotaxin, CA-125, and a combination thereof.

In some aspects, the change in plasma biomarker or cell surface marker is an increase in the level of at least one plasma biomarker or cell surface marker. In some aspects, the plasma biomarker or cell surface marker that exhibits an increase in the level after the administration of the priming dose comprises at least one of the markers selected from the group consisting of IL-6, MCP-1, MIP-2, MIG, RANTES, MIP-1α, MIP-1α, IP-10, IL-2, IL-10, LIF, TNF-α, TGF-β1, VEGF, IL-17α, G-CSF, GM-CSF, IFN-γ, IL-1α, IL-1β, IL-12, IL-13, IL-15, IL-9, IL-22, IL-23, IL-35, M-CSF, IL-1Ra, and a combination thereof. In a particular aspect, the plasma biomarker or cell surface maker comprises TNF-α, IL-17α, MIP-1α, and IL-1Ra.

In some aspects of the disclosure, the change in plasma biomarker or cell surface marker is a decrease in the level at least one plasma biomarker or cell surface marker. In some aspects, the plasma biomarker or cell surface marker that exhibits a decrease in the level comprises at least one of the markers selected from IL-10, IL-4, IL-3, IL-5, IL-13, IL-2, LIF, TNF-α, CA-125, and a combination thereof.

In other aspects, the present disclosure provides a method for identifying a responder to a Fas-chimera gene therapy, the method comprising administering to a subject having a tumor at least one priming dose of a vector which comprises a Fas-chimera gene operably linked to an endothelial cell-specific promoter, wherein the subject exhibits a change in at least one plasma biomarker or cell surface biomarker after the administration of the priming dose. In one aspect, the disclosure provides a method for identifying a responder to a Fas-chimera gene therapy, the method comprising administering to a subject having a tumor at least one priming dose of a vector which comprises a Fas-chimera gene operably linked to an endothelial cell-specific promoter and administering to the subject a therapeutically effective dose of the vector, wherein the subject exhibits a change in at least one plasma biomarker or cell surface biomarker after the administration of the priming dose. In another aspect, the disclosure provides a method for identifying a responder to a Fas-chimera gene therapy, the method comprising: (a) administering to a subject at least one priming dose of a vector which comprises a Fas-chimera gene operably linked to an endothelial cell-specific promoter; (b) identifying the subject as having a change in at least one plasma biomarker or cell surface biomarker as a result of the administration of the priming dose in (a); and (c) administering a therapeutically effective dose of the vector to the subject having a change in at least one plasma biomarker or cell surface biomarker in (b).

In other aspects, the present disclosure provides a method for identifying a responder to a Fas-chimera gene therapy, the method comprising administering to a subject having a tumor at least one priming dose of a vector which comprises a Fas-chimera gene operably linked to an endothelial cell-specific promoter, wherein the subject exhibits a plasma biomarker or a cell surface biomarker for fever after the administration of the priming dose. In one aspect, the disclosure provides a method for identifying a responder to a Fas-chimera gene therapy, the method comprising administering to a subject having a tumor at least one priming dose of a vector which comprises a Fas-chimera gene operably linked to an endothelial cell-specific promoter and administering to the subject a therapeutically effective dose of the vector, wherein the subject exhibits a plasma biomarker or a cell surface biomarker for fever after the administration of the priming dose. In another aspect, the disclosure provides a method for identifying a responder to a Fas-chimera gene therapy, the method comprising: (a) administering to a subject at least one priming dose of a vector which comprises a Fas-chimera gene operably linked to an endothelial cell-specific promoter; (b) identifying the subject as exhibiting a plasma biomarker or a cell surface biomarker for fever as a result of the administration of the priming dose in (a); and (c) administering a therapeutically effective dose of the vector to the subject having a febrile body temperature in (b).

In some embodiments, the plasma biomarker or cell surface biomarker is selected from the group consisting of MCP-1, MIP-1, MIP-2, MIP-1α, MIP-1β, MIG, RANTES, IP-10, IL-1α, IL-1β, IL-2, IL-3, IL-4, IL-5, IL-6, IL-8, IL-9, IL-10, IL-12, IL-13, IL-15, IL-17α, LIF, TNF-α, TNF-β, VEGF, G-CSF, IFN-α, IFN-β, IFN-γ, M-CSF, IL-1Ra, eotaxin, CA-125, and a combination thereof.

In some aspects, the change in plasma biomarker or cell surface marker is an increase in the level of at least one plasma biomarker or cell surface marker. In some aspects, the plasma biomarker or cell surface marker that exhibits an increase in the level after the administration of the priming dose comprises at least one of the markers selected from the group consisting of IL-6, MCP-1, MIP-2, MIG, RANTES, MIP-1α, MIP-1α, IP-10, IL-2, IL-10, LIF, TNF-α, TGF-β1, VEGF, IL-17α, G-CSF, GM-CSF, IFN-γ, IL-1α, IL-1β, IL-12, IL-13, IL-15, IL-9, IL-22, IL-23, IL-35, M-CSF, IL-1Ra, and a combination thereof. In a particular aspect, the plasma biomarker or cell surface maker comprises TNF-α, IL-17α, MIP-1α, and IL-1Ra.

In some aspects of the disclosure, the change in plasma biomarker or cell surface marker is a decrease in the level at least one plasma biomarker or cell surface marker. In some aspects, the plasma biomarker or cell surface marker that exhibits a decrease in the level comprises at least one of the markers selected from IL-10, IL-4, IL-3, IL-5, IL-13, IL-2, LIF, TNF-α, CA-125, and a combination thereof.

In one embodiment, the disclosure is directed to a method of inducing apoptosis of an endothelial cell in a tumor of a subject who is capable of exhibiting a change in at least one plasma biomarker or cell surface biomarker after administration of at least one priming dose of a vector which comprises a Fas-chimera gene operably linked to an endothelial cell-specific promoter, the method comprising administering a therapeutically effective dose of the vector to the subject after the subject exhibits a change in at least one plasma biomarker or cell surface biomarker after the administration of the priming dose. In one aspect, the disclosure provides a method of inducing apoptosis of an endothelial cell in a tumor in a subject in need thereof comprising administering to the subject at least one priming dose of a vector which comprises a Fas-chimera gene operably linked to an endothelial cell-specific promoter and administering to the subject a therapeutically effective dose of the vector, wherein the subject has a change in at least one plasma biomarker or cell surface biomarker after the administration of the priming dose. In another aspect, the disclosure provides a method of inducing apoptosis of an endothelial cell in a tumor in a subject in need thereof, the method comprising: (a) administering to the subject at least one priming dose of a vector which comprises a Fas-chimera gene operably linked to an endothelial cell-specific promoter; (b) determining the subject as having a change in at least one plasma biomarker or cell surface biomarker as a result of the administration of the priming dose in (a); and (c) administering a therapeutically effective dose of the vector to the subject having a change in at least one plasma biomarker or cell surface biomarker in (b). In another aspect, the disclosure provides a method of inducing apoptosis of an endothelial cell in a tumor in a subject in need thereof, the method comprising: (a) administering to the subject at least one priming dose of a vector which comprises a Fas-chimera gene operably linked to an endothelial cell-specific promoter; (b) measuring serum levels of at least one plasma biomarker or cell surface biomarker of the subject after the administration of the priming dose; (c) determining the subject as having a change in at least one plasma biomarker or cell surface biomarker as a result of the administration of the priming dose in (a); and (d) administering a therapeutically effective dose of the vector to the subject having a change in at least one plasma biomarker or cell surface biomarker in (c).

In certain embodiments, the disclosure provides a method of reducing the size of a tumor in a subject who is capable of exhibiting a change in at least one plasma biomarker or cell surface biomarker after administration of at least one priming dose of a vector which comprises a Fas-chimera gene operably linked to an endothelial cell-specific promoter, the method comprising administering a therapeutically effective dose of the vector to the subject after the subject exhibits a change in at least one plasma biomarker or cell surface biomarker after the administration of the priming dose. In one aspect, the disclosure provides a method of reducing the size of a tumor in a subject in need thereof comprising administering to the subject at least one priming dose of a vector which comprises a Fas-chimera gene operably linked to an endothelial cell-specific promoter and administering to the subject a therapeutically effective dose of the vector, wherein the subject has a change in at least one plasma biomarker or cell surface biomarker after the administration of the priming dose. In another aspect, the disclosure provides a method of reducing the size of a tumor in a subject in need thereof, the method comprising: (a) administering to the subject at least one priming dose of a vector which comprises a Fas-chimera gene operably linked to an endothelial cell-specific promoter; (b) determining the subject as having a change in at least one plasma biomarker or cell surface biomarker as a result of the administration of the priming dose in (a); and (c) administering a therapeutically effective dose of the vector to the subject having a change in at least one plasma biomarker or cell surface biomarker in (b). In another aspect, the disclosure provides a method of reducing the size of a tumor in a subject in need thereof, the method comprising: (a) administering to the subject at least one priming dose of a vector which comprises a Fas-chimera gene operably linked to an endothelial cell-specific promoter; (b) measuring serum levels of at least one plasma biomarker or cell surface biomarker of the subject after the administration of the priming dose; (c) determining the subject as having a change in at least one plasma biomarker or cell surface biomarker as a result of the administration of the priming dose in (a); and (d) administering a therapeutically effective dose of the vector to the subject having a change in at least one plasma biomarker or cell surface biomarker in (c).

In certain embodiments, the disclosure includes a method of treating a disease or condition associated with a tumor in a subject who is capable of exhibiting a change in at least one plasma biomarker or cell surface biomarker after administration of at least one priming dose of a vector which comprises a Fas-chimera gene operably linked to an endothelial cell-specific promoter, the method comprising administering a therapeutically effective dose of the vector to the subject after the subject exhibits a change in at least one plasma biomarker or cell surface biomarker after the administration of the priming dose. In one aspect, the disclosure provides a method of treating a disease or condition associated with a tumor in a subject in need thereof comprising administering to the subject at least one priming dose of a vector which comprises a Fas-chimera gene operably linked to an endothelial cell-specific promoter and administering to the subject a therapeutically effective dose of the vector, wherein the subject has a change in at least one plasma biomarker or cell surface biomarker after the administration of the priming dose. In another aspect, the disclosure provides a method of treating a disease or condition associated with a tumor in a subject in need thereof, the method comprising: (a) administering to the subject at least one priming dose of a vector which comprises a Fas-chimera gene operably linked to an endothelial cell-specific promoter; (b) determining the subject as having a change in at least one plasma biomarker or cell surface biomarker as a result of the administration of the priming dose in (a); and (c) administering a therapeutically effective dose of the vector to the subject having a change in at least one plasma biomarker or cell surface biomarker in (b). In another aspect, the disclosure provides a method of treating a disease or condition associated with a tumor in a subject in need thereof, the method comprising: (a) administering to the subject at least one priming dose of a vector which comprises a Fas-chimera gene operably linked to an endothelial cell-specific promoter; (b) measuring serum levels of at least one plasma biomarker or cell surface biomarker of the subject after the administration of the priming dose; (c) determining the subject as having a change in at least one plasma biomarker or cell surface biomarker as a result of the administration of the priming dose in (a); and (d) administering a therapeutically effective dose of the vector to the subject having a change in at least one plasma biomarker or cell surface biomarker in (c).

In some embodiments, the disclosure includes a method of identifying a candidate for Fas-chimera gene therapy comprising administering to a subject having a tumor at least one priming dose of a vector which comprises a Fas-chimera gene operably linked to an endothelial cell-specific promoter, wherein the subject exhibits a change in at least one plasma biomarker or cell surface biomarker after the administration of the priming dose. In one aspect, the disclosure provides a method of identifying a candidate for Fas-chimera gene therapy, the method comprising administering to a subject having a tumor at least one priming dose of a vector which comprises a Fas-chimera gene operably linked to an endothelial cell-specific promoter and administering to the subject a therapeutically effective dose of the vector, wherein the subject exhibits a change in at least one plasma biomarker or cell surface biomarker after the administration of the priming dose. In another aspect, the disclosure provides a method of identifying a candidate for Fas-chimera gene therapy, the method comprising: (a) administering to a subject at least one priming dose of a vector which comprises a Fas-chimera gene operably linked to an endothelial cell-specific promoter; (b) identifying the subject as having a change in at least one plasma biomarker or cell surface biomarker as a result of the administration of the priming dose in (a); and (c) administering a therapeutically effective dose of the vector to the subject having a change in at least one plasma biomarker or cell surface biomarker in (b).

In other embodiments, the disclosure includes a method of identifying a candidate for Fas-chimera gene therapy comprising administering to a subject having a tumor at least one priming dose of a vector which comprises a Fas-chimera gene operably linked to an endothelial cell-specific promoter, wherein the subject exhibits a plasma biomarker or cell surface biomarker for fever after the administration of the priming dose. In one aspect, the disclosure provides a method of identifying a candidate for Fas-chimera gene therapy, the method comprising administering to a subject having a tumor at least one priming dose of a vector which comprises a Fas-chimera gene operably linked to an endothelial cell-specific promoter and administering to the subject a therapeutically effective dose of the vector, wherein the subject exhibits a plasma biomarker or cell surface biomarker for fever after the administration of the priming dose. In another aspect, the disclosure provides a method of identifying a candidate for Fas-chimera gene therapy, the method comprising: (a) administering to a subject at least one priming dose of a vector which comprises a Fas-chimera gene operably linked to an endothelial cell-specific promoter; (b) identifying the subject as having a plasma biomarker or cell surface biomarker for fever as a result of the administration of the priming dose in (a); and (c) administering a therapeutically effective dose of the vector to the subject having a febrile body temperature in (b).

In some embodiments, the plasma biomarker or cell surface biomarker is selected from the group consisting of MCP-1, MIP-1, MIP-2, MIP-1α, MIP-1β, MIG, RANTES, IP-10, IL-1α, IL-1β, IL-2, IL-3, IL-4, IL-5, IL-6, IL-8, IL-9, IL-10, IL-12, IL-13, IL-15, IL-17α, LIF, TNF-α, TNF-β, VEGF, G-CSF, IFN-α, IFN-β, IFN-γ, M-CSF, IL-1Ra, eotaxin, CA-125, and a combination thereof.

In some aspects, the change in plasma biomarker or cell surface marker is an increase in the level of at least one plasma biomarker or cell surface marker. In some aspects, the plasma biomarker or cell surface marker that exhibits an increase in the level after the administration of the priming dose comprises at least one of the markers selected from the group consisting of IL-6, MCP-1, MIP-2, MIG, RANTES, MIP-1α, MIP-1α, IP-10, IL-2, IL-10, LIF, TNF-α, TGF-β1, VEGF, IL-17α, G-CSF, GM-CSF, IFN-γ, IL-1α, IL-1β, IL-12, IL-13, IL-15, IL-9, IL-22, IL-23, IL-35, M-CSF, IL-1Ra, and a combination thereof. In a particular aspect, the plasma biomarker or cell surface maker comprises TNF-α, IL-17α, MIP-1α, and IL-1Ra.

In some aspects of the disclosure, the change in plasma biomarker or cell surface marker is a decrease in the level at least one plasma biomarker or cell surface marker. In some aspects, the plasma biomarker or cell surface marker that exhibits a decrease in the level comprises at least one of the markers selected from IL-10, IL-4, IL-3, IL-5, IL-13, IL-2, LIF, TNF-α, CA-125, and a combination thereof.

In a particular embodiment, the growth or size of the tumor in a subject is measured by MRI. In another embodiment, the growth or size of the tumor is measured by CT scan. In other embodiments, the tumor in the subject is a recurrent tumor that arose during treatment with the vector. In yet other embodiments, the tumor in the subject is a metastatic tumor that arose during treatment with the vector.

In one aspect, the methods of the disclosure further comprise administration of an effective amount one or more chemotherapeutic agents. One or more chemotherapeutic agents that can be administered using the methods of the present disclosure include, but are not limited to, Acivicin; Aclarubicin; Acodazole Hydrochloride; Acronine; Adriamycin; Adozelesin; Aldesleukin; Alimta; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bevacizumab, Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; pazotinib; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sorafinib; Sparfosate Sodium; Sparsomycin; Spirogermanium Hydrochloride; Spiromustine; Spiroplatin; Streptonigrin; Streptozocin; Sulofenur; Sunitinib; Talisomycin; Taxol; Tecogalan Sodium; Tegafur; Teloxantrone Hydrochloride; Temoporfin; Teniposide; Teroxirone; Testolactone; Thiamiprine; Thioguanine; Thiotepa; Tiazofuirin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; or Zorubicin Hydrochloride. Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc.

In some aspects of the disclosure, the one or more chemotherapeutic agents are selected from the group consisting of altretamine, raltritrexed, topotecan, paclitaxel, docetaxel, cisplatin, carboplatin, oxaliplatin, liposomal doxorubicin, gemcitabine, cyclophosphamide, vinorelbine, ifosfamide, etoposide, altretamine, capecitabine, irinotecan, melphalan, pemetrexed, bevacizumab, and albumin bound paclitaxel. In a particular aspect, the chemotherapeutic agent is paclitaxel.

In certain embodiments, identifying a candidate for the vector therapy or combination therapy comprises measuring various characteristics of tumor angiogenesis, for example, reduction in size of the tumor, inhibition of tumor growth, reduction in angiogenesis, reduction in neo-vascularization, or any known characteristics of angiogenesis. In other embodiments, identifying a candidate for the vector therapy or combination therapy comprises measuring plasma biomarkers or cell surface biomarkers. In some embodiments, the plasma biomarkers or cell surface biomarkers are associated with angiogenesis or fever. In certain embodiments, the biomarkers are selected from the group consisting of C-reactive protein (CRP), protein C, interleukin (IL)-6, IL-8, IL-10, IL-10, TNF-α, sTNFRI, sTNFRII, monocyte chemotactic protein-1, ICAM-1, VEGF, FGF, and E-selectin. In other aspects, the plasma biomarker or cell surface biomarker is selected from the group consisting of MCP-1, MIP-1, MIP-2, MIP-1α, MIP-1β, MIG, RANTES, IP-10, IL-1α, IL-1β, IL-2, IL-3, IL-4, IL-5, IL-6, IL-8, IL-9, IL-10, IL-12, IL-13, IL-15, IL-17α, IL-22, IL-23, IL-35, LIF, TNF-α, TNF-β, TGF-β1, VEGF, G-CSF, IFN-α, IFN-β, IFN-γ, M-CSF, IL-1Ra, eotaxin, CA-125, and a combination thereof.

In certain aspects, once the subject or the subject population is identified as a candidate, the one or more chemotherapeutic agents are administered prior to administering the vector, concomitantly with administration of a vector, or after administration of a vector. In other aspects, after a subject or a subject population is identified as a candidate, the vector is administered prior to the one or more chemotherapeutic agents for at least one day earlier, at least two days earlier, at least three days earlier, at least four days earlier, at least five days earlier, at least six days earlier, at least seven days earlier, at least nine days earlier, at least 10 days earlier, at least two weeks earlier, at least three weeks earlier, at least four weeks earlier, at least one month earlier, at least two months earlier, or more.

In one embodiment of the present disclosure, the disclosure includes a method of stabilizing a disease or disorder associated with cancer. In some embodiments, the disclosure includes a method of stabilizing a disease or disorder associated with metastatic colorectal cancer (mCRC), advanced nonsquamous non-small cell lung cancer (NSCLC), metastatic renal cell carcinoma (mRCC), glioblastoma multiforme (GBM), Müllerian cancer, ovarian cancer, peritoneal cancer, fallopian tube cancer, or uterine papillary serousspects, the present disclosure reduces the volume of malignant peritoneal fluid, e.g., ascites, reduces pain to the subject, prolongs survival of the subject, or any combinations thereof. The tumor that can be reduced, inhibited, or treated with the combination of the vector and the one or more chemotherapeutic agents can be a solid tumor, a primary tumor, or a metastatic tumor. The term “metastatic” or “metastasis” refers to tumor cells that are able to establish secondary tumor lesions in another parts or organ.

A “solid tumor” includes, but is not limited to, sarcoma, melanoma, carcinoma, or other solid tumor cancer. “Sarcoma” refers to a tumor which is made up of a substance like the embryonic connective tissue and is generally composed of closely packed cells embedded in a fibrillar or homogeneous substance. Sarcomas include, but are not limited to, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” refers to a tumor arising from the melanocytic system of the skin and other organs. Melanomas include, for example, acra-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, metastatic melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidernoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma viflosum.

Additional cancers that can be inhibited or treated include, for example, Leukemia, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, papillary thyroid cancer, neuroblastoma, neuroendocrine cancer, esophageal cancer, genitourinary tract cancer, malignant hypercalcemia, cervical cancer, endometrial cancer, adrenal cortical cancer, prostate cancer, Müllerian cancer, ovarian cancer, peritoneal cancer, fallopian tube cancer, or uterine papillary serous carcinoma.

In other embodiments, the subject has had up to three, up to two, or up to one previous line of chemotherapy. In some aspects, the previous line of chemotherapy is a platinum-based chemotherapy (e.g., cisplatin, oxaliplatin, or carboplatin). In yet other embodiments, the subject has not had more than 3 prior lines of chemotherapy for recurrent cancer.

The priming dose or doses and the therapeutically effective dose or doses of the vector administered as part of the present disclosure can be measured in virus particles (VPs). In one embodiment, the priming dose is identical to the therapeutically effective dose. In another embodiment, the priming dose is lower than the therapeutically effective dose. In another embodiment, the priming dose is higher than the therapeutically effective dose. In one aspect, the priming dose is administered more than once. In another aspect, the priming dose is repeatedly administered. In one aspect, the therapeutically effective dose is administered more than once. In another aspect, the therapeutically effective dose is repeatedly administered.

In some embodiments, the priming dose of the vector is administered at an amount of less than about 1×10¹⁵, less than about 1×10¹⁴, less than about 5×10¹³, less than about 4×10¹³, less than about 3×10¹³, less than about 2×10¹³, less than about 1×10¹³, less than about 9×10¹², less than about 8×10¹², less than about 7×10¹², less than about 6×10¹², less than about 5×10¹², less than about 4×10¹², less than about 3×10¹², less than about 2×10¹², less than about 1×10¹², less than about 9×10¹¹, less than about 8×10¹¹, less than about 7×10¹¹, less than about 6×10¹¹, less than about 5×10¹¹, less than about 4×10¹¹, less than about 3×10¹¹, less than about 2×10¹¹, less than about 1×10¹¹, less than about 9×10¹⁰, less than about 8×10¹⁰, less than about 7×10¹⁰, less than about 6×10¹⁰, less than about 5×10¹⁰, less than about 4×10¹⁰, less than about 3×10¹⁰, less than about 2×10¹⁰, or less than about 1×10¹⁰ virus particles.

In other embodiments, the therapeutically effective dose of the vector is administered at an amount of at least about 1×10¹⁵, at least about 1×10¹⁴, at least about 5×10¹³, at least about 4×10¹³, at least about 3×10¹³, at least about 2×10¹³, at least about 1×10¹³, at least about 9×10¹², at least about 8×10¹², at least about 7×10¹², at least about 6×10¹², at least about 5×10¹², at least about 4×10¹², at least about 3×10¹², at least about 2×10¹², at least about 1×10¹², at least about 9×10¹¹, at least about 8×10¹¹, at least about 7×10¹¹, at least about 6×10¹¹, at least about 5×10¹¹, at least about 4×10¹¹, at least about 3×10¹¹, at least about 2×10¹¹, at least about 1×10¹¹, at least about 9×10¹⁰, at least about 8×10¹⁰, at least about 7×10¹⁰, at least about 6×10¹⁰, at least about 5×10¹⁰, at least about 4×10¹⁰, at least about 3×10¹⁰, at least about 2×10¹⁰, or at least about 1×10¹⁰ virus particles.

In one embodiment, the at least one priming dose and the at least one therapeutically effective dose of the vector in the combination therapy with the one or more chemotherapeutic agents are lower than the at least one priming dose and the at least one therapeutically effective dose used for the therapy without the one or more chemotherapeutic agents (e.g., a monotherapy therapy using the vector alone). For example, a therapeutically effective dose of the vector in the combination therapy with paclitaxel includes, but is not limited to equal to or less than about 1×10¹³, 9×10¹², 8×10¹², 7×10¹², 6×10¹², 5×10¹², 4×10¹², 3×10¹², 2×10¹², 1×10¹², 9×10¹¹, 8×10¹¹, 7×10¹¹, 6×10¹¹, 5×10¹¹, 4×10¹¹, 3×10¹¹, 2×10¹¹, 1×10¹¹, 9×10¹⁰, 8×10¹⁰, 7×10¹⁰, 6×10¹⁰, 5×10¹⁰, 4×10¹⁰, 3×10¹⁰, 2×10¹⁰, or 1×10¹⁰ virus particles. And, a priming dose of the vector in the combination therapy with paclitaxel includes, but is not limited to equal to or less than about 1×10¹³, 9×10¹², 8×10¹², 7×10¹², 6×10¹², 5×10¹², 4×10¹², 3×10¹², 2×10¹², 1×10¹², 9×10¹¹, 8×10¹¹, 7×10¹¹, 6×10¹¹, 5×10¹¹, 4×10¹¹, 3×10¹¹, 2×10¹¹, 1×10¹¹, 9×10¹⁰, 8×10¹⁰, 7×10¹⁰, 6×10¹⁰, 5×10¹⁰, 4×10¹⁰, 3×10¹⁰, 2×10¹⁰, or 1×10¹⁰ virus particles.

In one embodiment, the priming dose of the vector is administered at an amount of at least about 1×10¹¹ virus particles. In another embodiment, the priming dose of the vector is administered at an amount of at least about 1×10¹² virus particles. In another embodiment, the priming dose of the vector is administered at an amount of at least about 1×10¹³ virus particles. In another embodiment, the priming dose of the vector is administered at an amount of at least about 1×10¹⁴ virus particles. In other embodiments, the priming dose of the vector is administered at an amount of at least about 1×10⁷, 1×10⁸, 1×10⁹, 1×10¹⁰, or 5×10¹⁰ virus particles.

In another embodiment, the therapeutically effective dose of the vector is administered at an amount of at least about 1×10¹¹ virus particles. In another embodiment, the therapeutically effective dose of the vector is administered at an amount of at least about 1×10¹² virus particles. In another embodiment, the therapeutically effective dose of the vector is administered at an amount of at least about 1×10¹³ virus particles. In another embodiment, the therapeutically effective dose of the vector is administered at an amount of at least about 1×10¹⁴ virus particles. In other embodiments, the therapeutically effective dose of the vector is administered at an amount of at least about 1×10⁷, 1×10⁸, 1×10⁹, 1×10¹⁰, or 5×10¹⁰ virus particles.

The dose of paclitaxel can be measured in mg per square meter of body-surface area (mg/m²). In one aspect, the dose of paclitaxel in the combination therapy with the vector is lower than the dose of paclitaxel without the vector (e.g., a therapy using paclitaxel alone). Non-limiting examples of an effective amount of paclitaxel include at least about 10 mg/m², at least about 20 mg/m², at least about 30 mg/m², at least about 40 mg/m², at least about 50 mg/m², at least about 60 mg/m², at least about 70 mg/m², at least about 80 mg/m², at least about 90 mg/m², at least about 100 mg/m², at least about 110 mg/m², at least about 120 mg/m², at least about 130 mg/m², at least about 140 mg/m², at least about 150 mg/m², at least about 160 mg/m², at least about 170 mg/m², at least about 180 mg/m², at least about 190 mg/m², or at least about 200 mg/m². In one aspect, the effective amount of paclitaxel is about 175 mg/m². In another aspect, the effective amount of paclitaxel is about 135 mg/m².

In some aspects, the effective amount of paclitaxel includes about 10 mg/m² to about 100 mg/m², about 20 mg/m² to about 90 mg/m², about 30 mg/m² to about 90 mg/m²; about 40 mg/m² to about 90 mg/m²; about 50 mg/m² to about 90 mg/m², about 60 mg/m² to about 90 mg/m², or about 70 mg/m² to about 90 mg/m². In a particular aspect, the effective amount of paclitaxel is about 80 mg/m².

In some aspects, the paclitaxel is infused for at least 10 minutes, at least 20 minutes, at least 30 minutes, at least 40 minutes, at least 50 minutes, at least 60 minutes, at least 70 minutes, at least 80 minutes, at least 90 minutes, at least 100 minutes, at least 110 minutes, at least 120 minutes, at least 150 minutes, at least 180 minutes, at least 210 minutes, at least 240 minutes, at least 270 minutes, or at least 300 minutes. In some aspects, the paclitaxel is infused for at least 4 hours, at least 5 hours, at least 6 hours, at least 7 hours, at least 8 hours, at least 9 hours, at least 10 hours, or at least 12 hours. In some aspects, the pactlitaxel is infused for at least 24 hours. In one aspect, the paclitaxel is infused for at least one hour. In another aspect, the pactlitaxel is infused for at least 3 hours. The infusion methods for paclitaxel can be used any methods known in the art. For example, paclitaxel can be administered through an in-line filter with a microporous membrane not greater than 0.22 microns over three hours.

In a specific embodiment, the priming dose of the vector is administered at an amount of 3×10¹² to 1×10¹³ VPs, the therapeutically effective dose of the vector is administered at an amount of 3×10¹² to 1×10¹³ VPs, and paclitaxel is administered at an effective amount of about 70 mg/m² to about 90 mg/m².

In some aspects of the disclosure, the subject is premedicated with one or more additional agents before being administered the priming dose of the vector. In some aspects, the subject is premedicated with one or more additional agents before being administered the therapeutic dose of the vector. In some aspects, the subject is premedicated with one or more agents before being administered paclitaxel. In some aspects, the one or more additional agent is a corticosteroid (e.g., dexamethasone). In some aspects, the one or more additional agent is diphenhydramine. In some aspects, the one or more additional agent is an H2 antagonist (e.g., cimetidine or ranitidine). In some aspects the one or more additional agents comprise corticosteroids (such as dexamethasone), diphenhydramine and H2 antagonists (such as cimetidine or ranitidine).

The present disclosure provides methods of treating a tumor in a subject in need thereof comprising administering at least one priming dose of the vector, at least one therapeutically effective dose of the vector, and one or more chemotherapeutic agents. The regimen used for administering the vector and the one or more chemotherapeutic agents comprises repeated administration of the vector and the one or more chemotherapeutic agents. In one embodiment, the vector is repeatedly administered every day, once in about 2 days, once in about 3 days, once in about 4 days, once in about 5 days, once in about 6 days, once in about 7 days, once in about 2 weeks, once in about 3 weeks, once in about 4 weeks, once in about 5 weeks, once in about 6 weeks, once in about 7 weeks, once in about 2 months, or once in about 6 months. In another embodiment the one or more chemotherapeutic agents are repeatedly administered every day, every two days, every three days, every four days, every five days, every six days, every seven days, every eight days, every nine days, or every ten days. In some aspects, the one or more chemotherapeutic agents are administered once in about 7 days, once in about 2 weeks, once in about 3 weeks, once in about 4 weeks, once in about 2 months, once in about 3 months, once in about 4 months, once in about 5 months, or once in about 6 months. In a particular embodiment, the vector is administered every 2 months and paclitaxel is administered every week.

### V. Pharmaceutical Compositions

Also provided in the disclosure is a pharmaceutical composition comprising a vector expressing a FAS-chimera protein used in the methods of the disclosure. The pharmaceutical composition can be formulated for administration to mammals, including humans. The pharmaceutical compositions used in the methods of this disclosure comprise pharmaceutically acceptable carriers, including, e.g., ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. In one embodiment, the composition is formulated by adding saline.

The compositions of the present disclosure can be administered by any suitable method, e.g., parenterally (e.g., includes subcutaneous, intravenous, intramuscular, intra-articular, intra-synovial, intrasternal, intrathecal, intrahepatic, intralesional and intracranial injection or infusion techniques), intraventricularly, orally, by inhalation spray, topically, rectally, nasally, buccally, vaginally or via an implanted reservoir. As described previously, the composition comprising a nucleic acid construct which comprises a FAS-chimera gene in an endothelial cell and thereby induces apoptosis of the endothelial cell. Accordingly, the composition can inhibit, reduce, or decrease the size of a tumor or a metastasis thereof by inhibiting neo-vascularization and/or angiogenesis of the tumor endothelial cells. Likewise, the VEGF antagonist used in combination with the nucleic acid construct inhibit neo-vascularization and/or angiogenesis through direct inhibition of VEGF activity. Therefore, in one embodiment, the combination therapy is delivered systemically or locally. For systemic or local delivery, the pharmaceutical formulation containing the nucleic acid construct, the adenovirus, or the homogeneous population of the adenovirus can utilize a mechanical device such as a needle, cannula or surgical instruments.

Sterile injectable forms of the compositions used in the methods of this disclosure can be aqueous or oleaginous suspension. These suspensions can be formulated according to techniques known in the art using suitable dispersing or wetting agents and suspending agents. The sterile, injectable preparation can also be a sterile, injectable solution or suspension in a non-toxic parenterally acceptable diluent or solvent, for example as a suspension in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions can also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms can also be used for the purposes of formulation.

Parenteral formulations can be a single bolus dose, an infusion or a loading bolus dose followed with a maintenance dose. These compositions can be administered at specific fixed or variable intervals, e.g., once a day, or on an “as needed” basis.

Certain pharmaceutical compositions used in the methods of this disclosure can be orally administered in an acceptable dosage form including, e.g., capsules, tablets, aqueous suspensions or solutions. Certain pharmaceutical compositions also can be administered by nasal aerosol or inhalation. Such compositions can be prepared as solutions in saline, employing benzyl alcohol or other suitable preservatives, absorption promoters to enhance bioavailability, and/or other conventional solubilizing or dispersing agents.

An effective amount of the chemotherapeutic agent is available in the art. In one aspect, for example, an effective amount of bevacizumab can be at least about 1 mg/kg, 2 mg/kg, 3 mg/kg, 4 mg/kg, or 5 mg/kg.

### EXAMPLES

### Example 1

### Construction and Cloning of the Viral Vector

The vector was constructed using a backbone containing most of the genome of adenovirus type 5, as well as partial homology to an adaptor plasmid, which enables recombination.

The E1 early transcriptional unit was deleted from the backbone plasmid, and further modified by deleting the pWE25 and the Amp resistance selection marker site.

The adaptor plasmid contains sequences of the Ad5, CMV promoter, MCS, and SV40 polyA. The adaptor plasmid was modified to delete the CMV promoter, and the PPE-1 promoter and Fas-c fragment were inserted by restriction digestion. The modified PPE-1 promoter (PPE-1-3X, SEQ ID NO: 18) and the Fas-chimera transgene (Fas-c, SEQ ID NO: 9) were utilized for construction of the adenoviral vector. The PPE-1-(3X)-Fas-c element (2115 bp) was constructed from the PPE-1-(3X)-luc element. This element contains the 1.4 kb of the murine preproendothelin PPE-1-(3X) promoter, the Luciferase gene, the SV40 polyA site and the first intron of the murine ET-1 gene, originated from the pEL8 plasmid (8848 bp) used by Harats et al (Harats D. et al., JCI, 1995). The PPE-3-Luc cassette was extracted from the pEL8 plasmid using the BamHI restriction enzyme. The Luciferase gene was substituted by the Fas-c gene [composed of the extra cellular and intra membranal domains of the human TNF-R1 (Tumor Necrosis Factor Receptor 1, SEQ ID NO: 4) and of the Fas (p55) intracellular domain (SEQ ID NO: 8) (Boldin et al, JBC, 1995)] to obtain the PPE-1-3x-Fas-c cassette.

PPE-1 (3x)-Fas-c Plasmid—The cassette was further introduced into the backbone plasmid by restriction digestion, resulting with the PPE-1 (3x)-Fas-c plasmid.

Adaptor-PPE-1(3x)-Fas-c Plasmid—The PPE-1-3x-Fas-c element was extracted from the first generation construct PPE-1-3x-Fas-c plasmid, and was amplified with designated PCR primers introducing SnaB1 and EcoR1 restriction sites at the 5′-and-3′-end respectively. These sites were used to clone the PPE-Fas-c fragment into the adaptor plasmid digested with SnaB1 and EcoR1, resulting in the adaptor-PPE-1-3x-Fas-c used for transfection of the host cells (for example, PER.C6 cells).

### Example 2

Phase 2 Study of Gene Therapy with VB-111 in Recurrent Glioblastoma with Dual Mechanism of Angiogenic Endothelial-Specific Death Receptor and Antitumor-Specific Immune Induction

OBJECTIVES: The objectives of this Phase 1/2 study were to evaluate the safety, tolerability, and efficacy of single and multiple doses of VB-111 (1×10¹², 3×10¹², and 1×10¹³ viral particles) in patients with recurrent GBM (rGBM), the distribution of VB-111, and the level of antibodies to the adenovirus vector. The study was also extended to evaluate the safety, tolerability, and efficacy of combination treatment with multiple doses of VB-111 (3×10¹² or 1×10¹³ VP) together with bevacizumab in patients with rGBM. Overall survival (OS) was the primary efficacy endpoint.

### Methods

Study Design: This study was a prospective, open-label, dose-escalating, Phase 1/2 study of VB-111 conducted at 3 centers in the US and 1 center in Israel (University of Texas Health Science Center, San Antonio, Tex.; Dana Farber Cancer Center, Boston, Mass.; Duke University Medical Center, Durham, N.C.; and Tel Aviv Medical Center, Tel Aviv, Israel; clinicaltrials.gov identifier NCT01260506). The study was conducted in accordance with the Declaration of Helsinki and the International Conference on Harmonization (ICH) Guidelines for Good Clinical Practice, including collection of written informed consent from all patients prior to performing any study-related procedures. The study protocol and patient materials were approved by the relevant regulatory agencies and site-specific ethics committees.

Patient Selection: Patients eligible for this study were 18 years or older with a histologically confirmed diagnosis of glioblastoma multiforme. Patients were required to have measurable disease according to the Response Assessment in Neuro-Oncology (RANO) criteria¹⁶ and disease progression or recurrence following standard of care treatment with temozolomide and radiation; any tumor sizes, including unresectable tumors, were allowed. For the dose-escalation portion of the study, subjects were excluded if imaging showed major mass effect of the tumor (defined as <5 mm shift or evidence of herniation). For all cohorts, patients were excluded for receipt of prior antiangiogenic therapy or stereotactic radiation. Other key eligibility criteria included a Karnofsky performance status of at least 70% and adequate hematologic, renal, and hepatic function. Exclusion criteria included recently active cardiovascular disease, recent surgery, proliferative retinopathy, liver disease, use of an investigative agent (within 4 weeks), or an uncontrolled comorbidity.

Treatments administered and dose-escalation scheme: VB-111 was manufactured in a current Good Manufacturing Practice facility. Vials were diluted with normal saline for infusion and administered as a single IV infusion. The starting dose was 1×10¹² VP, which represents a two-dose level reduction from the maximum evaluated safe dose of 1×10¹³ VP determined in a previous Phase 1 study.

Dose escalation to planned dose levels of 3×10¹² VP and 1×10¹³ VP followed a standard 3+3 design. If 1 patient experienced a dose-limiting toxicity (DLT; defined as any treatment-related, grade 3 toxicity), the cohort was to be expanded to 6 patients. If 0 of 3 patients in the expanded cohort experienced a DLT, escalation proceeded to the subsequent dose level. The maximum tolerated dose (MTD) was defined as the highest dose at which fewer than 33% patients experienced a DLT up to the maximum planned dose of 1×10¹³ VP. Toxicity was graded according to the National Cancer Institute Common Terminology Criteria for Adverse Events (Version 4.0).

Dosing cohorts: The sub-therapeutic (SubT) cohort includes patients in the dose escalation (DE) who received doses below 1×10¹³ VP. As interpatient dose escalation was permitted, patients from the SubT cohort had an opportunity to later receive 1×10¹³ VP of VB 111.

The limited exposure (LE) cohort includes patients who received multiple VB-111 doses at the maximum planned dose (1×10¹³ VP) in 56 day intervals.

The LE-DE cohort includes all patients who received at least 1 dose of 1×10¹³ VP (without bevacizumab); this cohort was evaluated for safety only.

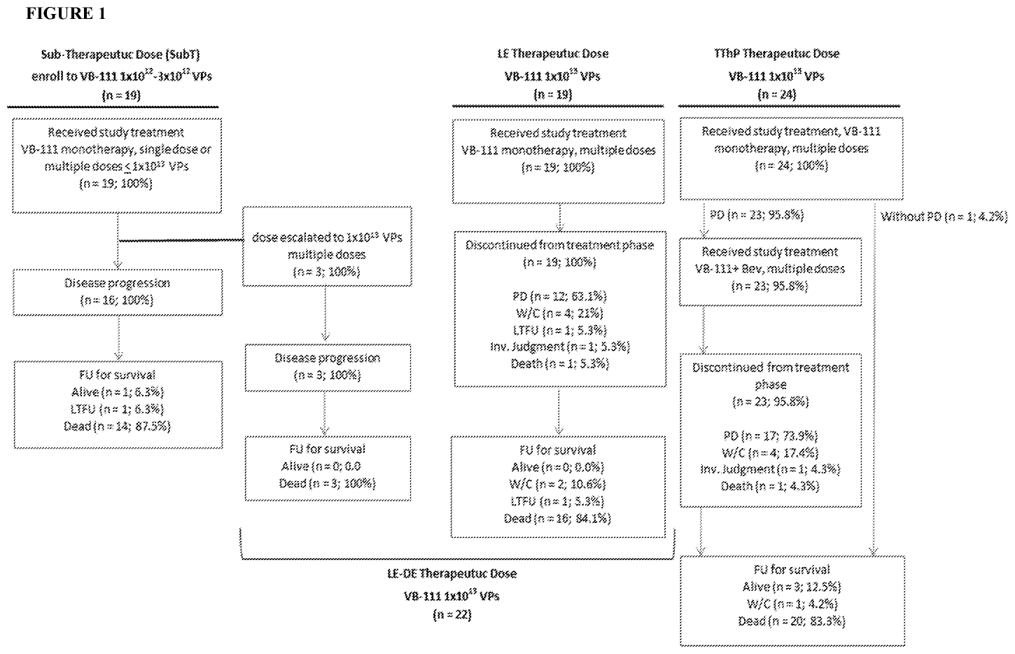

The treatment through progression (TThP) cohort was added by protocol amendment to allow patients to receive VB-111 (1×10¹³ VP) every 56 days until progression (TThP cohort, monotherapy phase); beyond further progression, these patients received VB-111 (1×1013 VP) every 56 days in combination with salvage bevacizumab (10 mg/kg IV) every 2 weeks (TThP cohort, combination phase) (FIG. 1).

Concomitant medications: To avoid fever following study drug administration, patients received acetaminophen (1 g) before dosing and subsequently as needed for 72 hours after dosing. Because of a concern for vascular disrupting effects of the study agent and possible cerebral edema, all patients received dexamethasone (4 mg orally twice daily) for 14 days with the first VB-111 infusion and for 3 days with subsequent infusions.

Biodistribution analysis: A quantitative polymerase chain reaction (qPCR) method was implemented to detect adenovirus vector VB-111 in human whole blood and urine samples. Isolation of DNA was performed using a Qiagen DNeasy Blood and Tissue Extraction Kit (Qiagen, Inc., Germantown, Md.). Five microliters of this eluate was then analyzed by a validated qPCR assay for the presence of the adenovirus hexon gene using the Applied Biosystems 7900HT (Thermo Fisher Scientific, Waltham, Mass.). Each sample was analyzed in triplicate, with the third replicate spiked with 100 copies of VB-111 DNA to determine whether any PCR inhibitors were present in the sample. Finally, the resulting mean copy number from replicates was converted to copies per microgram of DNA.

Response evaluation: Patients were followed with magnetic resonance imaging (MRI) scans performed at the end of every other cycle. The local investigator assessed response and disease progression using RANO criteria, and central review of response was performed post hoc (BIOCLINICA®, Princeton, N.J.).

### Statistical Analysis

Comparisons between subgroups of the Phase II study: Demographic variables and relevant clinical characteristics were described and compared among the 3 groups (SubT, LE and TThp), using the Kruskal-Wallis Test in the case of continuous variables and Fisher's Exact Test in the case of categorical variables.

Overall survival (time to death in days) was assessed using Kaplan-Meier curves for each group separately and overall. The log-rank test was used to test differences in survival among groups.

Progression at 6 months after the start of therapy and progression-free survival (PFS) was examined for all groups and was based on RANO assessments. Two sets of progression data were analyzed: (i) data originating from the clinical sites and reviewed by neuro-oncologists, and (ii) data from an external vendor (Bioclinica Medical Imaging) and reviewed by neuro-oncologists.

For TThp patients, 2 progression endpoints were defined: progression after high-dose monotherapy and progression after combination VB-111+bevacizumab therapy. Progression-free time for these endpoints was measured from the start of monotherapy in the first case and from start of combination therapy in the second. One patient in the TThp group did not experience progression and remained on monotherapy throughout follow-up for 44 months, and is not included in progression results. The log-rank test was used to test differences in PFS among groups.

The slope of log tumor measurements over time was calculated for each patient, and the slopes were compared across therapy groups using the Wilcoxon Rank Sum test.

Comparisons between the TThp cohort and a historical cohort: A historical control group was established based on a meta-analysis of trials and case series that included rGBM patients treated with bevacizumab.

In order to construct a historical control group for the Phase 1/2 single-arm study of VB-111, a Medline search was conducted by two researchers to identify publications of any study or case series of patients with rGBM who were treated with bevacizumab monotherapy, with keywords of “rGBM,” “recurrent glioblastoma,” “bevacizumab,” and “avastin.” Publications were reviewed and a consensus was reached regarding eligibility in the meta-analysis based on the following pre-specified inclusion criteria:
Prospective or retrospective design
Published as a manuscript in 2005 or later
Included at least 20 patients in the bevacizumab arm who were:
   Adults aged 18 years or older
   Diagnosed with rGBM (first or subsequent recurrence)
   Treated with any prior regimen, excluding prior bevacizumab
   Treated with bevacizumab monotherapy at any dose (note: for studies with several treatment arms, the data for the bevacizumab group may have been separated out)
Reported detailed survival outcomes for individual patients (either tabulated or as a Kaplan-Meier curve)

For each eligible publication, the following data were extracted:
Citation
Number of patients treated (total and in the bevacizumab arm)
Individual and summary patient data:
   Number of recurrences
   Age (median, average)
   Sex
   Prior therapy (number and type, eg, drug/radiation/surgery)
   Time since prior radiotherapy
   Surgery at the time of recurrence, percent full resection
   Initial resection: full, partial, or biopsy
   Time from diagnosis to recurrence
   MGMT methylation status, EGFR-V3, IDH-1, and other genetic status
   Karnofsky performance status
   Bevacizumab dose received
   Diameter of maximum enhancing tumor at baseline
   Corticosteroid use
   Post-study therapy
Median PFS and OS

Survival data were extracted either from tables of individual patient data or from Kaplan-Meier curves. In the latter case, data were extracted using DigitizeIt (Bormann, Braunschweig, Germany), a program that converts graphs into numeric data.

The search results yielded 8 publications that were eligible for the meta-analysis, which included 694 rGBM patients treated with bevacizumab monotherapy: Friedman H S et al., “Bevacizumab alone and in combination with irinotecan in recurrent glioblastoma,” J Clin Oncol 27:4733-4740 (2009); Duerinck J et al., “Patient outcome in the Belgian medical need program on bevacizumab for recurrent glioblastoma,” J Neurol, 262:742-751 (2015); Taal W et al., “Single-agent bevacizumab or lomustine versus a combination of bevacizumab plus lomustine in patients with recurrent glioblastoma (BELOB trial): a randomised controlled phase 2 trial,” Lancet Oncol 15:943-953 (2014); Kreisl T N et al., “Phase II trial of single-agent bevacizumab followed by bevacizumab plus irinotecan at tumor progression in recurrent glioblastoma,” J Clin Oncol 27:740-745 (2009); Chamberlain M C, Johnston S K, “Salvage therapy with single agent bevacizumab for recurrent glioblastoma,” J Neurooncol 96:259-269 (2010); Field K M et al, “Randomized phase 2 study of carboplatin and bevacizumab in recurrent glioblastoma,” Neuro Oncol 17:1504-1513 (2015); Nagane M et al., “Phase II study of single-agent bevacizumab in Japanese patients with recurrent malignant glioma,” Jpn J Clin Oncol 42:887-895 (2012); and Chen C et al., “Clinical outcomes with bevacizumab-containing and non-bevacizumab-containing regimens in patients with recurrent glioblastoma from US community practices,” J Neurooncol 122:595-605 (2015). These studies are shown below in Table 5.

TABLE 5
Studies ion the meta-analysis of rGBM patients treated with bevacizumab monotherapy.

| | | | | Median | >first | | Median |
|---|---|---|---|---|---|---|---|
| | | | | age | recurrence | | survival |
| Study | Design | Year | N | (years) | (%) | Performance status | (weeks) |
| BELOB trial | Phase 2 RCT | 2014 | 50 | 58 | 0 | ECOG (patients) | 34.8 |
| (Taal et al.) | | | | | | 0 (13); 1(32); 2(5) | |
| BRAIN trial | Phase 2 RCT | 2009 | 85 | 54 | 19 | KPS (patients) | 40.5 |
| (Friedman et al.) | | | | | | 90-100 (38); 10-80 (47) | |
| Kreisl et al. | Phase 2 RCT | 2009 | 48 | 53 | N/A | KPS median (range) | 31.0 |
| | | | | | | 90 (60-100) | |
| Chamberlain et al. | Retrospective | 2010 | 50 | 64 | 68 | KPS median (range) | 37.0 |
| | | | | | | 80 (60-100) | |
| Field et al. | Phase 2 RCT | 2015 | 62 | 55 | 31 | KPS (patients) | 32.6 |
| | | | | | | 90-100 (22); 70-80 | |
| | | | | | | (28), <70 (10); NA (2) | |
| Nagane et al. | Phase 2 | 2012 | 29 | 57 | 42 | KPS (patients) | 45.7 |
| | single arm | | | | | 90-100 (17); 70-80 (12) | |
| Chen et al. | Retrospective | 2015 | 57 | 61 | 0 | KPS (patients) | 29.4 |
| | | | | | | 90-100 (13); 70-80 | |
| | | | | | | (10); ≤70 (20); NA (14) | |
| Duerinck et al. | Prospective | 2015 | 313 | 55 | 88 | ECOG (patients) | 26.0 |
| | cohort | | | | | 0 (30); 1 (204); 2 (57); | |
| | | | | | | 3 (12); NA (10) | |
| Pooled historical | | | 694 | | | | 32.1 |
| cohort | | | | | | | |
| VB111 TThp | Phase 2 | NA | 24 | 60 | 50 | KPS median (range) | 59.1 |
| cohort | single arm | | | | | 80 (60-100) | |

Abbreviations:
ECOG, Eastern Cooperative Oncology Group;
KPS, Karnofsky performance status;
N, number of patients treated;
NA = not applicable;
RCT, randomized controlled trial;
TThP, treatment through progression

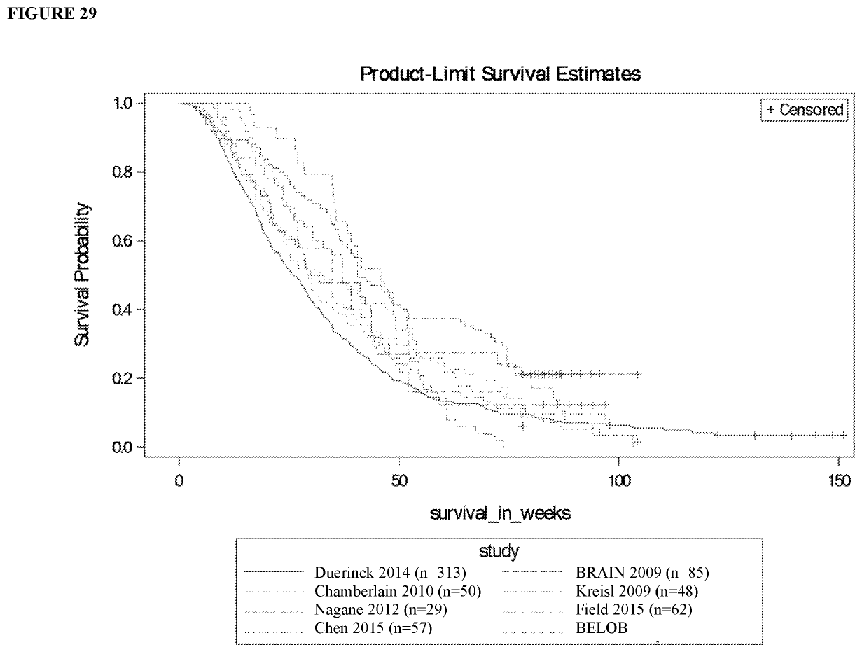

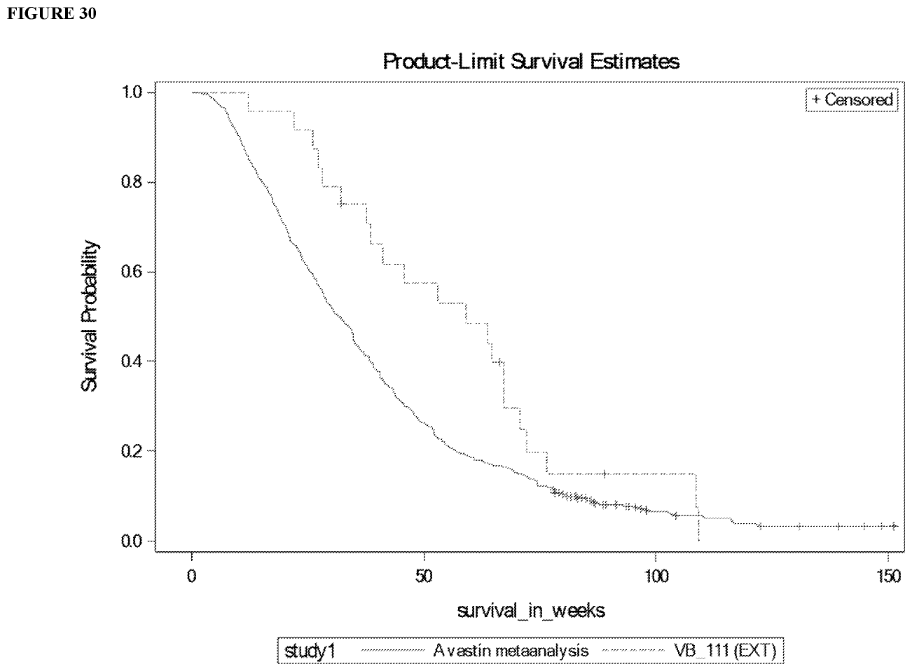

Data across all studies were pooled to form a historical control group, with survival compared with the TThp cohort of the VB-111 study using a log-rank test. Kaplan-Meier curves were re-constructed from the numeric data for each of the 8 studies, as provided in FIG. 29 for individual studies and in FIG. 30 for the pooled bevacizumab cohort (plotted with the TThp cohort of the VB-111 study). The log-rank chi-square statistic was 4.74 on 1 degree of freedom (P=0.0294). From a Cox regression model, the estimated hazard ratio of the VB-111 group compared to the pooled bevacizumab cohort was 0.62 (95% CI 0.40-0.96).

A heterogeneity analysis indicated that the largest study in the historical control group (Duerinck et al 2015) had much poorer survival than that of the other studies, which may be attributable to the type of data presented (a report of a treatment program rather than a clinical research study), as it is often noted that patients entering clinical research studies have a generally better prognosis. A Cox regression of all 8 studies indeed confirms significant heterogeneity of results, with a Wald chi-square of 26.96 on 7 degrees of freedom (p=0.0003). Most of this heterogeneity was accounted for by the difference between the results of the Duerinck study and those of the Friedman and the Nagane studies.

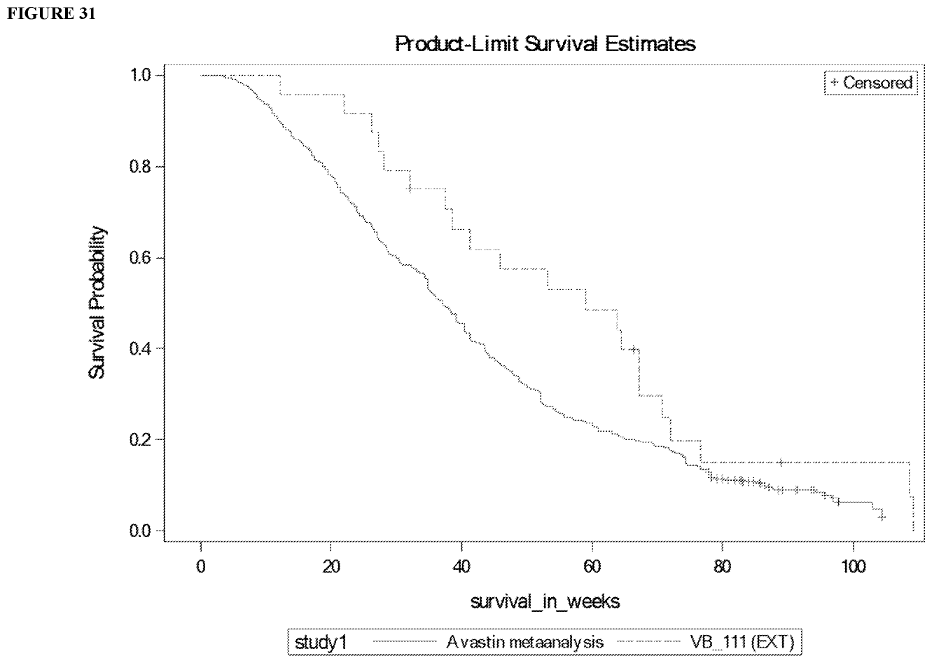

In a sensitivity analysis, the Duerinck study was removed from the pooled bevacizumab cohort and the comparison with the TThp cohort was repeated to determine whether the significant survival difference identified between the VB-111 TThp cohort and the pooled bevacizumab cohort was robust to the exclusion of the Duerinck study (FIG. 31). The log-rank chi-square statistic decreased to 4.29 on 1 degree of freedom but was still statistically significant (p=0.0382). From a Cox regression model, the estimated hazard ratio of the VB-111 TThP cohort compared to the pooled bevacizumab cohort did not change, remaining at 0.62, but its CI was slightly wider (95% CI 0.39-0.98).

### Results

Sixty-two (62) patients with rGBM were enrolled into 3 consecutive cohorts: SubT (n=19), LE (n=19), and TThP (n=24). Three patients were initially started on lower doses in the SubT cohort and underwent interpatient dose escalation to 1×10¹³ VP per protocol; thus, 22 (19+3) patients are included in the LE-DE cohort for safety evaluation. As of the data cutoff date, 3 patients remain alive (all in the TThP cohort), 2 were lost to follow-up, 3 withdrew consent and 53 died (FIG. 1). Patient characteristics are presented in Table 6.

TABLE 6
Baseline Patient Characteristics

| | LE Sub- | | LE-DE | | TThp | | |
|---|---|---|---|---|---|---|---|
| | Therapeutic | | Therapeutic | | Therapeutic | | |
| | Dose | | Dose | | Dose | | p |
| Characteristic | (n = 16) | | (n = 22) | | (n = 24) | | value |
| Median age, years | 56.1 | (28-65) | 55.9 | (27-76) | 60 | (19-72) | 0.77^{a} |
| Sex | | | | | | | 0.49^{b} |
| Male | 11 | (68.8%) | 13 | (59.1%) | 12 | (50.0%) | |
| Female | 5 | (31.3%) | 9 | (40.9%) | 12 | (50.0%) | |
| Race | | | | | | | 0.26^{b} |
| White | 15 | (93.8%) | 22 | (100%) | 24 | (100%) | |
| Asian | 1 | (6.3%) | 0 | (0.0%) | 0 | (0.0%) | |
| Ethnicity | | | | | | | 0.80^{b} |
| Hispanic or Latino | 1 | (6.3%) | 3 | (13.6%) | 4 | (16.7%) | |
| Non-Hispanic or Latino | 15 | (93.8%) | 19 | (86.4%) | 20 | (83.3%) | |
| KPS | | | | | | | 0.10^{b} |
| 90-100 | 12 | (75.0%) | 14 | (63.6%) | 9 | (37.5%) | |
| 70-80 | 4 | (25%) | 7 | (31.8%) | 14 | (58.3%) | |
| ≤60 | 0 | (0.0%) | 1 | (4.5%) | 1 | (4.2%) | |
| Initial surgery | | | | | | | 0.72^{b,c} |
| Biopsy only | 2 | (12.5%) | 3 | (13.6%) | 5 | (20.8%) | |
| Partial resection | 9 | (56.3%) | 11 | (50.0%) | 8 | (33.3%) | |
| Complete resection | 4 | (25%) | 8 | (36.4%) | 9 | (37.5%) | |
| Unknown | 1 | (6.3%) | 0 | (0.0%) | 2 | (8.3%) | |
| Recurrence | | | | | | | 0.22^{b} |
| First | 11 | (68.8%) | 16 | (72.7%) | 13 | (54.2%) | |
| Second | 3 | (18.8%) | 3 | (13.6%) | 10 | (41.7%) | |
| >Second | 2 | (12.5%) | 3 | (13.6%) | 1 | (4.2%) | |
| No. of target lesions | | | | | | | 0.35^{b} |
| 1 | 15 | (93.8%) | 17 | (77.3%) | 22 | (91.7%) | |
| >1 | 1 | (6.3%) | 5 | (22.7%) | 2 | (8.3%) | |
| Tumor volume at enrollment | | | | | | | |
| (cc) | | | | | | | |
| Site measurement: mean, median | 9.9, 5.4 | | 17.5, 12.6 | | 34.1, 11.0 | | 0.56^{a} |
| Core lab measurement: mean, | 17.0, 6.1 | | 20.1, 6.2 | | 33.2, 14.0 | | 0.52^{a} |
| median | | | | | | | |
| Site measurement: % > 32 cc | 6% | | 19% | | 25% | | 0.34^{b} |
| Core lab measurement: % > 32 cc | 19% | | 16% | | 38% | | 0.51^{b} |
| Median days since diagnosis | 302.5 | (215-2593) | 436.5 | (188-6234) | 524.0 | (161-3266) | |
| No. of prior lines of therapy | | | | | | | 0.25^{b} |
| 1 | 12 | (75.0%) | 16 | (72.7%) | 16 | (66.7%) | |
| 2 | 3 | (18.8%) | 3 | (13.6%) | 8 | (33.3%) | |
| >2 | 1 | (6.3%) | 3 | (13.6%) | 0 | (0.0%) | |
| Median (range) | 1 | (1-4) | 1 | (1-4) | 1 | (1-2) | |
| Treatment modality | | | | | | | 0.55^{b} |
| Combined therapy | 14 | (87.5%) | 21 | (95.5%) | 23 | (95.8%) | |
| Sequential therapy | 2 | (12.5%) | 1 | (4.5%) | 1 | (4.2%) | |
| MGMT methylation status | | | | | | | |
| Methylated | 4 | (25.0%) | 4 | (18.2%) | 9 | (37.5%) | 0.14^{bc} |
| Unmethylated | 4 | (25.0%) | 11 | (50.0%) | 5 | (20.8%) | |
| Not done/unknown | 8 | (50.0%) | 7 | (31.8%) | 10 | (41.7%) | |

^{a}Kruskal-Wallis test
^{b}Fisher's exact test
^{c}Omitting the unknown category from the test

Age was similar across cohorts; most patients were previously treated with combined modalities and most had a single lesion at study entry. The TThp cohort had more advanced disease as reflected by larger proportions of patients at second or subsequent relapse, with greater than 1 prior line of therapy, and with Karnofsky performance status <90. Tumor volume was also larger in the TThP cohort, with 38% of patients having baseline volume >32 cc, compared to 15% and 19% for the SubT and LE-DE groups. MGMT status was unfortunately not known for 60% of patients.

Patients received up to 13 doses of VB-111. The median (mean) number of doses was 1 (2.2) and 4 (4.7) in the LE and TThP cohorts, respectively.

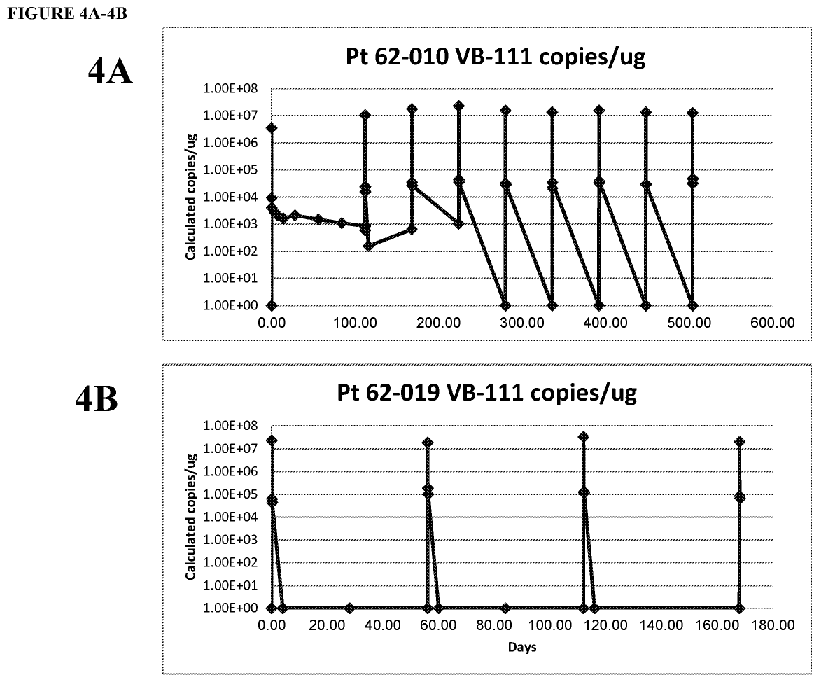

Biodistribution: Patients had a uniform peak of adenovirus DNA of approximately 10⁷ genome copies per microgram of genomic DNA in the blood immediately following VB-111 infusions, with no attenuation of peak levels with repeat dosing. All patients had some clearance of viral DNA levels within a few hours post-infusion, with a half-life time of 6 hours and a drop of at least 3 logs or more by Day 4 post-infusion with repeat dosing. This elimination of viral DNA in the whole blood indicated no accumulation of the virus in the blood and supports the safety of bi-monthly dosing. Some patients retained basal levels between doses only in the initial few doses, while in others, the levels between doses dropped to zero from the initial dosing. Examples of the two patterns of dose levels with repeat VB-111 dosing are shown in FIGS. 4A and 4B.

Safety and Tolerability: An MTD was not reached in the SubT cohort, as escalation proceeded to the maximum planned dose level (1×10¹³ VP) without observation of DLTs. The reported toxicities are detailed in Table 7.

TABLE 7
Adverse Events

| | LE Sub-Therapeutic | | LE Therapeutic | | TThp Therapeutic | |
|---|---|---|---|---|---|---|
| | Dose | | Dose | | Dose | |
| | (n = 16) | | (n = 22) | | (n = 24) | |
| Event | n (%) | | n (%) | | n (%) | |
| Any TEAE | 15 | (93.8) | 21 | (95.5) | 24 | (100) |
| Grade ≥3 TEAE | 2 | (12.5) | 9 | (40.9) | 4 | (16.7) |
| TEAE leading to discontinuation of | 0 | | 0 | | 2 | (8.3) |
| study drug | | | | | | |
| Infusion-related TEAEs^{a} | | | | | | |
| Fever | 3 | (19) | 11 | (50) | 14 | (58) |
| Flu like symptoms | 1 | (6) | 9 | (41) | 10 | (42) |
| Serious TEAE/Grade ≥3 TEAE | 2 | (12.5)/2 (12.5) | 9 | (40.9)/9 (40.9) | 10 | (41.7)/4 (16.7) |
| Blood and lymphatic system | 0 | | 0/1 | (4.5) | 0 | |
| (thrombocytopenia) | | | | | | |
| Eye disorder | 0 | | 1 | (4.5)/1 (4.5) | 0/1 | (4.2) |
| Gastrointestinal disorder | 0/1 | (6.3) | 0/1 | (4.5) | 0/1 | (4.2) |
| General disorders and administration | 0 | | 1 | (4.5)/2 (9.1) | 0 | |
| site conditions | | | | | | |
| Infections and infestations | 0 | | 1 | (4.5)/1 (4.5) | 1 | (4.2)/1 (4.2) |
| Musculoskeletal and connective tissue | 1 | (6.3)/0 | 2 | (9.1)/2 (9.1) | 0 | |
| disorders | | | | | | |
| Nervous system disorders | 1 | (6.3)/0 | 4 | (18.2)/5 (22.7) | 7 | (29.2)/6 (25.0) |

| | | | | | |
|---|---|---|---|---|---|
| Psychiatric disorders | 0 | 1 | (4.5)/1 (4.5) | 1 | (4.2)/1 (4.2) |

| | | | | | |
|---|---|---|---|---|---|
| Respiratory, thoracic and mediastinal | 2 | (12.5)/2 (12.5) | 0 | 1 | (4.2)/0 |
| disorders | | | | | |

| | | | | | |
|---|---|---|---|---|---|
| Vascular disorders | 0 | 1 | (4.5)/0 | 2 | (8.3)/3 (12.5) |

Approximately one-half of the patients developed fever and/or flu-like symptoms starting a few hours post-infusion; these events were generally self-limiting, grade 1-2 events and responded to antipyretic treatment. The rate of grade >3 treatment-emergent adverse events (AEs) ranged between 12% to 41% in the 3 cohorts, and, as expected, a large proportion were central nervous system related (i.e., neurological and psychiatric, up to 29%). Three vascular grade 3 events occurred, all in the TThP cohorts, including DVT (n=1) and hypertension (n=2). Two patients (8.3%) discontinued study treatment for toxicity.

Response and Tumor Growth Rate: In the SubT cohort, no significant antitumor activity was observed, with best response or stable disease (SD) by investigator and central review in 11 of 16 patients (69%) and 9 of 16 (56%) patients, respectively. In the LE cohort, confirmed partial response (PR) was noted in 1 of 19 patients (5%) by both investigator and central review, with stable disease in 9 patients (48%) and 12 patients (63%) by investigator and central review, respectively. No complete responses (CRs) were noted in either the SubT or LE cohorts. In the TThP cohort during monotherapy with VB-111, the following responses were noted by the investigator and central review, respectively: confirmed CR in 1 of 19 patients (5%) by both types of review; PR in 1 of 19 patients (5%) and 0 of 19 patients (0%), respectively; and SD in 10 of 19 patients (53%) and 11 of 19 patients (59%), respectively. In the TThP cohort during combined therapy, the following responses were noted by the investigator and central review, respectively: confirmed CR in 0 of 19 patients (0%) by both types of review, PR in 2 of 19 patients (10%) by both types of review; and SD in 10 of 19 patients (53%) and 9 of 19 patients (48%), respectively.

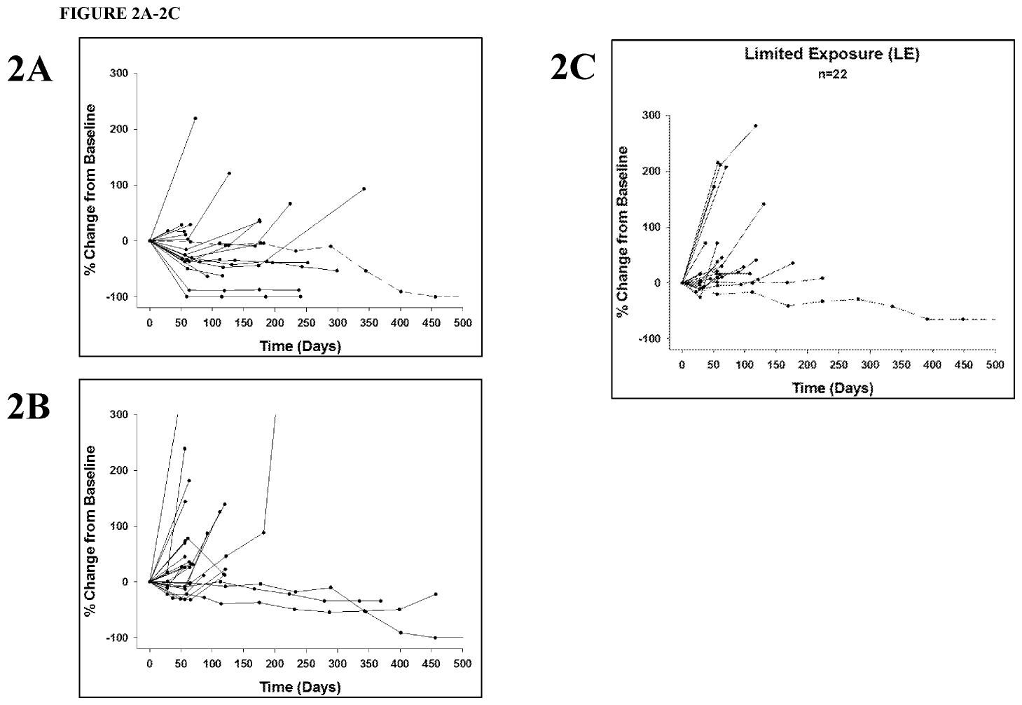

Spider diagrams demonstrated similarly rapid tumor growth in the LE and TThP cohorts during the VB-111 monotherapy period, with median percent increase (MPI) per 30 days by local site data of 14.8 vs. 14.1 (p=0.98) (FIGS. 2a and 2c). In the TThP cohort, growth was attenuated after the first progression compared to the preceding VB-111 monotherapy period (MPI: 0.6 vs. 14.1, p=0.0032) (FIGS. 2a and 2b). Responses seen in both the LE and TThP cohorts included 6 PRs, 2 of which achieved near-CR (98% reduction), and 1 CR maintained for over 3 years. A similar attenuation was seen in central laboratory tumor measurements. Tumor regression was more frequent in the TThP cohort: the proportion of patients with tumors decreased to below baseline dimensions during the first 100 days for 13% versus 61% of patients in the LE vs. TThP cohorts, respectively (p=0.002; McNemar's test).

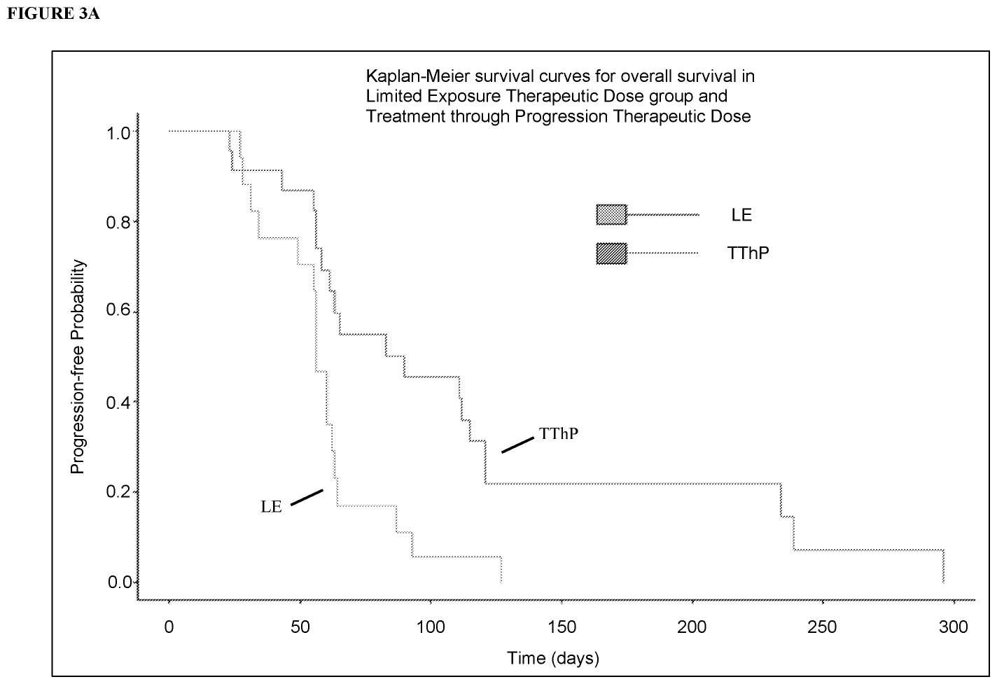

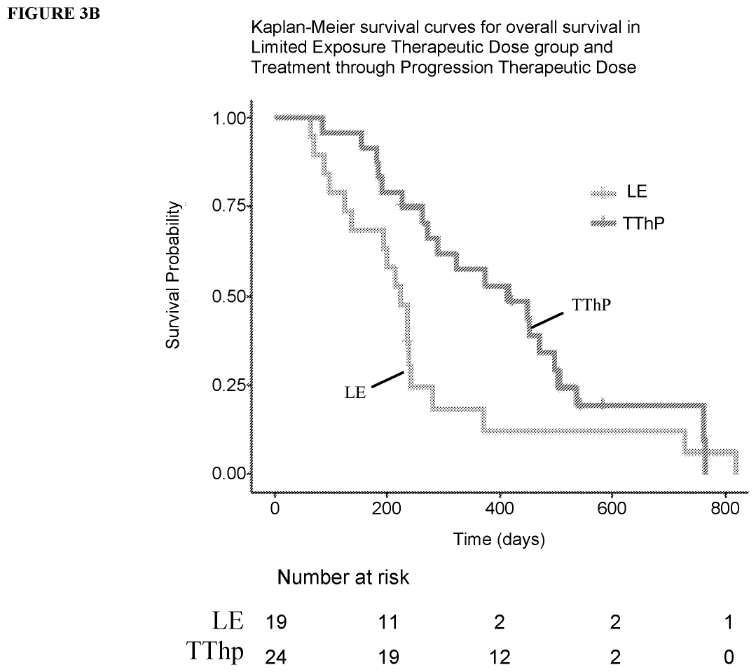

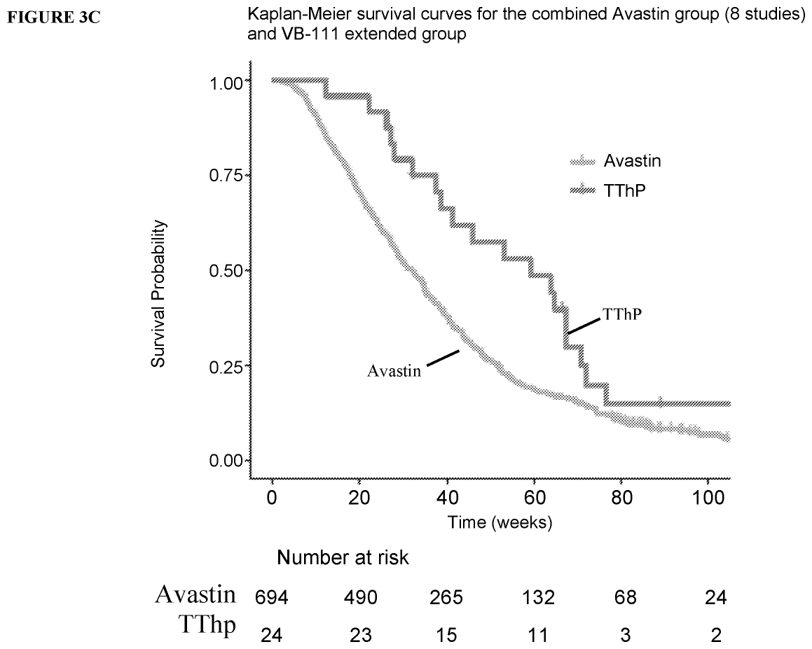

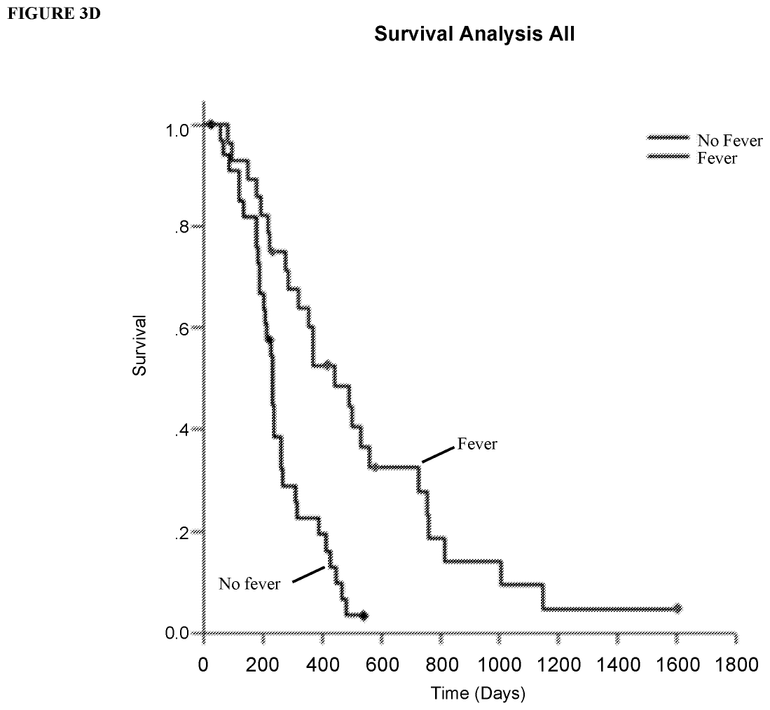

Survival: Median PFS by investigator assessment was 55, 56, and 61 days for the SubT, LE, and TThP monotherapy cohorts, respectively (p=not significant [NS]). The median PFS in the TThP combination cohort (from start of combination therapy until disease progression) was 83 days (vs. 56 days for the LE cohort, hazard ratio [HR] 0.46 [95% confidence interval (CI) 0.23-0.91]; p=0.01 [log-rank]; FIG. 3a). Central review showed similar median PFS of 55, 60, and 61 days for the SubT, LE, and TThP monotherapy cohorts, respectively (p=NS).

Overall survival was 38 weeks (266 days), 32 weeks (223 days), and 59 weeks (414 days) in the SubT (n=16), LE (n=19), and TThP (n=24) cohorts, respectively; OS in the TThP cohort was significantly greater than the LE cohort (HR 0.51 [95% CI 0.26-0.99]; p=0.043 [log-rank]; FIG. 3b).

A historical control group was constructed based on a meta-analysis of 8 publications including 694 rGBM patients treated with bevacizumab monotherapy. Median OS for the historical group was 32 weeks, which is significantly lower than the OS in the TThP cohort (HR 0.62 [95% CI 0.40-0.96]; p=0.029 [log-rank]; FIG. 3c). The 12-month OS rate was 57% vs. 24% in the VB-111 TThP cohort vs. the pooled meta-analysis, respectively (p=0.002). After omitting the Duerinck et al 2015 study from the historical cohort, the median OS was 37 weeks, still significantly lower than the OS in the TThp cohort (p=0.038 [log-rank]), and the 12-month OS rate was 30%, still significantly lower than that of the TThp cohort (p=0.005).

Development of a febrile reaction post-infusion occurred in 45% of patients and was associated with improved survival (all cohorts; FIG. 3d), with an OS of 235 vs. 448 days (p<0.001). This was true for the SubT (OS 229 vs. 561, p=0.027) and LE-DE (OS 194 vs. 371, p<0.001) cohorts, but not for the TThP cohort (OS 321 vs. 448, p=NS)

The median OS in the TThP group of 59 weeks was statistically significantly better than the LE cohort survival (32 weeks), all historical controls available, and the composite median OS from 8 studies in the pooled meta-analysis (32 weeks). Importantly, the percentage of patients living beyond 1 year in the TThP cohort (57%) was more than double that in the pooled meta-analysis (24%), with all but 1 patient followed to death or a minimum of 15 months. The OS benefit was greater than the PFS signal, which was modest but reached statistical significance, and individual patient tumor growth data suggests significant attenuation in tumor growth.

VB-111 was very well tolerated both as a single agent and in combination with bevacizumab. The grade 3 or higher treatment-related AE rate of 17% in the TThP cohort compares favorably with single-arm studies, and is significantly lower than that reported for bevacizumab in combination with either lomustine or irinotecan. One of the most common AEs of any grade associated with VB-111 was a febrile response typically occurring several hours after the infusion and resolving by the following day, which occurred in 50% of patients in the LE cohort and 58% in the TThP cohort. The development of a febrile response was highly correlated with improved survival.

The results of this study show that VB-111 given every 56 days improves survival in rGBM when administered with bevacizumab and is associated with a favorable safety profile.

### Example 3

### Ad5-PPE-1-3X-Fas-c Therapy Induces Anti-Tumoral Immunotherapeutic Responses

Background: Ad5-PPE-1-3X-Fas-c has shown a favorable toxicity profile and efficacy in phase 2 clinical studies for several cancer indications, including nearly doubling of overall survival (OS) in rGBM when added to bevacizumab. As shown in Example 2, an increased OS is more prominent in subjects experiencing febrile reaction to Ad5-PPE-1-3X-Fas-c. The present example discloses methods of treating a tumor in a subject who is capable of exhibiting a change in at least one plasma biomarker or cell surface biomarker after administration of at least one priming dose of Ad5-PPE-1-3X-Fas-c, as well as methods of identifying a responder to Fas-chimera gene therapy.

Methods: (i) Serum from the rGBM patients at baseline and 6 hours post Ad5-PPE-1-3X-Fas-c infusion was subjected to cytokine profiling using a luminex based array. (ii) Pathological specimens from Ad5-PPE-1-3X-Fas-c treated and control platinum-resistant ovarian cancer patients were stained for CD8 T-cells. (iii) Mice with orthotopic U251 tumors were treated with Ad5-PPE-1-3X-Fas-c or control. Sorted tumor microglia and T-cells were co-cultured with splenocytes and activated with LPS or anti CD3/CD28. Supernatants were subjected to cytokine profiling. (iv) Combination treatment of anti-PD-L 1 and Ad5-PPE-1-3X-Fas-c in animal models was performed.

Results: (i) In the rGBM patients, levels of several cytokines 6 hours post Ad5-PPE-1-3X-Fas-c infusion correlated with OS. (ii) Immunohistochemistry showed profound increase in tumor-infiltrating CD8 T-cells in ovarian cancer patients treated with Ad5-PPE-1-3X-Fas-c compared with control specimens. (iii) When tumor microglia and T-cells from the Ad5-PPE-1-3X-Fas-c-treated U251 model were co-cultured with splenocytes and activated with LPS or anti CD3/CD28, a significant increase in different cytokines and chemokines was observed only in co-cultures with microglia. Conversely, in anti-CD3/CD28 activated splenocytes and tumor T-cell co-culture, a decrease in anti-inflammatory cytokines was observed. These results suggest that tumoral microglia are responsible for cytokine release with a profile that promotes a cytotoxic T-cell response. (iv) In animal studies performed with Lewis lung carcinoma and melanoma, a reduction of tumor volume and increase in CD8 cells was seen with the combination of a single dose of Ad5-PPE-1-3X-Fas-c and anti PD-L1.

Conclusions: In addition to the previously described antiangiogenic mechanism of Ad5-PPE-1-3X-Fas-c, these data show an immune-activating role of Ad5-PPE-1-3X-Fas-c acting as a viral immune oncology agent.

### Example 4

### Data Set Analysis for Ad5-PPE-1-3X-Fas-c Therapy, Cytokine Measurement, and Overall Survival

Analysis was performed using a Luminex assay of serum samples from the Ad5-PPE-1-3X-Fas-c clinical data set described above. The relationship of 27 cytokines and overall survival (OS) was assessed. The treatment groups were categorized as “Continuous” or “Limited.” Subjects in the Continuous treatment group received treatment with VB-111, and upon progression, continued treatment with VB-111 in combination with bevacizumab, regardless of disease status. Subjects in the Limited treatment group received VB-111 as monotherapy, and upon disease progression, switched to bevacizumab as the standard of care.

Data processing and transformation. To create the complete data set, the results from two plate's runs by Luminex cytokine assay (Millipore) were combined. Additional to cytokine levels data were added variables such as “OS,” “group,” “fever at 1st dose,” fever at any dose.”

After verifying the quality of the data, the values, which were below detectable level, were replaced to median values from 0 to detectable level.

Because each cytokine was analyzed at two time points (0 and 6 hours after Ad5-PPE-1-3X-Fas-c infusion) we used delta for cytokine levels which represent the dynamical changes in cytokine levels 6 hours after Ad5-PPE-1-3X-Fas-c treatment.

The statistical analyses were performed by Matlab and R programming language.

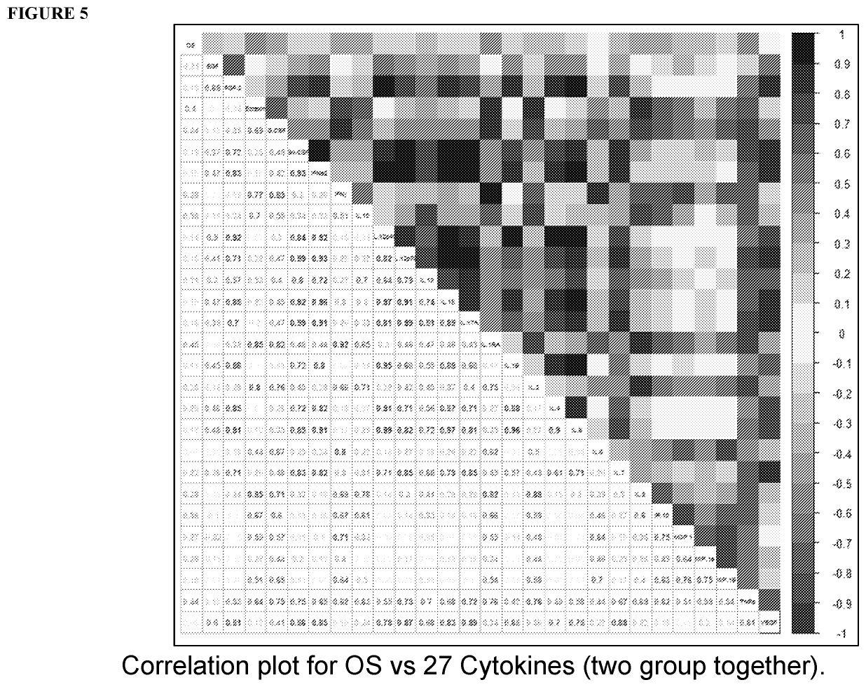

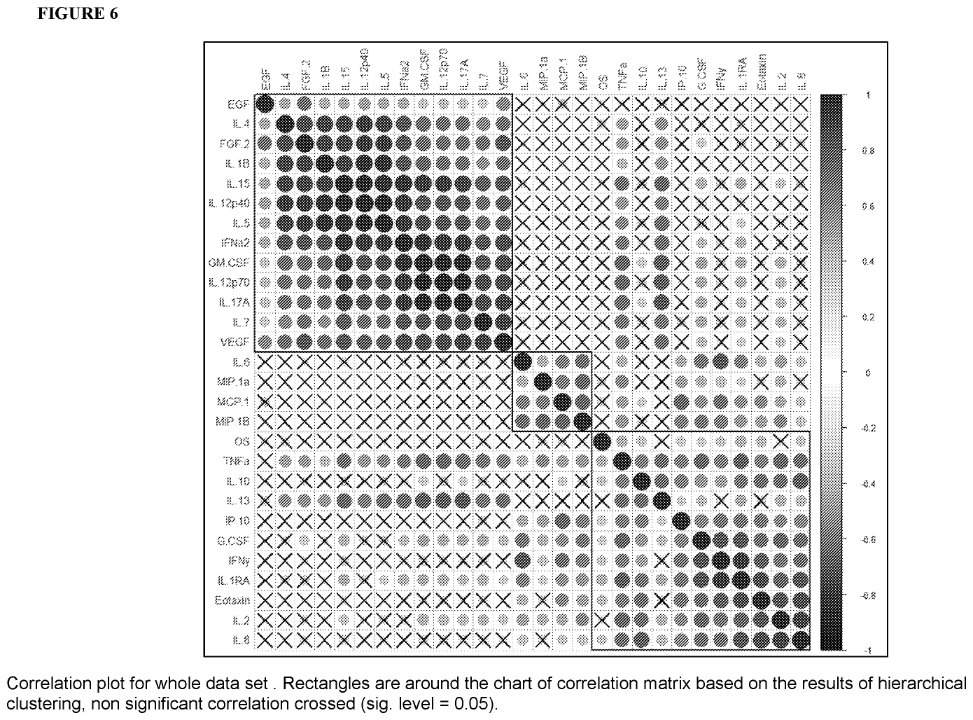

Exploratory data analysis. Initially, to examine the relationship between OS and changes in 27 cytokines levels 6 hours after drug administration, a correlation plot was made (FIG. 5). Based on Pearson correlation coefficient, the strongest correlation was identified between OS and such cytokines as IL-1Ra (+0.46), TNFα (+0.44) and Eotaxin (+0.4). The relationship between OS and additional cytokines IFNγ, IL-10, IL-8, IP-10 and G-CSF was also assessed. By significance test (sig. level >0.05), it was determined that all 8 cytokines (IL-1Ra, TNFα, Eotaxin, IFNγ, IL-10, IL-8, IP-10 and G-CSF) have a statistically significant correlation with OS (FIG. 6).

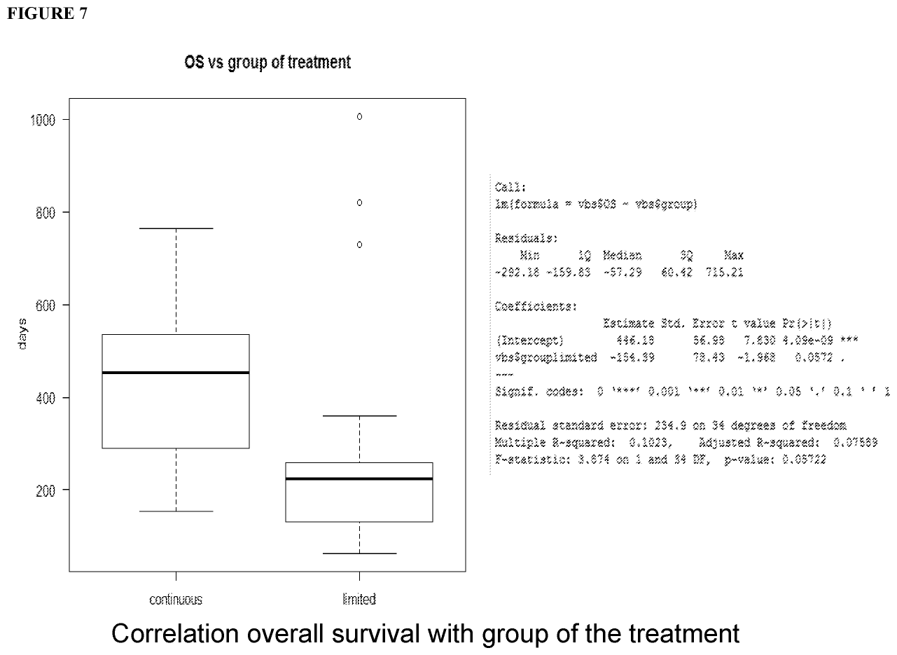

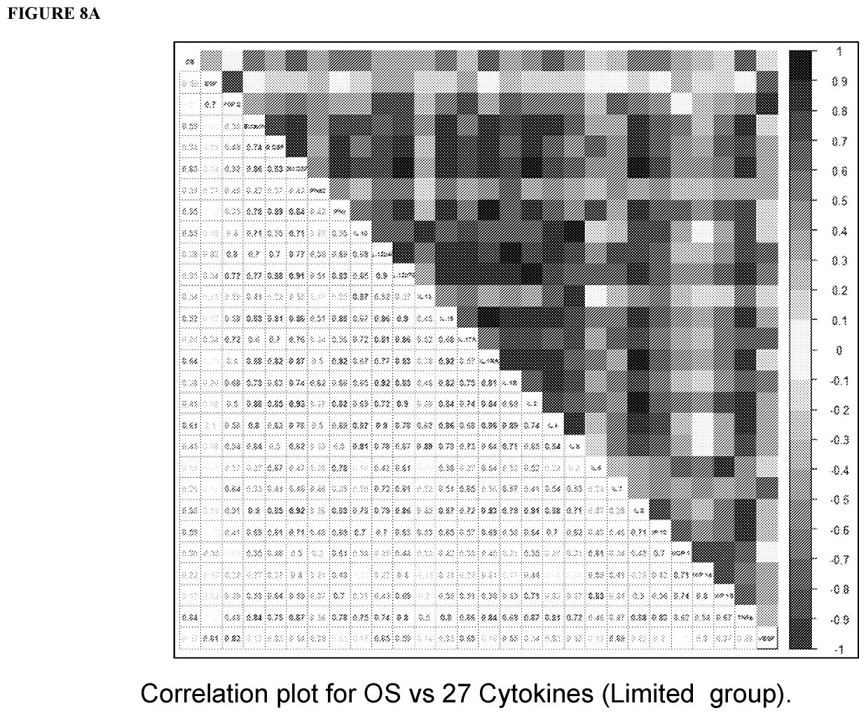

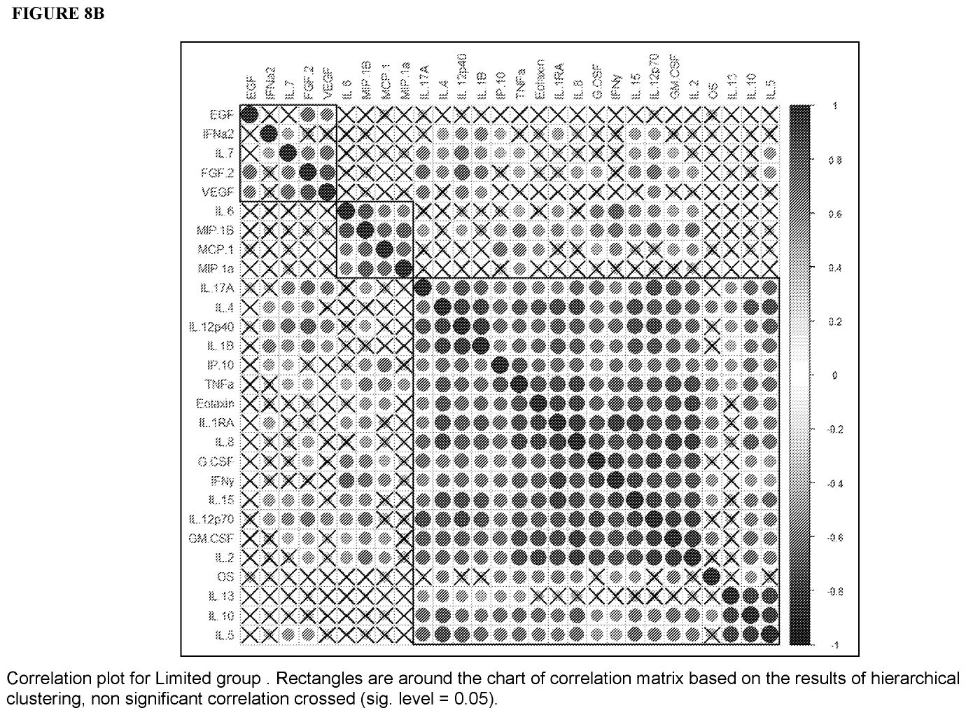

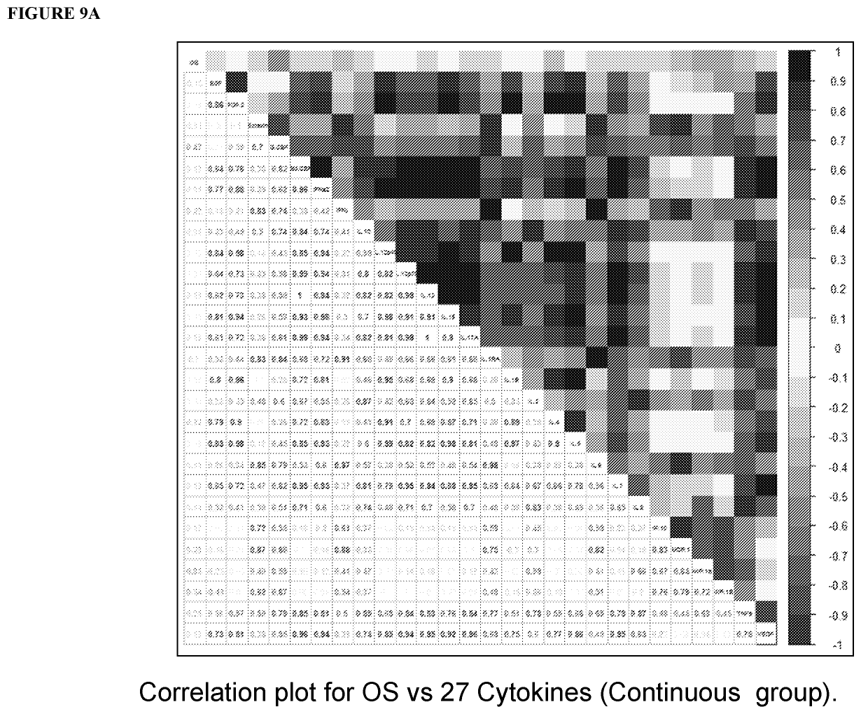

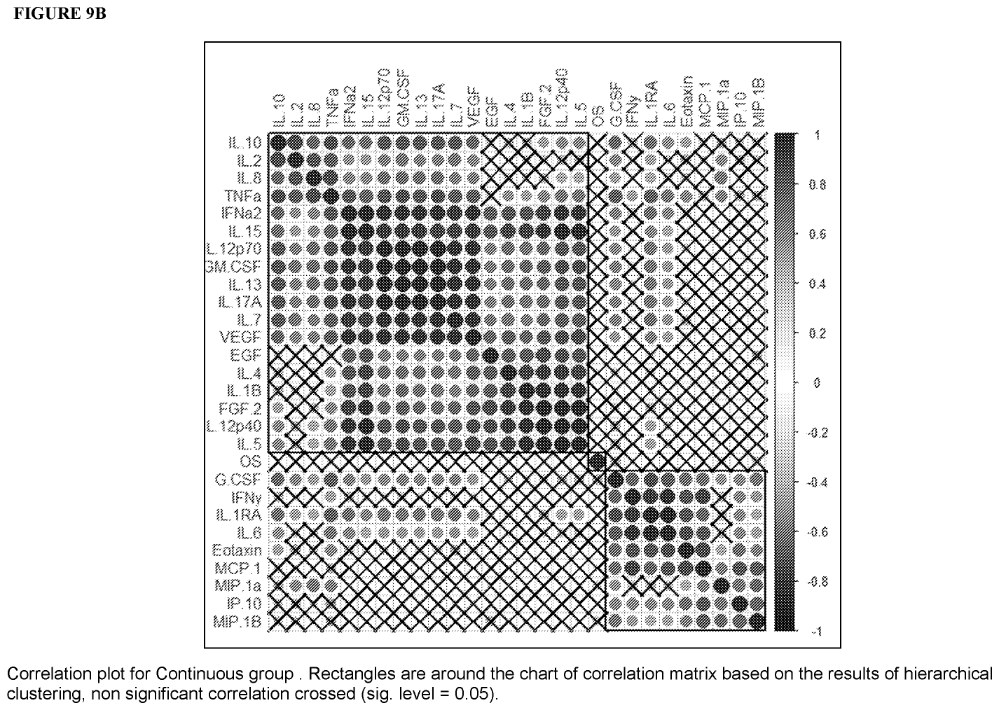

Given the fact that the survival rate was significantly different between two treatment groups (FIG. 7), the correlation between OS and cytokines depending on the treatment group was assessed (FIG. 8-9). It was determined that OS for the Limited group has the strongest correlation with the 8 cytokines compared to the two groups combined together. It was also determined that OS for the Limited group correlates with increases in cytokines IL-15 and IL-4 (FIG. 8A), and that the correlation is significant (FIG. 8B). The OS for the Continuous group did not have a significant correlation with changes of any of the 27 examined cytokines (FIG. 9A, 9B).

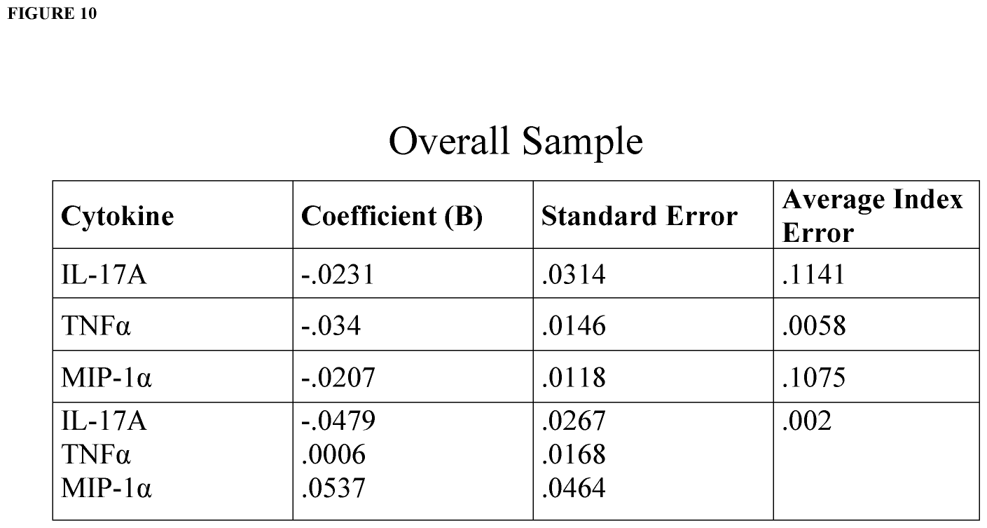

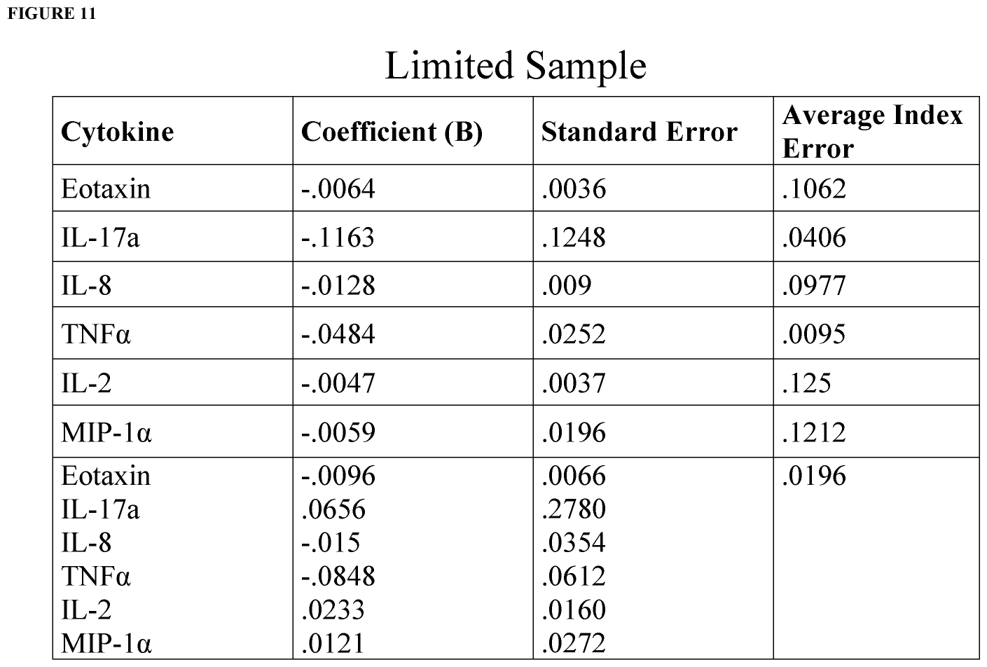

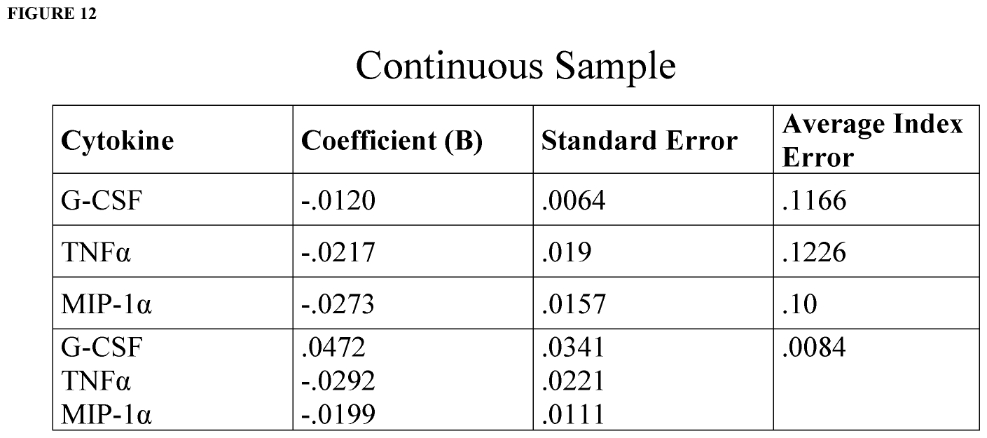

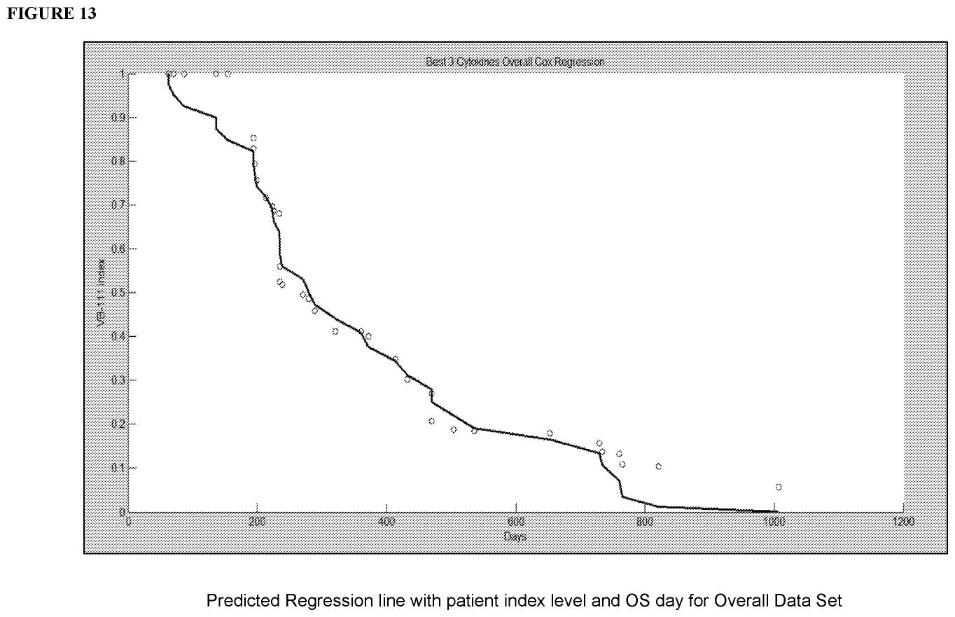

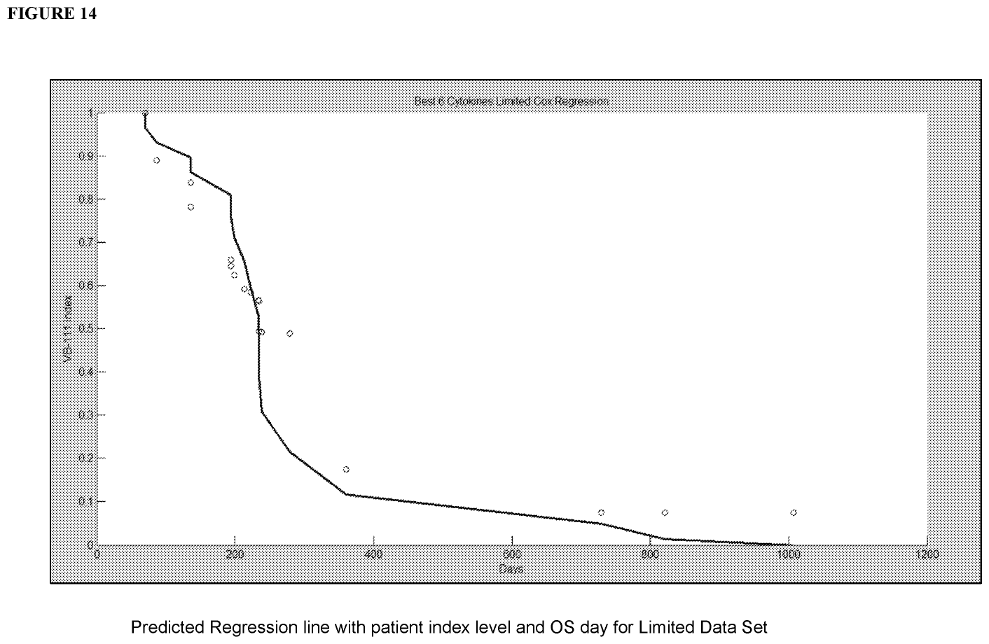

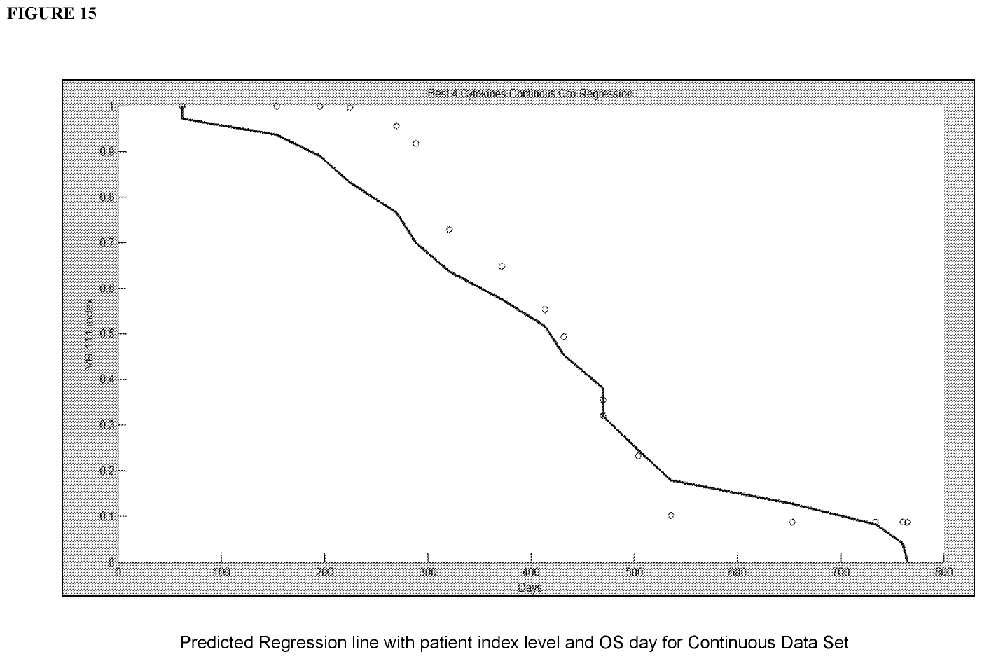

Cox proportional hazard analysis. A Cox regression analysis was used. A Cox Regression is a type of logistic regression in which it looks for a time of event, in this case death. The Cox Regression was performed on the cytokines mention above in three separate sets, overall, continuous and limited, to see if there was any variation between them. The Regression provides an estimate line to follow to determine the OS from the index of the patient. Along with performing the regression on all the cytokines, a multivariate Regression was performed with the best cytokines to get a better prediction assigning different coefficients for each cytokine than they will have on their own. A baseline Equation is needed when using Cox Regression and for simplicity the mean was used. FIG. 10 (Overall Sample), FIG. 11 (Limited treatment group samples), and FIG. 12 (Continuous treatment group samples) each shows three values: the coefficient that the Cox Regression assigned to the cytokine, its standard Error for the coefficient, and an average index error. FIG. 13, FIG. 14, and FIG. 15 each shows the estimated line plotted against the actual values to get an idea of the error.

Conclusion. Based on the observation from biostatistical analysis, changes in the levels of 4 cytokines showed the greatest correlation with the OS of a patient. IL-1Ra and TNFα correlated with OS in T testing, TNFα, IL-17a, and MIP-1α correlated in all data sets in Cox Regression. Other Cytokine levels that do not change much after Ad5-PPE-1-3X-Fas-c but maintain a correlation to OS can also be informative.

### Example 5

In this example, Mice with orthotopic U251 tumors were treated with Ad5-PPE-1-3X-Fas-c or control. Sorted tumor microglia and T-cells were co-cultured with splenocytes and activated with LPS or anti CD3/CD28. Culture supernatants were subjected to cytokine profiling.

Methods: Mice harboring orthotopic U251 tumors were treated with injected intravenously with 1×10¹¹ virus particles of Ad5-PPE-1-3X-Fas-c. Six hours after treatment, animals were sacrificed and tumors and spleens were removed. Cells from the tumor and splenocytes were harvested from respective tissues using Accumax cell dissociation solution.

Cells extracted from the tumor were incubated with FcR blocking buffer for 15 minutes. Following a wash with PBS, samples were incubated for 20 minutes with the following antibodies: Zombie-NIR for live cells; CD45-labeled with APC (Allophycocyanin) for non-tumor cells; CD11b-labeled with PerCP (Peridinin-chlorophyll Protein) for microglia; CD3-labeled with BV421(Brilliant Violet 421) for total T cells.

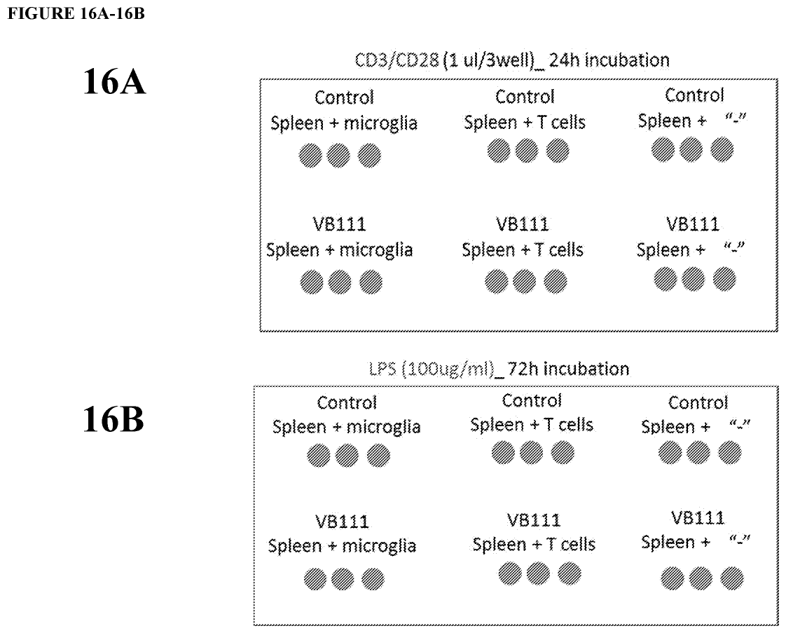

Labeled cells were counted and seeded in triplicate wells in 96-well plates (200 microliters of medium per well). Spleen cells were seeded at a density of 2.8×10⁴ cells per well. Microglial cells were seeded at a density of 1×10⁵ cells per well. T-cells were seeded at a density of 2.4×10⁴ cells per well. Cells were co-incubated with either anti-CD3/anti-CD28 for 24 hours (FIG. 16A) or LPS at 100 μg/ml for 72 hours (FIG. 16B). After incubation, culture supernatants were collected and stored at −20° C. until analysis.

Cytokines were profiled using Luminex cytokine assay (Millipore; 32 cytokines, mouse) in triplicate wells.

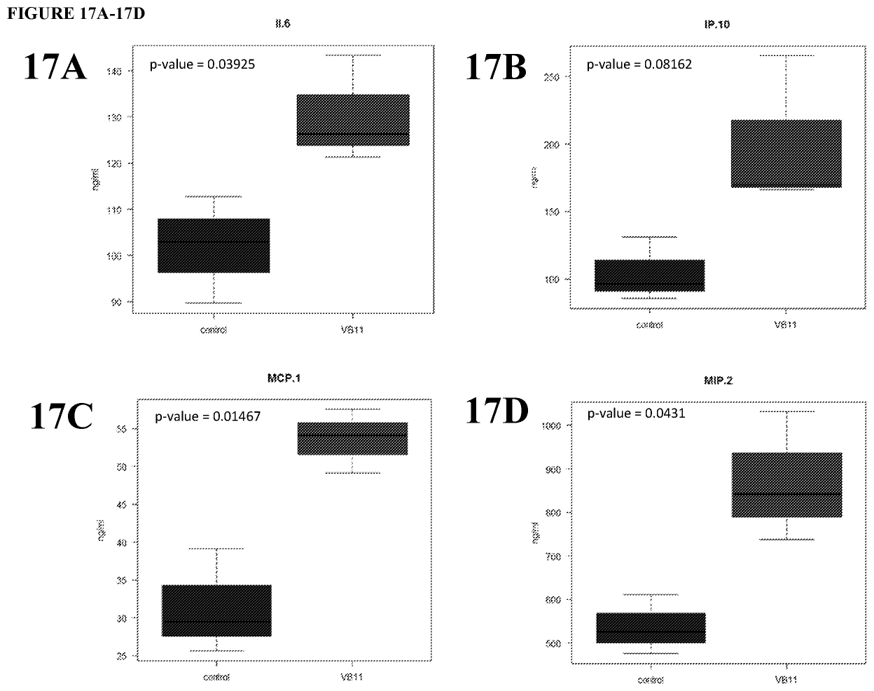

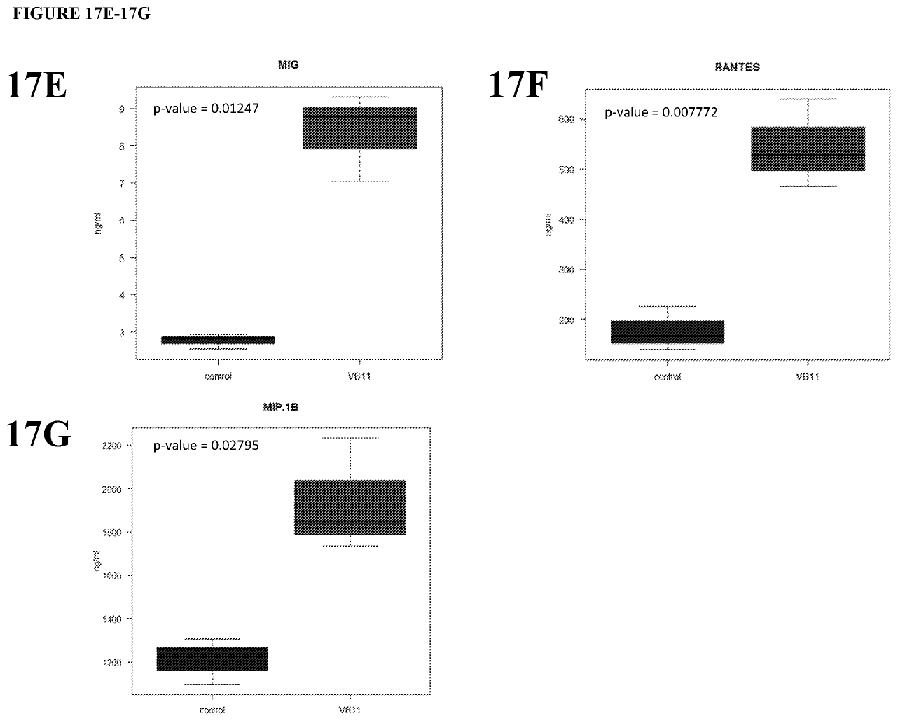

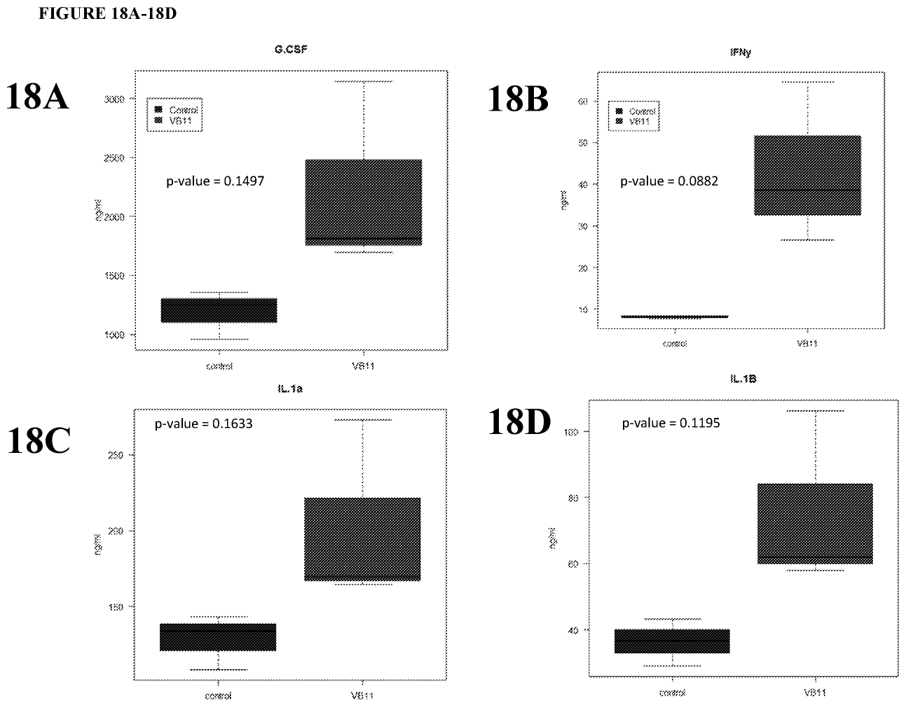

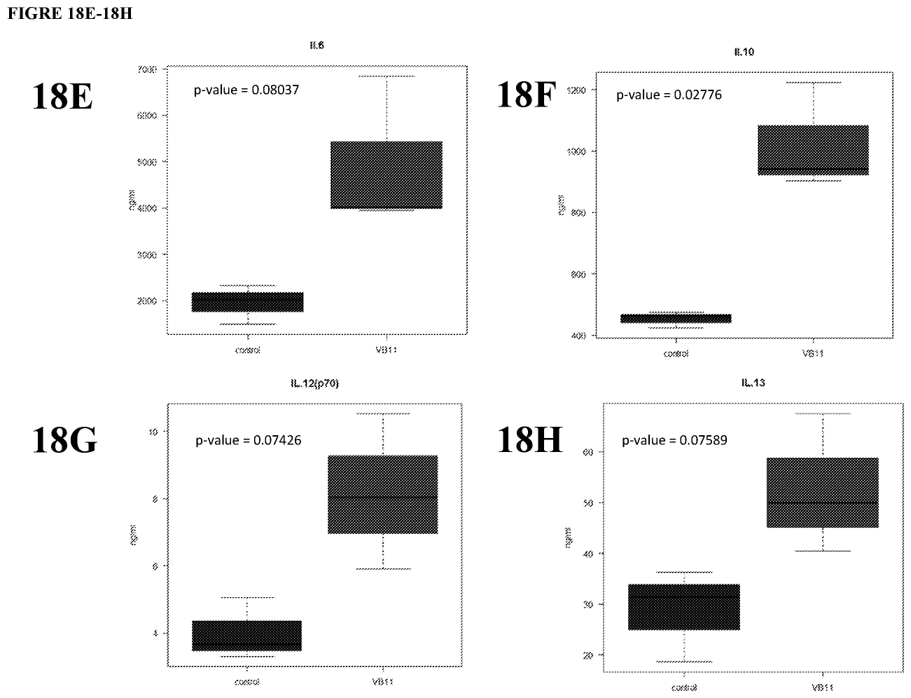

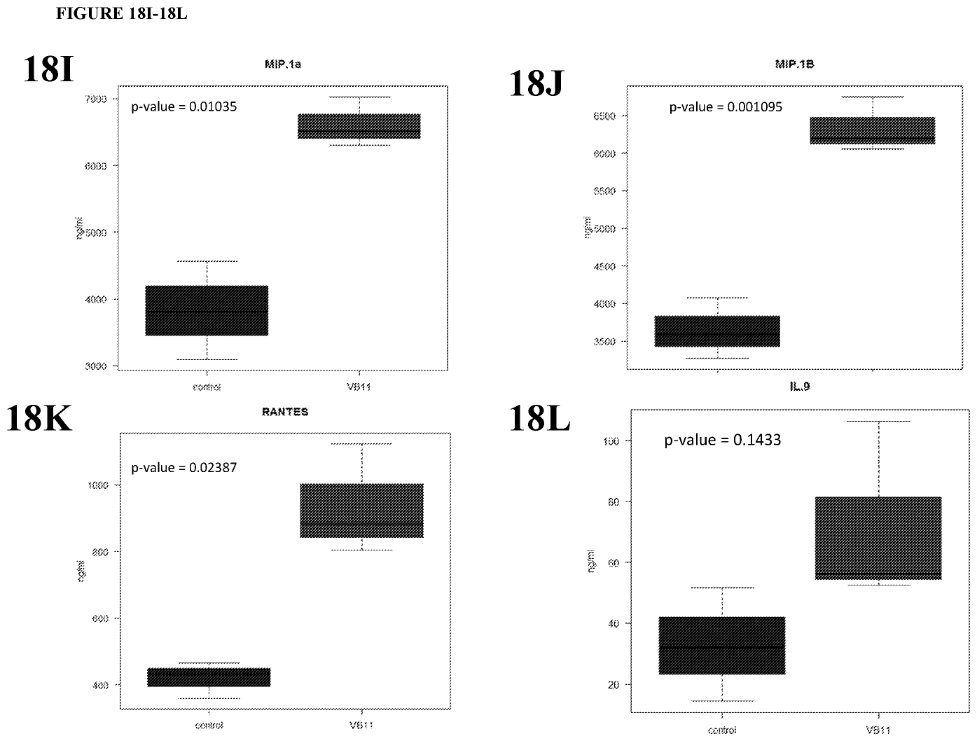

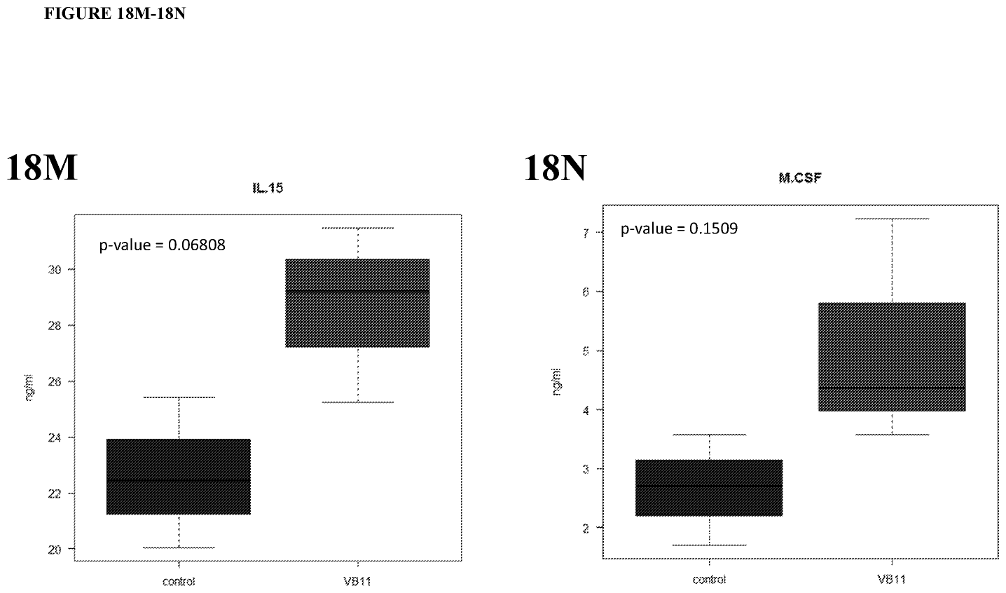

Results: When tumor microglia and T-cells from the Ad5-PPE-1-3X-Fas-c-treated U251 model were co-cultured with splenocytes and activated with LPS or anti CD3/CD28, a significant increase in different cytokines and chemokines was observed only in co-cultures with microglia. For example, FIG. 17A-17G show increased levels of IL-6, IP-10, MCP-1, MIP-2, MIG, RANTES, and MIP-1β, respectively, present in the supernatant from spleen and microglial cells co-cultured with anti-CD3/anti-CD28 in samples from animals treated with Ad5-PPE-1-3X-Fas-c compared with controls. FIG. 18A-18N show increased levels of G-CSF, IFN-γ, IL-1α, IL-1β, IL-6, IL-10, IL-12 (p70), IL-13, MIP-1α, MIP-1β, RANTES, IL-9, IL-15, and M-CSF, respectively, present in the supernatant from spleen and microglia cells co-cultured with LPS in samples from animals treated with Ad5-PPE-1-3X-Fas-c compared with controls.

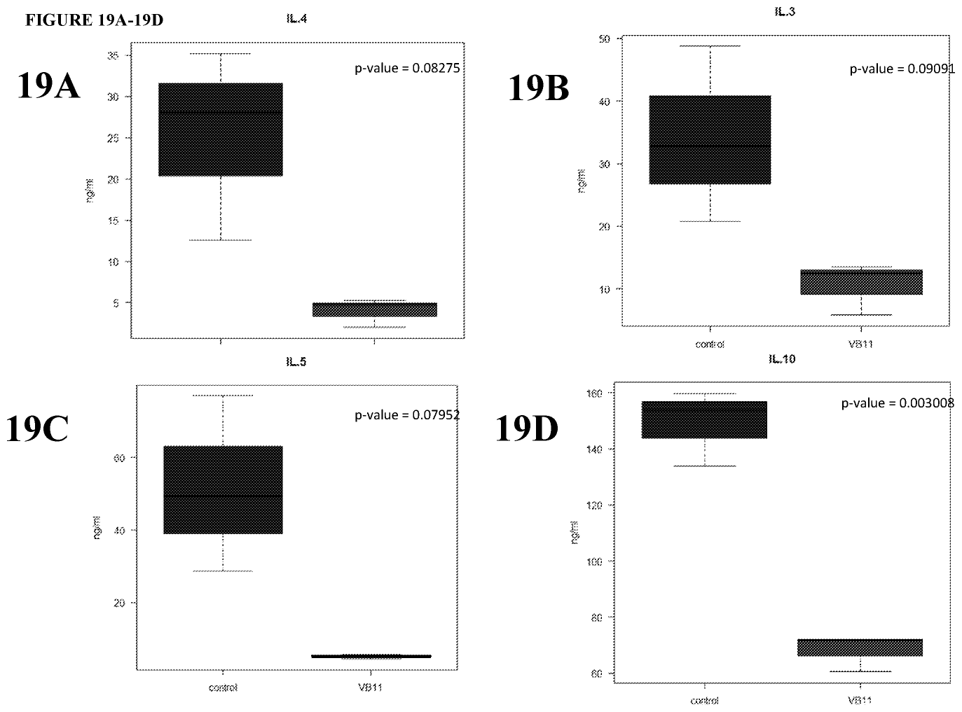

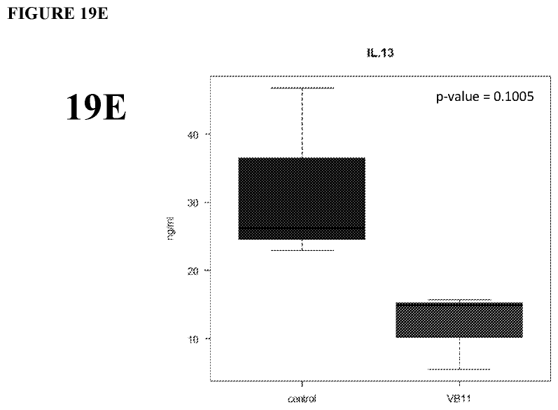

Conversely, in anti-CD3/anti-CD28 activated splenocytes and tumor T-cell co-culture, a decrease in anti-inflammatory cytokines was observed. For example, FIG. 19A-19E show decreased levels of IL-4, IL-3, IL-5, IL-10, and IL-13, respectively, in the supernatant from spleen and T-cells co-cultured with anti-CD3/anti-CD28 in samples from animals treated with Ad5-PPE-1-3X-Fas-c compared with controls.

These results show that tumoral microglia are major factors in cytokine release with a cytokine profile that promotes a cytotoxic T-cell response.

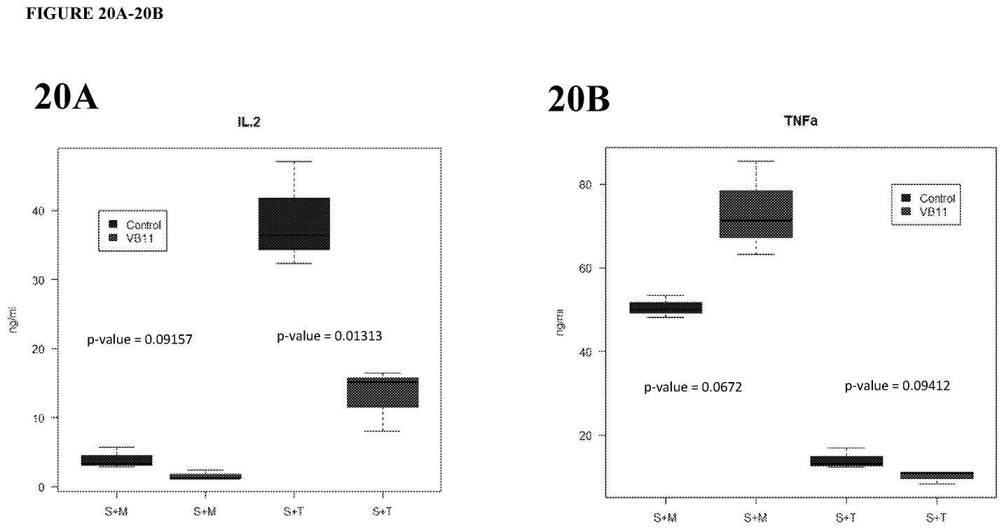

Some cytokines were detected when splenocytes were incubated with microglia, but not when splenoctyes were incubated with T-cells, and vice versa. For example, FIG. 20A shows low levels of IL-2 were detected in both treated and control samples when splenocytes were incubated with microglia (S+M). However, when splenocytes were incubated with T-cells (S+T), increased levels of IL-2 were detected in control samples and reduced levels of IL-2 were detected in samples from Ad5-PPE-1-3X-Fas-c-treated animals. See FIG. 20A. FIG. 20B shows increased levels of TNFα were detected in samples from Ad5-PPE-1-3X-Fas-c-treated animals compared to controls when splenocytes were incubated with microglia (S+M). However, when splenocytes were incubated with tumor-infiltrating T-cells (S+T), decreased levels of TNFα were detected in both treated and control samples. See FIG. 20B.

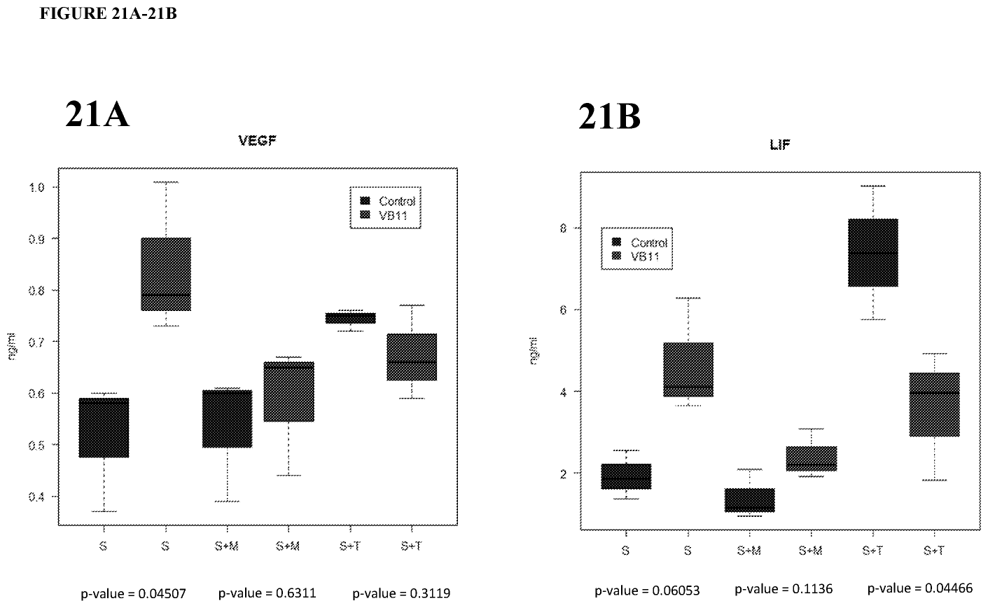

The levels of VEGF and Leukemia Inhibitory Factor (LIF) were also assessed. For example, the levels of VEGF and LIF were measured from the supernatants of cultured splenocytes only (S), splenocytes co-cultured with microglia (S+M), and splenocytes co-cultured with tumor infiltrating T-cells (S+T). Results are shown in FIG. 21A and FIG. 21B. FIG. 21A shows that splenocytes from Ad5-PPE-1-3X-Fas-c-treated animals produced increased levels of VEGF upon stimulation with anti-CD3/anti-CD28 compared with control samples. VEGF production in splenocytes co-cultured with microglia or tumor infiltrating T-cells was similar in treated and control samples. See FIG. 21A. FIG. 21B shows that splenocytes from Ad5-PPE-1-3X-Fas-c-treated animals produced increased levels of LW upon incubation with anti-CD3/anti-CD28 compared with control samples. LIF levels were decreased in samples from Ad5-PPE-1-3X-Fas-c-treated animals when splenocytes were incubated with tumor-infiltrating T cells. See FIG. 21B.

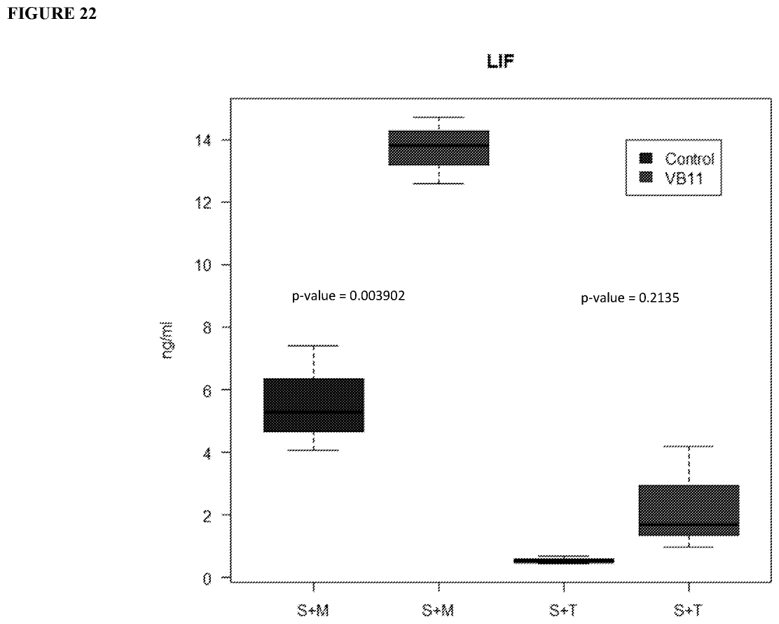

A similar experiment was performed using LPS for stimulation. FIG. 22 shows that LIF levels were increased in samples from Ad5-PPE-1-3X-Fas-c-treated animals when splenocytes were co-cultured with microglia (S+M), but not significantly when splenocytes were co-cultured with tumor T-cells (S+T).

### Example 6

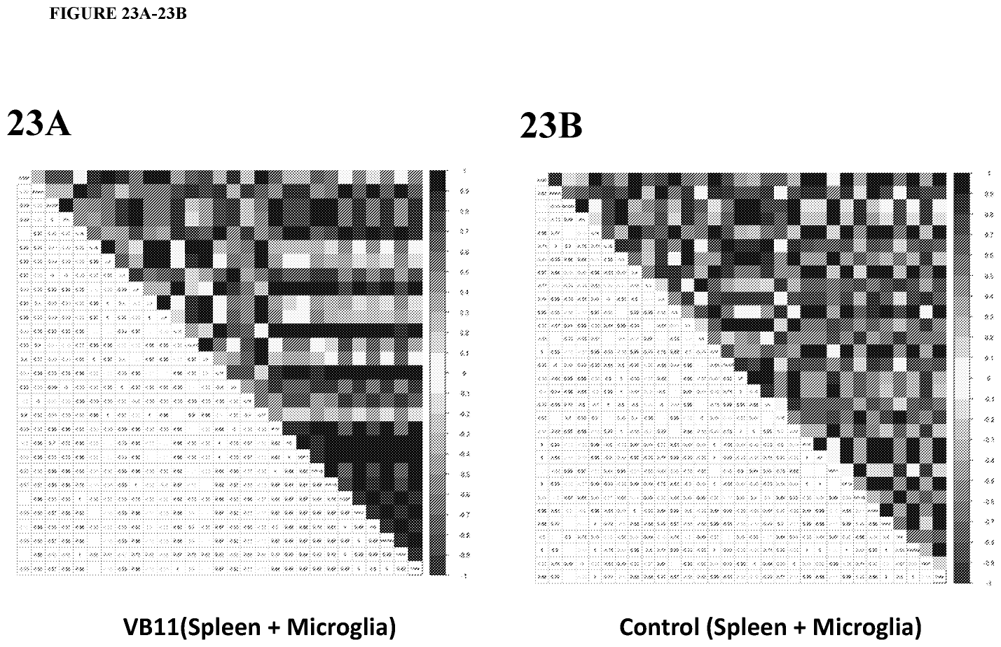

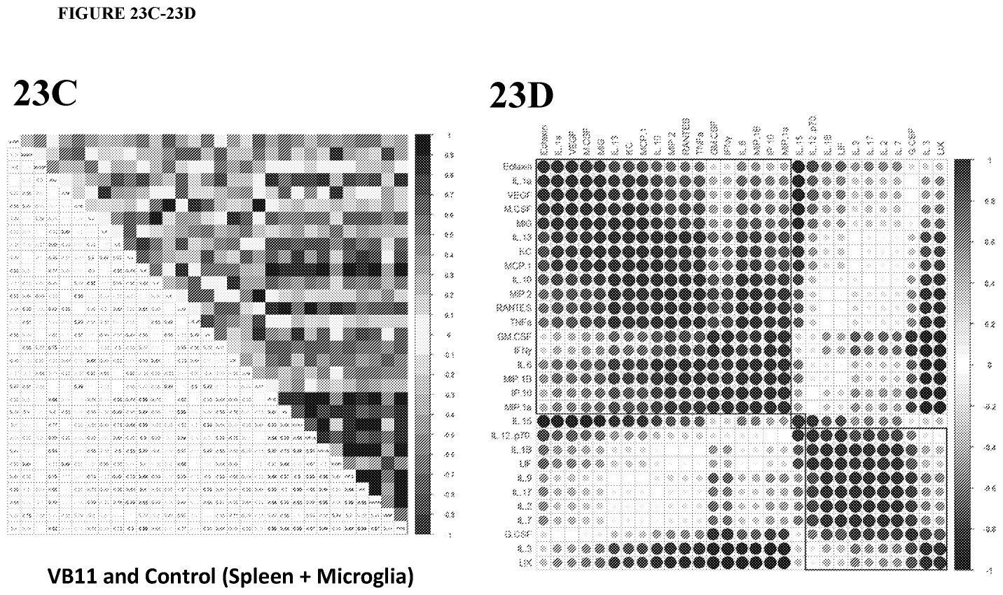

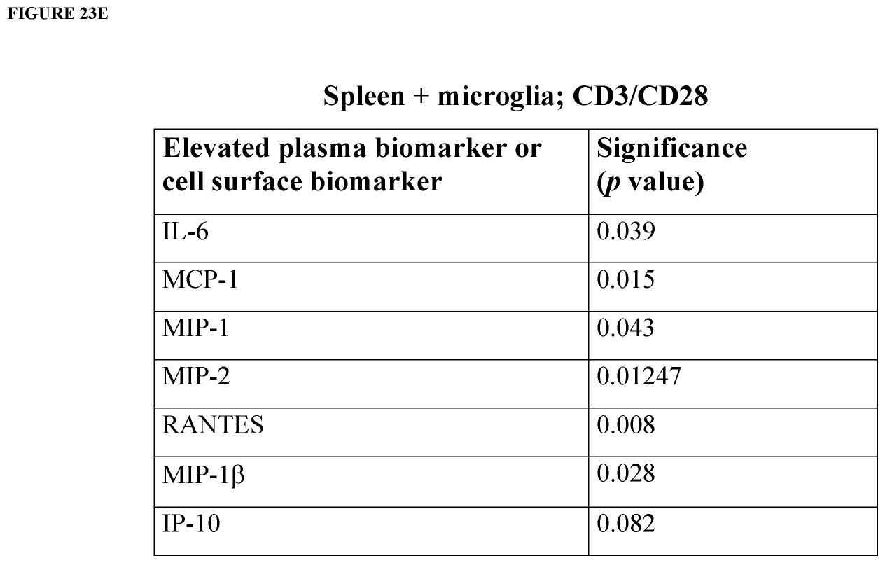

After performing the cytokine profile analyses in Example 5, a correlation was identified between significant cytokine changes in splenocytes co-cultured with microglia and stimulated with anti-CD3/anti-CD28. The results are shown in FIG. 23A-23E. FIG. 23A-23C show the levels of 27 cytokines/chemokines that were measured in the supernatant of each assay for Ad5-PPE-1-3X-Fas-c-treated animals (FIG. 23A), control animals (FIG. 23B), and both Ad5-PPE-1-3X-Fas-c-treated animals and control animals (FIG. 23C). The intensity in RED or BLUE indicates the level of correlation—a darker color indicates a stronger correlation. FIG. 23D shows another way to plot the merger data as in FIG. 23C. The cytokines were clustered into 3 groups. Cytokines that have shown significance change compared to the control are combined into one cluster. The most significant cytokines from this experiment were determined to be IL-6, MCP-1, MIP-1α, MIP-2, RANTES, MIP-1β, and IP-10 (see FIG. 23E).

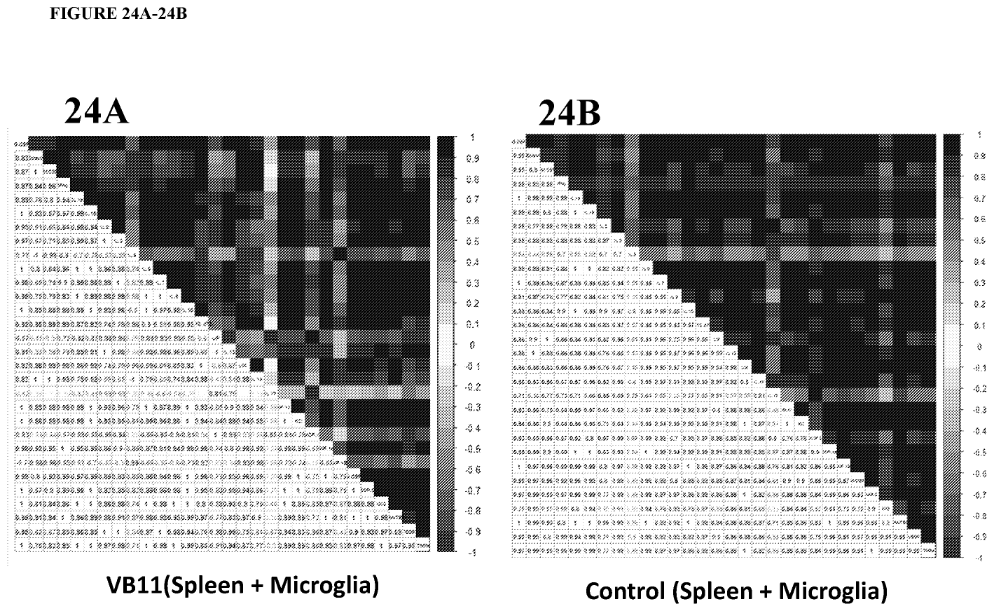

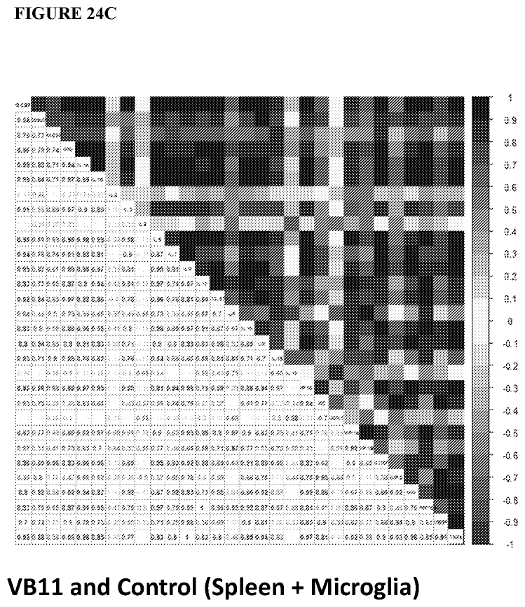

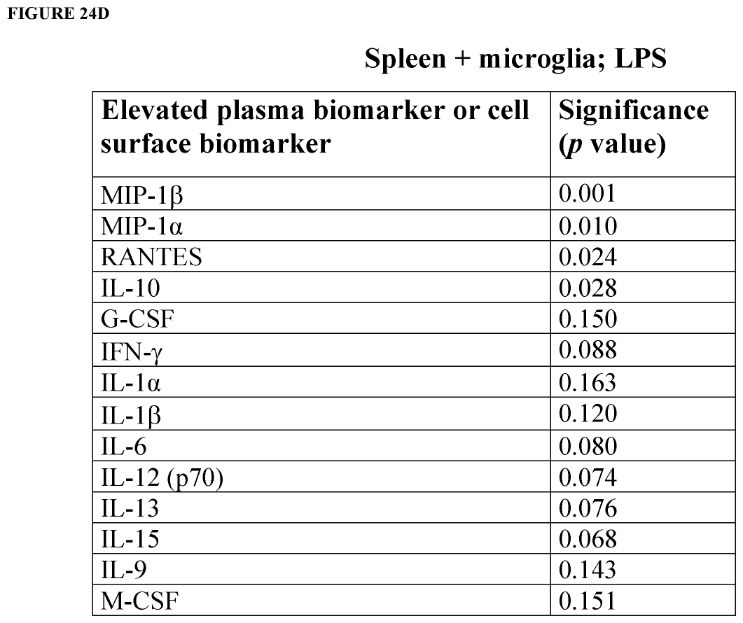

A similar correlation study was performed to identify significant cytokine changes in splenocytes co-cultured with microglia and stimulated with LPS. The results are shown in FIG. 24A-24D. FIG. 24A-24C show the levels of 27 cytokines/chemokines that were measured in the supernatant of each assay for Ad5-PPE-1-3X-Fas-c-treated animals (FIG. 24A), control animals (FIG. 24B), and both Ad5-PPE-1-3X-Fas-c-treated animals and control animals (FIG. 24C). The most profoundly changed cytokines from this experiment were determined to be MIP-1β, MIP-1α, RANTES, IL-10, G-CSF, IFN-γ, IL-1α, IL-1β, IL-6, IL-12 (p70), IL-13, IL-15, IL-9, and M-CSF (see FIG. 24D).

### Example 7

The correlations between overall survival (OS), changes in at least one plasma biomarker or cell surface biomarker, and development of fever were assessed. Analysis was performed using samples from the Ad5-PPE-1-3X-Fas-c clinical data set described above in Example 4.

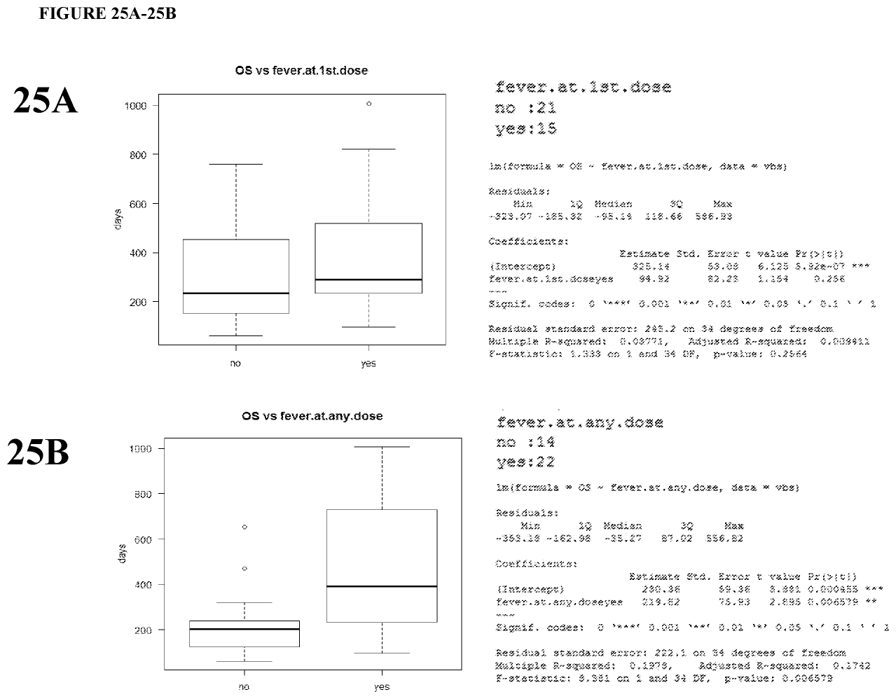

First, the correlation between OS and subjects exhibiting a fever (either after the priming dose of vector or after any dose of vector) was assessed. FIG. 25A shows that subjects exhibiting a fever after the priming dose of vector had an increased OS compared to subjects who did not exhibit a fever after the priming dose. FIG. 25B shows that subjects exhibiting a fever after any dose of vector had an increased OS compared to subjects who did not exhibit a fever after any dose of the vector.

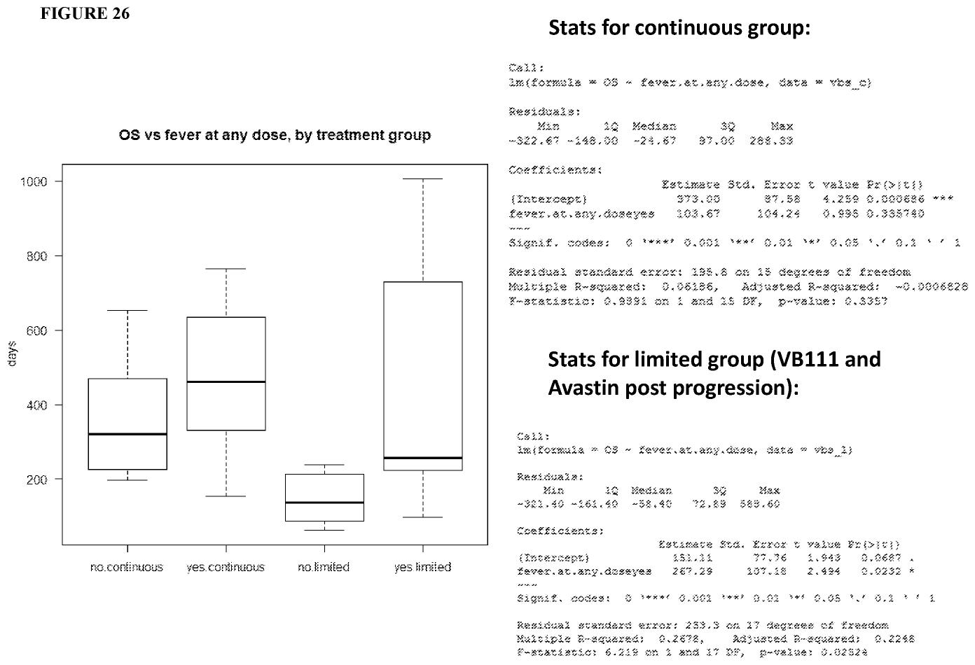

Second, the correlations between OS and subjects exhibiting a fever at any dose of vector were assessed by treatment group: Continuous treatment group or Limited treatment group. FIG. 26 shows that subjects exhibiting a fever after any dose of vector in both the Continuous and Limited treatment groups had an increased OS compared with subjects who did not exhibit a fever after any dose of vector.

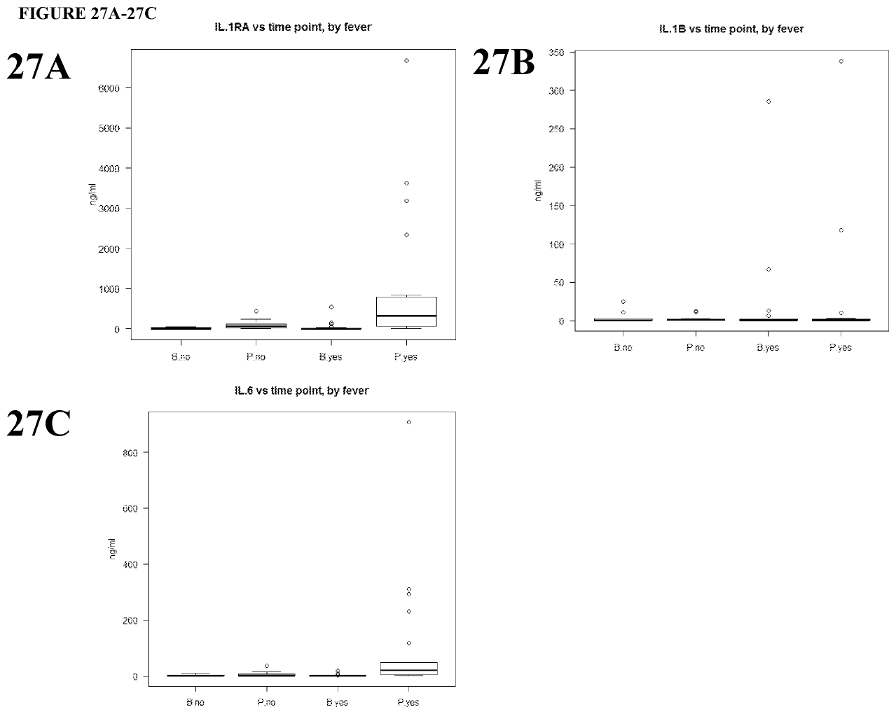

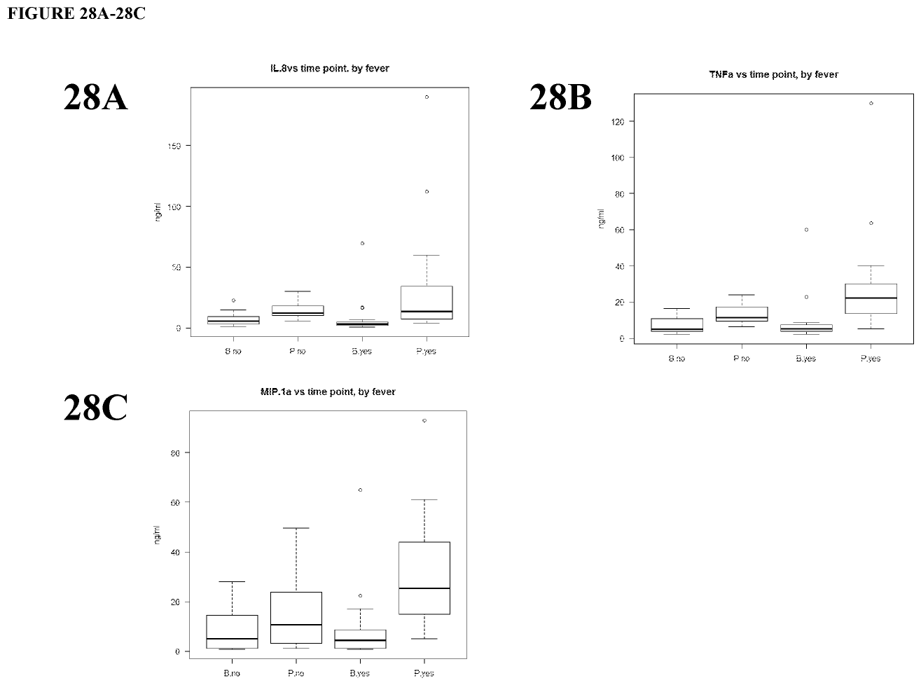

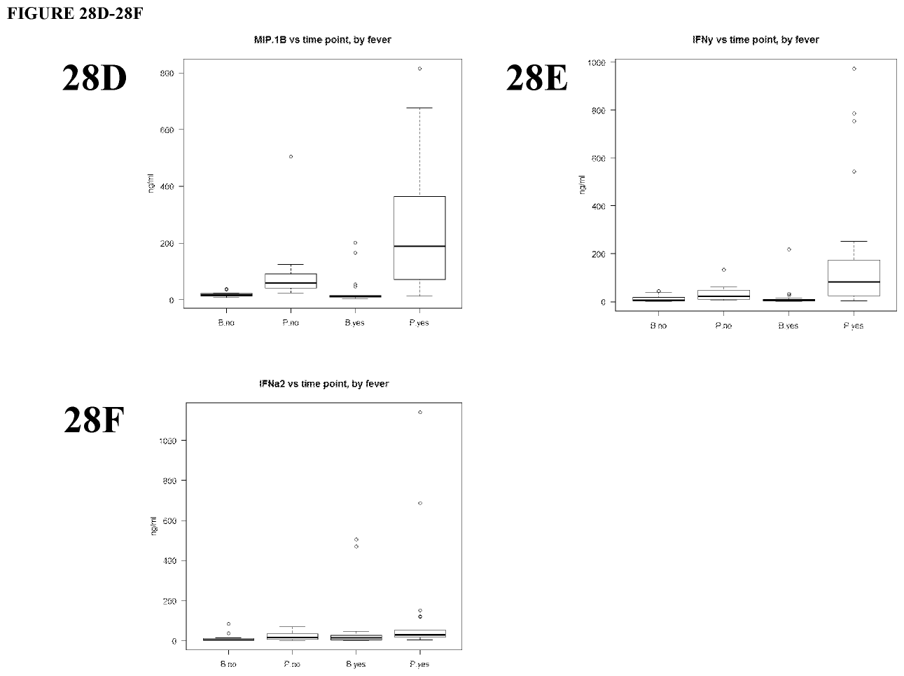

Third, the correlation between cytokines associated with fever and OS was assessed. Nine cytokines were analyzed: IL-1α, IL-1β, IL-6, IL-8, TNFα, MIP-1α, MIP-1β, IFN-α, and IFN-γ. FIG. 27A-27C show results for IL-1α, IL-1β, and IL-6 (respectively). Differences in IL-1α levels were significant with respect to both time and development of fever (FIG. 27A). Differences in IL-6 levels were significant with respect to time only (FIG. 27C). FIG. 28A-28F show the results for IL-8, TNFα, MIP-1α, MIP-1β, IFN-γ, and IFN-α (respectively). Differences in IL-8 levels were significant with respect to time only (FIG. 28A). Differences in TNFα levels were significant with respect to time and development of fever (FIG. 28B). Differences in MIP-1α levels were significant with respect to time and development of fever (FIG. 28C). Differences in MIP-1β levels were significant with respect to time and development of fever (FIG. 28D). Differences in IFN-γ levels were significant with respect to time and development of fever (FIG. 28E).

### Example 8

Objective. Treatment with Ad5-PPE-1-3X-Fas-c in combination with weekly Paclitaxel has shown in a phase 2 trial to prolong overall survival in patients with recurrent platinum resistant ovarian cancer. A febrile post-dosing response was associated with improved survival supporting the role of the immune system as part of VB-111's mechanism of action. In this example, patient tumor biopsy data was assessed to further characterize the intratumoral immunologic activity of Ad5-PPE-1-3X-Fas-c.

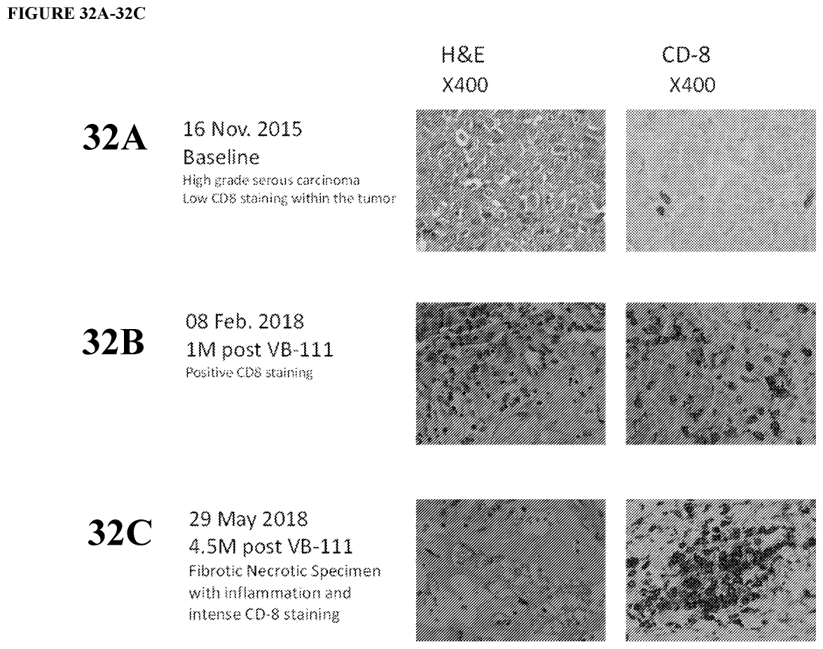

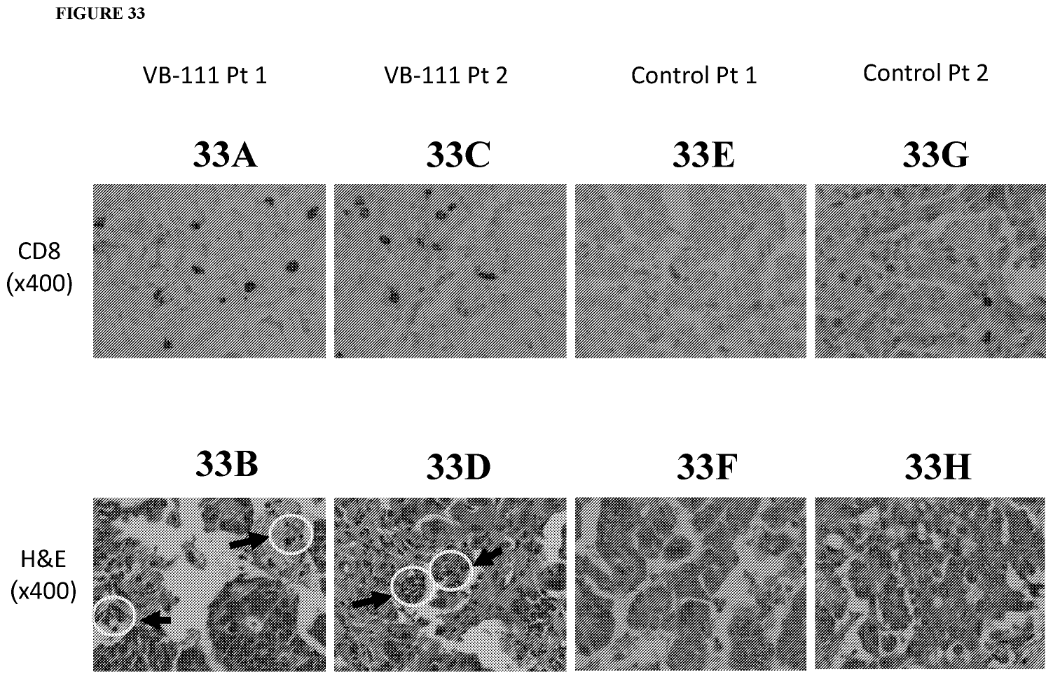

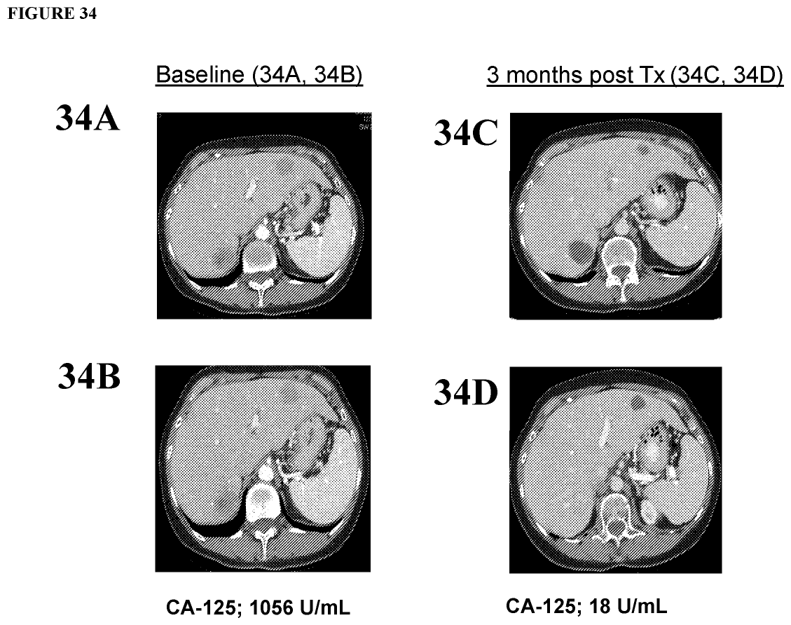

Method: Post treatment biopsies were obtained from 3 patients with recurrent platinum-resistant ovarian cancer treated with intravenous 1×10¹³ VPs of Ad5-PPE-1-3X-Fas-c every 2 months in combination with weekly paclitaxel. One patient was in study NCT03398655 (FIG. 32) and two patients were in study NCT01711970 (FIG. 33). Results were compared to pre-treatment specimens and to 12 untreated controls. H&E and Immunohistochemistry (IHC) was performed for CD8⁺ and CD4⁺ intratumoral T cells.

Results: Specimens taken before treatment with Ad5-PPE-1-3X-Fas-c showed no or minimal T cell infiltration in the tumor (FIG. 32A and FIG. 33E-33H). One month after Ad5-PPE-1-3X-Fas-c treatment metastatic lesions demonstrated tumor infiltrated with CD8⁺ T Cells (up to 74 CD8⁺ cells/HPF) and CD4⁺ T cells (FIG. 32B). Immunohistochemistry staining shows regions of apoptotic tumor cells (FIG. 33B, 33D (red circles)) and increased tumor infiltrating CD8 lymphocytes in Ad5-PPE-1-3X-Fas-c treated patients (FIG. 33A, 33C) compared to controls (FIG. 33E-33H).

At 4.5 months following first drug administration (post 3^{rd} dose) a liver lesion showed necrotic and fibrotic tissue with no viable tumor, lymphocytic aggregate, intensive staining for CD8 (157 CD8⁺ cells/HPF), intense staining for CD4 and pigmented macrophages (FIG. 32C).

CA-125 levels were also measured before treatment (baseline) and after three months of treatment with Ad5-PPE-1-3X-Fas-c combined with paclitaxel. In this example, CA-125 levels at baseline were 1056 U/ml (FIG. 34A, 34B). Three months following treatment with Ad5-PPE-1-3X-Fas-c every eight weeks and paclitaxel every week, the CA-125 levels were 18 U/mL (FIG. 34C, 34D).

Conclusion: Pathologic findings following Ad5-PPE-1-3X-Fas-c treatment suggest the induction of an Immunotherapeutic effect manifested as tumor infiltration with CD8⁺ T cells, and evidence of tumor necrosis. The presence of tumor-infiltrating lymphocytes is an important prognostic factor in ovarian cancer, and may contribute to the favorable survival outcome seen in the Ad5-PPE-1-3X-Fas-c phase II study. A pivotal phase III study evaluating the efficacy and safety of Ad5-PPE-1-3X-Fas-c combination with weekly Paclitaxel compared to Paclitaxel and Placebo is currently ongoing.

1.-15. (canceled)

16. A method for treating a tumor comprising administering a vector comprising a Fas-chimera gene operably linked to an endothelial cell-specific promoter to a subject who is capable of exhibiting a change in at least one plasma biomarker or cell surface biomarker after administration of at least one priming dose of the vector, wherein the subject is to be administered a therapeutically effective dose of the vector after the subject exhibits a change in at least one plasma biomarker or cell surface biomarker after the administration of a priming dose of the vector.

17. The method of claim 16, wherein the at least one plasma biomarker or cell surface biomarker is selected from the group consisting MCP-1, MIP-1, MIP-2, MIP-1α, MIP-1β, MIG, RANTES, IP-10, IL-1α, IL-1β, IL-2, IL-3, IL-4, IL-5, IL-6, IL-8, IL-9, IL-10, IL-12, IL-13, IL-15, IL-17α, IL-22, IL-23, IL-35, LIF, TNF-α, TNF-β, TGF-β1, VEGF, G-CSF, GM-CSF, IFN-α, IFN-β, IFN-γ, M-CSF, IL-1Ra, eotaxin, CA-125, and a combination thereof.

18. The method of claim 16, wherein the change in plasma biomarker or cell surface marker is an increase in the level of at least one plasma biomarker or cell surface marker.

19. The method of claim 18, wherein the plasma biomarker or cell surface marker that exhibits an increase in the level after the administration of the priming dose comprises at least one of the markers selected from the group consisting of IL-6, MCP-1, MIP-2, MIG, RANTES, MIP-1α, MIP-1β, IP-10, IL-2, IL-10, LIF, TNF-α, TGF-β1, VEGF, IL-17α, G-CSF, GM-CSF, IFN-γ, IL-1α, IL-1β, IL-12, IL-13, IL-15, IL-9, IL-22, IL-23, IL-35, M-CSF, IL-1Ra, and a combination thereof

20. The method of claim 16, wherein the change in plasma biomarker or cell surface marker is a decrease in the level at least one plasma biomarker or cell surface marker.

21. The method of claim 20, wherein the plasma biomarker or cell surface marker that exhibits a decrease in the level comprises at least one of the markers selected from IL-10, IL-4, IL-3, IL-5, IL-13, IL-2, LIF, TNF-α, CA-125, and a combination thereof.

22. The method of claim 16, wherein the vector consists of the nucleotide sequence set forth in SEQ ID NO: 19.

23. The method of claim 16, wherein the vector is an isolated virus having European Collection of Cell Cultures (ECACC) Accession Number 13021201.

24. The method of claim 16, wherein the subject is to be further administered an effective amount of one or more chemotherapeutic agents.

25. The method of claim 24, wherein the one or more chemotherapeutic agents are selected from the group consisting of altretamine, raltritrexed, topotecan, paclitaxel, docetaxel, cisplatin, carboplatin, oxaliplatin, liposomal doxorubicin, gemcitabine, cyclophosphamide, vinorelbine, ifosfamide, etoposide, altretamine, capecitabine, irinotecan, melphalan, pemetrexed, bevacizumab, and albumin bound paclitaxel.

26. A method of identifying a responder to a Fas-chimera gene therapy, comprising:
(a) administering at least one priming dose of a vector comprising a Fas-chimera gene operably linked to an endothelial cell-specific promoter to a subject in need thereof;
(b) wherein the subject has a change in at least one plasma biomarker or cell surface biomarker as a result of the administration of the priming dose in (a); and
(c) the subject having a change in at least one plasma biomarker or cell surface biomarker in (b) is to be administered a therapeutically effective dose of the vector.

27. The method of claim 26, wherein the at least one plasma biomarker or cell surface biomarker is selected from the group consisting MCP-1, MIP-1, MIP-2, MIP-1α, MIP-1β, MIG, RANTES, IP-10, IL-1α, IL-1β, IL-2, IL-3, IL-4, IL-5, IL-6, IL-8, IL-9, IL-10, IL-12, IL-13, IL-15, IL-17α, IL-22, IL-23, IL-35, LIF, TNF-α, TNF-β, TGF-β1, VEGF, G-CSF, GM-CSF, IFN-α, IFN-β, IFN-γ, M-CSF, IL-1Ra, eotaxin, CA-125, and a combination thereof.

28. The method of claim 26, wherein the change in plasma biomarker or cell surface marker is an increase in the level of at least one plasma biomarker or cell surface marker.

29. The method of claim 28, wherein the plasma biomarker or cell surface marker that exhibits an increase in the level after the administration of the priming dose comprises at least one of the markers selected from the group consisting of IL-6, MCP-1, MIP-2, MIG, RANTES, MIP-1a, MIP-1β, IP-10, IL-2, IL-10, LIF, TNF-α, TGF-β1, VEGF, IL-17α, G-CSF, GM-CSF, IFN-γ, IL-1α, IL-1β, IL-12, IL-13, IL-15, IL-9, IL-22, IL-23, IL-35, M-CSF, IL-1Ra, and a combination thereof

30. The method of claim 26, wherein the change in plasma biomarker or cell surface marker is a decrease in the level at least one plasma biomarker or cell surface marker.

31. The method of claim 30, wherein the plasma biomarker or cell surface marker that exhibits a decrease in the level comprises at least one of the markers selected from IL-10, IL-4, IL-3, IL-5, IL-13, IL-2, LIF, TNF-α, CA-125, and a combination thereof.

32. The method of claim 26, wherein the vector consists of the nucleotide sequence set forth in SEQ ID NO: 19.

33. The method of claim 26, wherein the vector is an isolated virus having European Collection of Cell Cultures (ECACC) Accession Number 13021201.

34. The method of claim 26, wherein the subject is to be further administered an effective amount of one or more chemotherapeutic agents.

35. The method of claim 34, wherein the one or more chemotherapeutic agents are selected from the group consisting of altretamine, raltritrexed, topotecan, paclitaxel, docetaxel, cisplatin, carboplatin, oxaliplatin, liposomal doxorubicin, gemcitabine, cyclophosphamide, vinorelbine, ifosfamide, etoposide, altretamine, capecitabine, irinotecan, melphalan, pemetrexed, bevacizumab, and albumin bound paclitaxel.
